(19) United States
(12) Patent Application Publication
(10) Pub. No.: US 2005/0164945 A1
(43) Pub. Date: Jul. 28, 2005

(54) Endotheliase-1 ligands

(76) Inventors: Andrew Nixon, Hanover, MA (US); Edwin L. Madison, San Francisco, CA (US)

Correspondence Address: FISH & RICHARDSON PC, 225 FRANKLIN ST, BOSTON, MA, 02110, US

(21) Appl. No.: 10/969,719

(22) Filed: Oct. 20, 2004

Related U.S. Application Data
(60) Provisional application No. 60/513,173, filed on Oct. 21, 2003.

(51) Int. Cl. A61K 38/08; A61K 38/10

(52) U.S. Cl. 514/14; 514/15; 514/16; 514/17; 530/326; 530/327; 530/328; 530/329; 530/330

(57) ABSTRACT

The disclosure describes compounds that can include a peptide or Kunitz domain that binds endotheliase 1 (ET1). The compounds can be used, e.g., to reduce angiogenesis in a subject having or at risk for a neoplastic disorder, modulate the activity of an ET1-expressing cell, modulate proteolysis of a biological structure, detect endotheliase activity or protein in a sample, and detect ET1 protein in a subjects.

### CROSS-REFERENCE TO RELATED APPLICATIONS

This application claims priority to U.S. application Ser. No. 60/513,173, filed on Oct. 21, 2003, the contents of which are incorporated by reference.

### BACKGROUND

Angiogenesis is the biological process of producing new blood vessels by sprouting a new branch from an existing blood vessel. While angiogenesis is essential for normal development and growth, it rarely occurs in adulthood except under strictly regulated circumstances (e.g., wound healing; see, for example, Moses et al., Science, 248:1408-1410, 1990). Angiogenesis also occurs in a number of diseases, such as cancer, in which new vessels are formed to support the growth and proliferation of tumors or other unwanted tissue.

Blood vessels are composed of endothelial cells surrounded by a basement membrane. One of the first steps in angiogenesis is the degradation of the basement membrane by proteolytic enzymes produced by endothelial cells to form a breach in the membrane through which endothelial cells can migrate and proliferate to form a new vessel sprout. This step can be initiated as follows. First, components of the plasminogen activator (PA)-plasmin system stimulate a protease cascade that results in high concentrations of plasmin and active matrix metalloproteinases (MMPs) at the site of angiogenesis. This increased proteolytic activity leads to degradation of the extracellular matrix (ECM) and invasion of the vessel basal lamina. The release of ECM degradation products stimulates activity of local growth factors and chemotaxis of endothelial cells.

Numerous pathological conditions are associated with unwanted angiogenesis. For example, tumors can induce angiogenesis in order to grow beyond minimal size and to metastasize (Hanahan D. and Folkman J. (1996) Cell 86:353-64). Tumor development is associated with increased release of angiogenesis factors, most prominently of vascular endothelial growth factor (VEGF) (Brown L. F., et al. (1997) Exs. 79:233-69). Other disorders characterized by unwanted angiogenesis include, for example, tissue inflammation, arthritis, diabetic retinopathy, and macular degeneration by neovascularization of retina (see, e.g., Folkman et al. (1987) Science 235:442-447).

The endotheliases are a class of membrane proteases that are expressed on cells, particularly endothelial cells, and that may participate in angiogenesis.

### SUMMARY

In one aspect, the invention features a compound (e.g., an isolated compound) that includes a peptide that binds endotheliase 1 (ET1, e.g., human ET1), e.g., with a K_{d} of less than 50 μM. In one embodiment, the peptide independently binds ET1. In one embodiment, the peptide is composed of less than 30, 28, 22, 20, 18, 16, 14, 12, 10, or 8 amino acids. For example, the peptide is composed of between 6-12, 8-14, 10-16, 10-20, 12-18, 14-20, or 16-28 amino acids. The peptide can include two cysteines that form a disulfide bond. For example, between four and twelve, or five, six, seven, or eight amino acids separate the two cysteines. In another embodiment, the cysteine is replaced by another intra-molecular covalent linkage, e.g., as described herein.

The ET1-binding compound may bind to human ET1 with high affinity and specificity, e.g., the compound specifically binds to ET1. As used herein, “specific binding” refers to the property of the compound: (1) to bind to ET1, e.g., human ET1, with an affinity (K_{d}) of less than 50 μM, and (2) to preferentially bind to ET1, e.g., human ET1, with an affinity that is at least two-fold better than its affinity for a non-specific antigen. For example, the ET1-binding compound can preferentially bind to ET1 at least 10-fold, 50-fold, 100-fold, or better (smaller K_{d}) than its affinity for binding to a non-specific antigen (e.g., BSA, casein) other than ET1. The compound can have a K_{d} of less than 50 μM, 1 μM, 500 nM, 200 nM, 100 nM, 50 nM, 5 nM, 500 pM, or 10 pM, e.g., between 500 nM and 500 pM, or 200 nM and 1 nM.

In one embodiment, the compound binds to ET1 and modulates the proteolytic activity of ET1. In one embodiment, the compound inhibits ET1. For example, the compound can have a Kᵢ of less than 50 μM, 1 μM, 500 nM, 200 nM, 100 nM, 50 nM, 5 nM, 500 pM, or 10 pM, e.g., between 500 nM and 500 pM, or 200 nM and 1 nM.

In one embodiment, the compound specifically inhibits ET1, e.g., relative to another protease (e.g., a protease whose protease domain is between 30-90% identical to the ET1 protease domain, or between 30-60% identical to the ET1 protease domain). For example, the compound does not inhibit other proteases, e.g., non-ET1 proteases such as trypsinogen-IV, membrane-type serine proteases-1, -6, -7, urokinase-like plasminogen activator (uPA), trypsin, factor IIa, plasmin (Plm), and/or factor Xa or ET2 (endotheliase-2), e.g., the compound inhibits such other proteases with an inhibition constant at least 2-, 5-, or 10-fold worse (e.g., numerically greater) than the inhibition constant for ET1 (i.e., the compound does not inhibit the other proteases as well as it inhibits ET1). In one embodiment, the compound inhibits both ET1 and ET2. The compound may be specific for these two endotheliases, but not other endotheliases.

In one embodiment, the peptide binds the active site of ET1. For example, the peptide adopts a conformation that is compatible with the van der Waals surface of the ET1 active site. For example, at least one amino acid in the peptide is within 10, 7, 5, or 3 Angstroms of the active site serine of ET1 or the active site histidine of ET1, when the compound is bound to ET1.

In an embodiment, the compound inhibits the activity of ET1 with an IC₅₀ of less than 50 μM, 1 μM, 500 nM, 200 nM, 100 nM, 50 nM, 5 nM, 500 pM, or 10 pM, e.g., between 500 nM and 500 pM, or 200 nM and 1 nM. In an embodiment, the peptide is not cleaved by ET1, e.g., after a 12 hour incubation with 100 nM rET1. In an embodiment, the peptide binds to an ET1 molecule at least 5, 10, 50, 100, or 1000-fold more tightly than the peptide binds to an ET1 molecule that has been reacted with 4-(2-aminoethyl)benzene sulfonyl fluoride (AEBSF). In one embodiment, the peptide does not bind and/or inhibit a non-ET1 protease such as trypsinogen-IV, membrane-type serine proteases-1, -6, -7, urokinase-like plasminogen activator (uPA), trypsin, factor IIa, plasmin (Plm), and/or factor Xa or ET2.

In one embodiment, the compound, as an isolated preparation, is greater than 85%, 90%, 95%, or 99% pure.

In one embodiment, the compound includes a protein that contains the peptide, and the protein is greater than 32 amino acids in length, e.g., at least 80 or 200 amino acids in length. In another embodiment, the compound includes a protein that is less than 30, 28, 22, 20, 18, 16, 14, 12, 10, or 8 amino acids in length and that includes the peptide.

In one embodiment, the peptide is non-naturally occurring, e.g., not present as a peptide encoded in the human genome, or not present as a subsequence in an amino acid sequence encoded in the human genome. For example, the peptide is not a naturally occurring substrate of ET1.

The compound can have one or more of the following properties when administered to a tissue or organism: inhibit angiogenesis, accumulates at sites of angiogenesis in vivo, and inhibit proteolysis of vessel basement membrane (e.g., showing a statistically significant change in vessel basement membrane proteolysis in vitro or in vivo). Exemplary assays are described below in “Assays for ET1 binding ligands.” The compound can produce a statistically significant effect in one or more of such assays. In one embodiment, the compound has a statistically significant effect (e.g., on an angiogenic process) in one or more of the following assays: a cornea neovascularization assay; a chick embryo chorioallantoic membrane model assay; an assay using SCID mice injected with tumors (e.g., tumors arising from injection of DU145 or LnCaP cell lines, as described in Jankun et al. (1997) Canc. Res. 57: 559-563); or an assay in which mice are injected with bFGF, to stimulate angiogenesis (e.g., as described by Min et al. (1996) Canc. Res. 56: 2428-2433). Exemplary effects in these assays include an at least 1.5, 2, 5, 10, or 20-fold difference relative to a negative control (e.g., no compound).

Typically, the compound consists of a single polypeptide chain that includes the peptide. In one embodiment, the compound (e.g., the polypeptide) is not glycosylated.

In one embodiment, the compound includes or is physically attached to a moiety that prolongs serum residence time. For example, the moiety can be attached to a terminus of the protein (e.g., the amino or carboxy terminus). An embodiment of this example is a fusion of the peptide to a serum albumin or to an immunoglobulin domain, e.g., to an immunoglobulin constant domain, e.g., to an Fc domain. In one embodiment, the moiety is a hydrophilic water-soluble polymer (e.g., a polyethylene glycol or other polymer described herein), e.g., having a molecular weight of at least 5 kDa, 8 kDa, 10 kDa, 15 kDa, 20 kDa, or 30 kDa (e.g., between 10 and 40 kDa). The compound may also include a plurality of peptides, at least one of which is the peptide that binds to ET1. For example, the compound may include a plurality of ET1-binding peptides (e.g., at least 2, 3, 4, or 5 peptides), e.g., the peptide can be multimerized (e.g., in at least 2, 3, 4, or 5 copies).

In one embodiment, the peptide includes the following amino acid sequence:

| | |
|---|---|
| X₁-X₂-X₃-C₄-X₅-X₆-X₇-X₈-X₉-X₁₀- | (SEQ ID NO:212) |
| C₁₁-X₁₂-X₁₃-X₁₄, | |

wherein X is any amino acid (e.g., except cysteine) wherein X₁, X₂, and X₃ can be absent, one or both of X₅ and X₆ can be absent, and X₁₂, X₁₃ and X₁₄ can be absent.

For example, the peptide includes the following sequence:

C₄-X₅-X₆-X₇-X₈-X₉-X₁₀-C₁₁, or

wherein X₅ is aliphatic,
X₆ is hydrophilic,
X₇ is hydrophilic,
X₈ is hydrophilic (e.g., acidic),
X₉ is aliphatic, P, S, or T, and
X₁₀ is F, Y. P, or H, or a sequence that differs by two or fewer amino acid substitutions, insertions, or deletions from the above sequence.

In one embodiment, X₅ is L or I, X₆ is S or T, X₇ is R or K, X₈ is D, X₉ is I, L, P, or T, and X₁₀ is P. In one embodiment, the peptide includes one or more of the following features: X₁ is R, X2 is R or V, and, X3 is K, Y, R, or F.

In one embodiment,
X₁₂ is an amino acid with three or fewer side chain carbons (e.g., T, S, or V),
X₁₃ is any amino acid (e.g., V, I, N, T, or H), and
X₁₄ is hydrophilic (e.g., K, H, F, R, or Y).

In one embodiment, the peptide includes the following sequence:

| | |
|---|---|
| X₁-X₂-X₃-C-X₅-S-R-D-L-P-C-X₁₂-X₁₃- | (SEQ ID NO:210) |
| X₁₄ | |
| or | |

a sequence that differs by two or fewer amino acid substitutions, insertions, or deletions from the above sequence,
wherein X₁, X₂, X₃, X₁₂, X₁₃, and X₁₄ are any amino acid or absent, and X₅ is L or I.

In one embodiment, the peptide includes the following sequence:

| | |
|---|---|
| X₁-X₂-X₃-C-X₅-S-R-D-L-P-C-X₁₂-X₁₃- | (SEQ ID NO:210) |
| X₁₄ | |
| or | |

wherein X₂, X₃, X₁₃, and X₁₄ are any amino acid or absent, X₁ is R, M, or K (e.g., R or K), X₅ is L or I, and X₁₂ is S, V, or T (e.g., S or T), or a sequence that differs by two or fewer amino acid substitutions, insertions, or deletions from the above sequence. For example, X₂ is R.

In one embodiment, the peptide includes the following sequence:

| | |
|---|---|
| C₄-X₅-X₆-X₇-X₈-X₉-X₁₀-C₁₁ | (SEQ ID NO:214) |

For example, X₅ is K or R,
X₆ is G,
X₇ is Y or F,
X₈ is Y, W, or A,
X₉ is P, and
X₁₀ is D, or the peptide includes a sequence that differs by two or fewer amino acid substitutions, insertions, or deletions from the above sequence. For example, the peptide includes C—K-G-X₇—P-D-C (SEQ ID NO:211), wherein X₇ is Y or F, or a sequence that differs by one amino acid substitution, insertion, or deletion.

In another example, X₅ is K or R,
X₆ is G,
X₇ is any amino acid,
X₈ is any amino acid,
X₉ is P,
X₁₀ is D or E.

The peptide can include the following sequence:

| | |
|---|---|
| X₁-X₂-X₃-C₄-X₅-X₆-X₇-X₈-X₉-X₁₀- | (SEQ ID NO:215) |
| C₁₁, | |

wherein X₂ is W, F, or Y,
X₅ is K or R,
X₆ is C
X₇ is Y or F,
X₈ is Y, W, or A,
X₉ is P, and
X₁₀ is D, or a sequence that differs by two or fewer amino acid substitutions, insertions, or deletions from the above sequence.

For example, the peptide can include one or more of the following features: X₃ is R, P, K, or V; X₁ is R, S, T, or G; X₁ is G; and X₅ is K.

The peptide can include:

| | |
|---|---|
| C₄-X₅-X₆-X₇-X₈-X₉-X₁₀-C₁₁-X₁₂-X₁₃- | (SEQ ID NO:216) |
| X₁₄, | |

or a sequence that differs by two or fewer amino acid substitutions, insertions, or deletions from the above sequence, wherein X₅ is K or R,
X₆ is G,
X₇ is Y or F,
X₈ is Y, W, or A,
X₉ is P, and
X₁₀ is D,
X₁₂ is V, I, or E,
X₁₃ is W, and
X₁₄ is Q.

The peptide can include:

| | |
|---|---|
| C₄-X₅-X₆-X₇-X₈-X₉-X₁₀-C₁₁-X₁₂-X₁₃- | (SEQ ID NO:217) |
| X₁₄-X₁₅-X₁₆, | |

wherein X₅ is K or R,
X₆is G,
X₇ is Y or F,
X₈ is Y, W, or A,
X₉ is P, and
X₁₀ is D,
X₁₂ is V, I, or E,
X₁₃ is W, and
X₁₄ is Q,
X₁₅ is T, F, or I, and
X₁₆ is W, F, or A.

In one embodiment, X₅ is K or R,
X₆ is G,
X₇ is any amino acid,
X₈ is any amino acid,
X₉ is P,
X₁₀ is D or E.

For example, one or both of X₇ and X₈ is aromatic. Further, X₂ can be W.

In one embodiment, the peptide includes, between C₅ and C₁₁, an amino acid sequence selected from the group consisting of: KGFAPD (SEQ ID NO:55), KGFWPD (SEQ ID NO:56), KGLYPD (SEQ ID NO:57), KGLVPE (SEQ ID NO:58), KGYAPD (SEQ ID NO:59), KGYYPD (SEQ ID NO:60), KGYWPD (SEQ ID NO:61), KGYFPD (SEQ ID NO:62), KGYEPD (SEQ ID NO:63), KDYPPD (SEQ ID NO:64), KGLYPD (SEQ ID NO:65), RGFYPD (SEQ ID NO:66), RGFWPD (SEQ ID NO:67), and RGYAPD (SEQ ID NO:68),
or an amino acid sequence selected from an amino acid sequence that differs by no more than one amino acid substitution, insertion or deletion from a sequence in the above group.

In one embodiment, the cysteines are separated by an amino acid sequence that comprises X₁—R-D-X₂—P, wherein X₁ is S or T. For example, X₂ is L, T, or I.

The peptide can include the amino acid sequence:
C—X₃—X₁—R-D-X₂—P—C (SEQ ID NO:209), wherein X3 is any amino acid.

In one embodiment, the cysteines are separated by an amino acid sequence selected from the group consisting of: LSRDTP (SEQ ID NO:69), LSRDLP (SEQ ID NO:70), ESRDLP (SEQ ID NO:71), ESRDIP (SEQ ID NO:72), and TRDLP (SEQ ID NO:73).

The invention also provides a nucleic acid that includes a sequence encoding a peptide or protein described herein.

The compound can further include a detectable label, a toxin, e.g., a cytotoxin, and/or a carrier molecule. In one embodiment, the in vivo half life of the compound including the carrier molecule is at least two, five, or twenty times greater than the in vivo half life of an otherwise identical compound that does not include the carrier molecule. In one embodiment, the carrier molecule is a hydrophilic polymer, e.g., PEG. In one embodiment, the carrier molecule is a serum albumin. For example, the serum albumin and the peptide are components of the same polypeptide chain.

In one embodiment, the compound is produced in a cell. In another embodiment, the compound is produced by synthetic chemistry.

In another aspect, the invention features a compound (e.g., an isolated compound) comprising a peptide that binds endotheliase 1 (ET1) with a K_{d} of less than 50 μM. For example, the peptide can have a K_{d} of less than 50 μM, 1 μM, 500 nM, 200 nM, 100 nM, 50 nM, 5 nM, 500 pM, or 10 pM, e.g., between 500 nM and 500 pM, or 200 nM and 1 nM. The peptide can include two cysteines that form a disulfide bond and an amino acid that differs by fewer than three amino acid substitutions, insertions, or deletions from an amino acid sequence selected from the group consisting of: RRKCISRDIPCVTH, RRYCISRDIPCVTH, RVRCISRDIPCVTH, RRFCISRDIPCVTH, RVKCISRDIPCVTH, KMRCISRDIPCTVK, KMRCLSRDIPCSIH, KMRCLSRDIPCVNF, KMRCISRDIPCTVF, KMRCISRDIPCTTR, KMRCISRDIPCSHY, RYPCKGFYPDCGYP, GWRCKGYYPDCGYP, SWRCKGYYPDCGYP, TWVCKGYYPDCGYP, GWRCKGYYPDCGYP, GWKCKGYYPDCGYP, GWRCKGYYPDCGYP, KHICRGFYPDCVWQ, KHICRGYYPDCVWQ, KHICRGYYPDCIWQ, KHICRGFYPDCVWQ, and KHICRGYYPDCEWQ (SEQ ID NOs 9-31 respectively). The peptide can include, e.g., an amino acid that differs by fewer than three amino acid substitutions, insertions, or deletions from an amino acid sequence selected from the group consisting of: QMRRKCISRDIPCVTH, QVRRYCISRDIPCVTH, RSRVRCISRDIPCVTH, SGRRFCISRDIPCVTH, MARVKCISRDIPCVTH, AGKMRCISRDIPCTVK, AGKMRCLSRDIPCSIH, AGKMRCLSRDIPCVNF, AGKMRCISRDIPCTVF, AGKMRCISRDIPCTTR, AGKMRCISRDIPCSHY, GWRYPCKGFYPDCGYP, NTGWRCKGYYPDCGYP, RASWRCKGYYPDCGYP, RETWVCKGYYPDCGYP, RAGWRCKGYYPDCGYP, QLGWKCKGYYPDCGYP, SSGWRCKGYYPDCGYP, AGKHICRGFYPDCVWQ, AGKHICRGYYPDCVWQ, AGKHICRGYYPDCIWQ, AGKHICRGFYPDCVWQ, and AGKHICRGYYPDCEWQ (SEQ ID NOs 32-54 respectively).

Kunitz Domains

In another aspect, the invention features an isolated protein that includes a Kunitz domain that binds endotheliase 1 (ET1), e.g., with a K_{d} of less than 50 μM. The compound can have a K_{d} of less than 50 μM, 1 μM, 500 nM, 200 nM, 100 nM, 50 nM, 5 nM, 500 pM, or 10 pM, e.g., between 500 nM and 500 pM, or 200 nM and 1 nM. In one embodiment, the Kunitz domain independently binds to ET1. For example, the Kunitz domain includes the amino acid sequence:

X₁—X₂—X₃—X₄—C₅—X₆—X₇—X₈—X₉—X₉ₐ—X₁₀—X₁₁—X₁₂—X₁₃—C₁₄—X₁₅—X₁₆—X₁₇—X₁₈—X₁₉—X₂₀—X₂₁—X₂₂—X₂₃—X₂₄—X₂₅—X₂₆—X₂₇—X₂₈—X₂₉—X₂₉ₐ—X_{29b—X}_{29c}—C₃₀—X₃₁—X₃₂—X₃₃—X₃₄—X₃₅—X₃₆—X₃₇—C₃₈—X₃₉—X₄₀—X₄₁—X₄₂—X₄₂ₐ—X_{42b}—X₄₃—X₄₄—X₄₅—X₄₆—X₄₇—X₄₈—X₄₉—X₅₀—C₅₁—X₅₂—X₅₃—X₅₄—C₅₅—X₅₆—X₅₇—X₅₈ (SEQ ID NO:5), wherein X is any amino acid other than cysteine. In one embodiment, one or more Of X₉ₐ, X₄₂ₐ, and X_{42b} are absent. The domain can have one or more of the following properties: X₁₅ is basic, e.g., R, X₁₁ is P or hydrophilic, e.g., R, K, Q, E, T,. X₁₃ is aromatic, or hydrophilic, e.g., P, F, E, T, or Q, X₁₇ is aliphatic, e.g., A, I, L, or M, or hydrophilic, e.g., D, Y, or S, X₁₈ is any amino acid, X₁₉ is hydrophilic, e.g., T, K, D, R, H, N, Q, or aliphatic, e.g., I, V, or P, and X₃₄ is any amino acid (e.g., hydrophobic, aliphatic, or aromatic). In one embodiment, X₃₉ is any amino acid except Cys and X₄₀ is Ala or Gly.

Other exemplary properties include:
X₁₁ is R, X₁₃ is P, X₁₅ is R, X₁₇ is D, X₁₈ is F, X₁₉ is H, X₃₄ is H;
X₁₁ is K, X₁₃ is F, X₁₅ is R, X₁₇ is M, X₁₈ is D, X₁₉ is I, X₃₄ is I;
X₁₁ is Q, X₁₃ is P, X₁₅ is R, X₁₇ is A, X₁₈ is I, X₁₉ is S, X₃₄ is V;
X₁₁ is K, X₁₃ is E, X,₅ is R, X₁₇ is S, X₁₈ is V, X₁₉ is Q, X₃₄ is N;
X₁₁ is P, X₁₃ is P, X₁₅ is R, X₁₇ is M, X₁₈ is F, X₁₉ is N, X₃₄ is N;
X₁₁ is K, X₁₃ is T, X₁₅ is R, X₁₇ is D, X₁₈ is F, X₁₉ is P, X₃₄ is H;
X₁₁ is Q, X₁₃ is P, X₁₅ is R, X₁₇ is S, X₁₈ is V, X₁₉ is H, X₃₄ is F;
X₁₁ is P, X₁₃ is Q, X₁₅ is R, X₁₇ is Y, X₁₈ is Y, X₁₉ is R, X₃₄ is V;
X₁₁ is R, X₁₃ is P, X₁₅ is R, X₁₇ is Y, X₁₈ is F, X₁₉ is D, X₃₄ is I;
X₁₁ is T, X₁₃ is P, X₁₅ is R, X₁₇ is D, X₁₈ is I, X₁₉ is K, X₃₄ is R;
X₁₁ is P, X₁₃ is P, X₁₅ is R, X₁₇ is I, X₁₈ is M, X₁₉ is T, X₃₄ is R; and
X₁₁ is T, X₁₃ is T, X₁₅ is R, X₁₇ is A, X₁₈ is M, X₁₉ is V, X₃₄ is T.

In one embodiment, the Kunitz domain includes the amino acid sequence:

C₅-A-F—K-A-D-X₁₁-G-X₁₃—C₁₄—X₁₅-A-X₁₇—X₁₈—X₁₉—R—F—F—F—N—I—F-T-R-Q-C₃₀-E-E-F—X₃₄—Y-G-G-C₃₈—X₃₉—X₄₀—N-Q-N—R—F-E-S-L-E-E-C₅₁—K—K-M-C₅₅ (SEQ ID NO:7) or a sequence that differs by at least one, but no more than six, five, four, three, or two differences (e.g., a substitution, e.g., a conservative substitution, insertion, or deletion). In one embodiment, X₃₉ is any amino acid except Cys and X₄₀ is Ala or Gly.

Other exemplary properties include:
X₁₁ is R, X₁₃ is P, X₁₅ is R, X₁₇ is D, X₁₈ is F, X₁₉ is H, X₃₄ is H;
X₁₁ is K, X₁₃ is F, X₁₅ is R, X₁₇ is M, X₁₈ is D, X₁₉ is I, X₃₄ is I;
X₁₁ is Q, X₁₃ is P, X₁₅ is R, X₁₇ is A, X₁₈ is I, X₁₉ is S, X₃₄ is V;
X₁₁ is K, X₁₃ is E, X₁₅ is R, X₁₇ is S, X₁₈ is V, X₁₉ is Q, X₃₄ is N;
X₁₁ is P, X₁₃ is P, X₁₅ is R, X₁₇ is M, X₁₈ is F, X₁₉ is N, X₃₄ is N;
X₁₁ is K, X₁₃ is T, X₁₅ is R, X₁₇ is D, X₁₈ is F, X₁₉ is P, X₃₄ is H;
X₁₁ is Q, X₁₃ is P, X₁₅ is R, X₁₇ is S, X₁₈ is V, X₁₉ is H, X₃₄ is F;
X₁₁ is P, X₁₃ is Q, X₁₅ is R, X₁₇ is Y, X₁₈ is Y, X₁₉ is R, X₃₄ is V;
X₁₁ is R, X₁₃ is P, X₁₅ is R, X₁₇ is Y, X₁₈ is F, X₁₉ is D, X₃₄ is I;
X₁₁ is T, X₁₃ is P, X₁₅ is R, X₁₇ is D, X₁₈ is I, X₁₉ is K, X₃₄ is R;
X₁₁ is P, X₁₃ is P, X₁₅ is R, X₁₇ is I, X₁₈ is M, X₁₉ is T, X₃₄ is R; and
X₁₁ is T, X₁₃ is T, X₁₅ is R, X₁₇ is A, X₁₈ is M, X₁₉ is V, X₃₄ is T.

In another embodiment, the Kunitz domain includes the amino acid sequence:

M-H—S—F—C₅-A-F—K-A-D-X₁₁-G-X₁₃-C₁₄—X₁₅-A-X₁₇—X₁₈—X₉—R—F—F—F—N—I—F-T-R-Q-C₃₀-E-E-F—X₃₄—Y-G-G-C₃₈—X₃₉—X₄₀—N-Q-N—R—F-E-S-L-E-E-C₅₁—K—K-M-C₅₅-T-R-D-S-A-S—S-A-S-G-D-F-D- (SEQ ID NO:8). These exemplary Kunitz domains can have one or more of the following properties: X₁₅ is basic, e.g., R, X₁₁,is P or hydrophilic, e.g., R, K, Q, E, T, X₁₃ is aromatic, or hydrophilic, e.g., P, F, E, T, or Q, X₁₇ is aliphatic, e.g., A, I, L, or M, or hydrophilic, e.g., D, Y, or S, X₁₈ is any amino acid, X₁₉ is hydrophilic, e.g., T, K, D, R, H, N, Q, or aliphatic, e.g., I, V, or P, and X₃₄ is any amino acid (e.g., hydrophobic, aliphatic, or aromatic).

Other exemplary properties include:
X₁₁ is R, X₁₃ is P, X₁₅ is R, X₁₇ is D, X₁₈ is F, X₁₉ is H, X₃₄ is H;
X₁₁ is K, X₁₃ is F, X₁₅ is R, X₁₇ is M, X₁₈ is D, X₁₉ is I, X₃₄ is I;
X₁₁ is Q, X₁₃ is P, X₁₅ is R, X₁₇ is A, X₁₈ is I, X₁₉ is S, X₃₄ is V;
X₁₁ is K, X₁₃ is E, X₁₅ is R, X₁₇ is S, X₁₈ is V, X₁₉ is Q, X₃₄ is N;
X₁₁ is P, X₁₃ is P, X₁₅ is R, X₁₇ is M, X₁₈ is F, X₁₉ is N, X₃₄ is N;
X₁₁ is K, X₁₃ is T, X₁₅ is R, X₁₇ is D, X₁₈ is F, X₁₉ is P, X₃₄ is H;
X₁₁ is Q, X₁₃ is P, X₁₅ is R, X₁₇ is S, X₁₈ is V, X₁₉ is H, X₃₄ is F;
X₁₁ is P, X₁₃ is Q, X₁₅ is R, X₁₇ is Y, X₁₈ is Y, X₁₉ is R, X₃₄ is V;
X₁₁ is R, X₁₃ is P, X₁₅ is R, X₁₇ is Y, X₁₈ is F, X₁₉ is D, X₃₄ is I;
X₁₁ is T, X₁₃ is P, X₁₅ is R, X₁₇ is D, X₁₈ is I, X₁₉ is K, X₃₄ is R;
X₁₁ is P, X₁₃ is P, X₁₅ is R, X₁₇ is I, X₁₈ is M, X₁₉ is T, X₃₄ is R; and
X₁₁ is T, X₁₃ is T, X₁₅ is R, X₁₇ is A, X₁₈ is M, X₁₉ is V, X₃₄ is T.

In one embodiment, the protein differs by at least one, but no more than two, three, four, five, or six amino acids relative to an above protein. For example, the protein can differ at least one, but no more than two, three, four, five, or six amino acids that are located at least 5, 10, or 15 Angstroms from the protease contacting residues, e.g., as defined by a structural model listed herein. In one embodiment, the protein differs by fewer than three, two, one, or no amino acid differences (e.g., substitutions, e.g., conservative substitutions, insertions, or deletions) at positions 11, 15, 16, 17, 18, 19, 32, 34, 39, and 40 from a specific ET1-binding Kunitz domain sequence described herein. The protein can include, e.g., amino acids from a human Kunitz domain, e.g., at at least 80, 90, 95, or 100% of the remaining other positions.

The Kunitz domain of the protein can fold into a three dimensional structure that has an RMSD (Root Mean Square Deviation) of less than 4, 3, 2.5, 2.1, 2, or 1.8 Å² relative to a structural model listed herein.

In another aspect, the invention features a protein (e.g., an isolated protein) that includes a Kunitz domain that binds endotheliase 1 (ET1), e.g., with a K_{d} of less than 50 μM. The protein can have a K_{d} of better than (i.e., numerically less than) 50 μM, 1 pM, 500 nM, 200 nM, 100 nM, 50 nM, 5 nM, 500 pM, or 10 pM, e.g., between 500 nM and 500 pM, or 200 nM and 1 nM. The Kunitz domain can include an amino acid sequence that differs by no more than six, five, four, or three amino acid substitutions, insertions, or deletions from an amino acid sequence selected from the group:

| | |
|---|---|
| SEQ ID NO:74 | SFCAFKADRGPCRADFHRFFFNIFTRQCEEFHYGGCGGNQNRFESLEECKKMCTRDS |
| SEQ ID NO:75 | SFCAFKADKGFCRAMDIRFFFNIFTRQCEEFIYGGCGGNQNRFESLEECKKMCTRDS |
| SEQ ID NO:76 | SFCAFKADQGPCRAAISRFFFNIFTRQCEEFVYGGCEGNQNRFESLEECKKMCTRDS |
| SEQ ID NO:77 | SFCAFKADKGECRASVQRFFFNIFTRQCEEFNYGGCGGNQNRFESLEECKKMCTRDS |
| SEQ ID NO:78 | SFCAFKADPGPCRAMFNRFFFNIFTRQCEEFNYGGCSGNQNRFESLEECKKMCTRDS |
| SEQ ID NO:79 | SFCAFKADKGTCRGDFPRFFFNIFTRQCEEFHYGGCGGNQNRFESLEECKKMCTRDS |
| SEQ ID NO:80 | SFCAFKADQGPCRASVHRFFFNIFTRQCEEFFYGGCLGNQNRFESLEECKKMCTRDS |
| SEQ ID NO:81 | SFCAFKADPGQCRAYYRRFFFNIFTRQCEEFVYGGCMGNQNRFESLEECKKMCTRDS |
| SEQ ID NO:82 | SFCAFKADRGPCRAYFDRFFFNIFTRQCEEFIYGGCMGNQNRFESLEECKKMCTRDS |
| SEQ ID NO:83 | SFCAFKADTGPCRADIKRFFFNIFTRQCEEFRYGGCMGNQNRFESLEECKKMCTRDS |
| SEQ ID NO:84 | SFCAFKADPGPCRAIMTRFFFNIFTRQCEEFRYGGCLGNQNRFESLEECKKMCTRDS |
| SEQ ID NO:85 | SFCAFKADTGTCRAAMVRFFFNIFTRQCEEFTYGGCEGNQNRFESLEECKKMCTRDS |

In one embodiment, the Kunitz domain includes an amino acid sequence that differs by at least one, two, three, four, or five amino acid substitutions, insertions, or deletions from an aforementioned amino acid sequence. For example, the protein can differ at at least one, but no more than two, three, four, five, or six amino acids that are located at least 5, 10, or 15 Angstroms from the protease contacting residues, e.g., as defined by a structural model listed herein.

In one embodiment, the Kunitz domain differs from a naturally occurring human Kunitz domain by fewer than eight, seven, six, five, four, or three amino acids. For example, the Kunitz domain may be sufficiently human that when administered to a human, the domain does not cause an adverse immunogenic reaction. In one embodiment, positions other than 11, 15, 16, 17, 18, 19, 32, 34, 39, and 40 are identical to corresponding positions in a naturally occurring human Kunitz domain.

In one embodiment, the protein binds to ET1 with a K_{d} of less than 50 μM, 1 μM, 500 nM, 200 nM, 100 nM, 50 nM, 5 nM, 500 pM, or 10 pM, e.g., between 500 nM and 500 pM, or 200 nM and 1 nM.

In one embodiment, the protein binds to ET1 and modulates the proteolytic activity of ET1. In one embodiment, the protein inhibits ET1. For example, the protein can have a Kᵢ of better than (i.e., numerically less than) 50 μM, 1 μM, 500 nM, 200 nM, 100 nM, 50 nM, 5 nM, 500 pM, or 10 pM, e.g., between 500 nM and 500 pM, or 200 nM and 1 nM.

In one embodiment, the protein specifically inhibits ET1, e.g., relative to another protease (e.g., a protease whose protease domain is between 30-90% identical to the ET1 protease domain or between 30-60% identical to the ET1 protease domain). For example, the protein does not inhibit other proteases, e.g., non-ET1 proteases such as trypsinogen-IV, membrane-type serine proteases-1, -6, -7, urokinase-like plasminogen activator (uPA), trypsin, factor IIa, plasmin (Plm), and/or factor Xa or ET2, e.g., the protein inhibits such other proteases with an inhibition constant at least 2-, 5-, or 10-fold worse (e.g., numerically greater) than the inhibition constant for ET1 (i.e., the protein does not inhibit the other proteases as well as it inhibits ET1).

The Kunitz domain can include other features described herein.

In another aspect, the invention features an isolated protein that includes a Kunitz domain that binds endotheliase 1 (ET1) with a K_{d} of less than 50 μM. For example, the Kunitz domain independently binds to ET1. The Kunitz domain can include an amino acid sequence that differs by no more than four amino acid substitutions, insertions, or deletions from an amino acid sequence listed in Table 9. For example, the protein can differ at at least one, but no more than two, three, four, five, or six amino acids that are located at least 5, 10, or 15 Angstroms from the protease contacting residues, e.g., as defined by a structural model listed herein. The Kunitz domain can include other features described herein.

Treatments

In still another aspect, the invention features a pharmaceutical composition that includes a ligand described herein, and a pharmaceutically acceptable carrier, e.g., a carrier other than water. The composition can further include another therapeutic agent, e.g., an agent that regulates endothelial cell activity.

In another aspect, the invention features a method of modulating an activity of an ET1 -expressing cell. The method includes: contacting an ET1-expressing cell with a ligand described herein in an amount sufficient to modulate an activity of the ET1-expressing cell. Typically, the ligand inhibits the protease activity of ET1. In another embodiment, the activity of the ET1-expressing cell can be a metabolic, transcriptional, secretory, or translational activity, and the ligand includes, or is associated with, an agent that inhibits the activity. The contacting can occur in vitro or in vivo. The method can include other features described herein. For example, the ligand can include a compound, peptide, Kunitz domain, or protein, that binds, e.g.,. independently binds, to ET1.

In another aspect, the invention features a method of altering the endotheliase activity of an ET1-expressing cell. The method includes: contacting a ligand described herein to the ET1-expressing cell, wherein the ligandprevents binding of the ET1-expressing cell to a substrate, e.g., a vessel basement membrane. For example, the cell is a metastatic cancer cell. The method can include other features described herein.

In another aspect, the invention features a method of altering the endotheliase activity in a subject. The method includes: administering the pharmaceutical composition that includes a ligand or composition described herein to the subject in an amount sufficient to inhibit ET1 activity in at least one tissue of the subject. The ligandcan be administered locally or systemically. In one embodiment, the subject is a mammal, e.g., a human. In one embodiment, the ligand is an antagonist of ET1 and the amount is effective to antagonize ET1 activity.

In one embodiment, the subject has or is at risk for having a neoplasia, e.g., a hyperplasia, a tumor, or a metastatic cancer. In one embodiment, the subject has or is at risk for having a disorder characterized by excess angiogenesis. Exemplary disorders include: rheumatoid arthritis, psoriasis, diabetic retinopathies, ocular disorder such as pterygii recurrence, surgery (e.g., scarring excimer laser surgery and glaucoma filtering surgery), cardiovascular disorders, chronic inflammatory disorders, wound repair, circulatory disorders, crest syndromes, dermatological disorders, and cancers.

In one embodiment, the subject has or is at risk for having an angiogenesis-dependent cancer or tumor. “Angiogenesis-dependent cancers and tumors” are cancers and tumors that require, for their growth (expansion in volume and/or mass), an increase in the number and density of the blood vessels supplying then with blood. In one embodiment, the ligand is an administered in an amount sufficient to cause regression of such cancers and tumors. “Regression” refers to the reduction of tumor mass and size, e.g., a reduction of at least 2, 5, 10, or 25%. In some cases, the regression can be at least 40%, 50%, 60%, 70% or 80%.

In one embodiment, the amount is effective to reduce angiogenesis in the subject, and/or ameliorate at least one symptom of a disorder.

The method can include other features described herein.

In another aspect, the invention features a method of inhibiting proteolysis of an extracellular matrix or vessel basement membrane component and/or structure. The method includes: contacting a tissue or structure (e.g., the vessel basement membrane) with a ligand described herein in an amount sufficient to inhibit the proteolysis of a vessel basement membrane component. The method can include other features described herein.

In one embodiment, the contacting occurs in a subject. In one embodiment, the inhibition of proteolysis reduces angiogenesis. In one embodiment, the subject is identified as a subject requiring a therapy to reduce tumor growth or metastasis.

In another aspect, the invention features a method of altering an activity of a cell (e.g., altering cellular growth, viability, proliferation, metabolism, or adherence or ablating or killing a cell), the method comprising contacting the cell with a ligand described herein in an amount sufficient to alter an activity of a cell. In one embodiment, the cell is a metastatic cancer cell. The method can include other features described herein.

In another aspect, the invention features a method of reducing endotheliase activity in a subject. The method includes: identifying a subject in need of reduced endotheliase activity and administering the pharmaceutical composition that includes a ligand described herein to the subject. In one embodiment, the subject is a mammal (e.g., mouse, human). In one embodiment, the pharmaceutical composition is administered in combination with another treatment or agent selected from anti-cancer and/or anti-angiogenic agents. The method can include other features described herein.

In another aspect, the invention features a method of treating or preventing a disorder characterized by unwanted angiogenesis in a subject. The method includes: administering the pharmaceutical composition that includes a ligand described herein to a subject having the disorder or predisposed to the disorder. The method can include other features described herein. For example, the disorder is a disorder selected from the group consisting of: rheumatoid arthritis, psoriasis, diabetic retinopathies, ocular disorders such as pterygii recurrence, a disorder arising from scarring excimer laser surgery or glaucoma filtering surgery, cardiovascular disorders, chronic inflammatory disorders, wound repair, circulatory disorders, crest syndromes, dermatological disorders, and cancers. The method can include other features described herein.

In another aspect, the invention features a method of increasing ET1 activity in a subject. The method includes administering to a subject an effective amount of an ET1-binding ligand that agonizes ET1 binding activity. The method can be used, e.g., to stimulate angiogenesis, e.g., to aid wound healing, burns, and other disorders which require increased angiogenesis. The ET1-binding ligand can be a compound that includes a peptide.

Detection

In another aspect, the invention features a method of detecting endotheliase in a subject. The method includes: administering a labeled ligand described herein to a subject and detecting the label in the subject. In one embodiment, the detecting includes imaging the subject. The method can include other features described herein.

In another aspect, the invention features a method of detecting endotheliase activity in a sample. The method includes: contacting the sample with a labeled ligand described herein and detecting the label. The method can include other features described herein.

Libraries

In another aspect, the invention features a nucleic acid library that includes a plurality of varied nucleic acids, wherein each nucleic acid of the plurality encodes a protein comprising:

| | |
|---|---|
| C4-X5-X6-X7-X8-X9-X10-C11, | (SEQ ID NO:213) |

wherein X5 is L or I, X6 is S or T, X7 is R or K, X8 is D, X9 is I, L, P, or T, and X10 is P, and at least 5, 10, 50, 10², 10⁴, or 10⁵ unique proteins are represented by the different nucleic acids of the plurality. For example, the library is designed according to FIG. 2, or to one or more varied features depicted in FIG. 2. In one embodiment, the plurality of nucleic acids constitutes at least 10, 25, 30, 50, 70, 80, 90, 95, or 100% of the library. The library can include other features described herein.

In another aspect, the invention features a nucleic acid library that includes a plurality of varied nucleic acids, wherein each nucleic acid of the plurality encodes a protein comprising:

C4-X5-X6-X7-X8-X9-X10-C11,

wherein X5 is K or R, X6 is G, X7 is Y or F, X8 is Y, W, or A, X9 is P, and X10 is D, and at least 5, 10, 50, 10², 10⁴, or 10⁵ unique proteins are encoded by the different nucleic acids of the plurality. For example, the library is designed according to FIG. 3, or to one or more varied features depicted in FIG. 3. In one embodiment, the plurality of nucleic acids constitutes at least 10, 25, 30, 50, 70, 80, 90, 95, or 100% of the library. The library can include other features described herein.

A nucleic acid library described herein can include at least two or three codon positions N-terminal to C4, and/or C-terminal to C11 that are also varied. In one embodiment, the plurality contains between 10⁴ unique coding sequences and 10⁸ unique coding sequences. In one embodiment, no more than eight codons are varied.

The invention also features corresponding protein libraries that include a plurality of varied proteins.

In another aspect, the invention features a method of identifying an ET1 binding protein. The method includes: providing a library described herein, contacting members of the library with a protein that comprises the protease domain of ET1, and identifying one or more members of the library that interact with a protein that comprises the protease domain of ET1. The method can include other features described herein.

In another aspect, the invention features a ligand that specifically binds to ET1 and that competes for an ET1 epitope with a ligand described herein. For example, the ligand can include a compound, peptide, Kunitz domain, or protein that binds, e.g.,. independently binds, to ET1 and competes with an ET1 epitope with a ligand described herein.

In one embodiment, the ligand specifically binds to ET1 and inhibits its proteolytic activity. The ligand could be a Kunitz domain, a peptide, an antibody, or other molecule. Peptide ligands or small proteins, such as Kunitz domains, could be produced recombinantly or chemically synthesized.

In another aspect, the invention provides compositions, e.g., pharmaceutical compositions, which include a pharmaceutically acceptable carrier, excipient or stabilizer, and at least one of the ET1-binding ligands (e.g., a ligand including a compound, peptide, Kunitz domain, or protein that interacts with ET1) described herein. In one embodiment, the composition e.g., the pharmaceutical composition, includes a combination of two or more of the aforesaid ET1-binding ligands. In one embodiment, the composition includes a ligand described herein and another therapeutic compound, e.g., an anti-cancer or an anti-angiogenesis agent.

In another aspect, the invention features a kit that includes an ET1-binding ligand for use alone or in combination with other therapeutic modalities, e.g., a cytotoxic or labeling agent, e.g., a cytotoxic or labeling agent as described herein, along with instructions on how to use the ET1-binding ligand or the combination of such agents to treat, prevent, or detect a lesion, e.g., a disease lesion such as a cancerous lesion.

The invention also features nucleic acid sequences that encode an ET1-binding ligand, e.g., a ligand described herein. In another aspect, the invention features host cells and vectors containing such a nucleic acid, or any other nucleic acid described herein.

In another aspect, the invention features a method of producing an ET1 -binding ligand. The method includes: providing a nucleic acid encoding the ligand and expressing the nucleic acids in a host cell under conditions that allow production of the ligand. In one embodiment, the ligand is secreted. In one embodiment, the host cell is a eukaryotic cell, e.g., a mammalian cell, an insect cell, a yeast cell (e.g., Saccharomyces cerevisiae or Pichia pastoris), or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding a ligand described herein can be expressed in a transgenic animal. In one embodiment, the nucleic acids are placed under the control of a tissue-specific promoter (e.g., a mammary specific promoter) and the ligand is produced in the transgenic animal, such as a transgenic cow, pig, horse, sheep, goat or rodent.

In one embodiment, a ligand includes a plurality of Kunitz domains, at least one of which has a property described herein. For example, each domain of the plurality can have a property described herein. In another example, each Kunitz domain of the plurality is the same. The domains can be arranged in tandem.

The term “polypeptide” refers to a polymer of three or more amino acids linked by peptide bonds. The polypeptide may include one or more unnatural amino acids. Typically, the polypeptide includes only natural amino acids. The term “peptide” refers to a polypeptide that is between three and thirty-two amino acids in length. The term “peptide” can also be used to refer to a sequence of between three and thirty-two amino acids in length that is embedded in a longer amino acid sequence. Accordingly, peptides can be embodiments as proteins whose length is less than or equal to thirty-two amino acids or as components of a larger protein. A “protein” can include one or more polypeptide chains. Accordingly, the term “protein” encompasses polypeptides and peptides. A protein or polypeptide can also include one or more modifications, e.g., a natural modification or an artificial modification. Exemplary modifications include glycosylation, amidation, phosphorylation, PEGylation and so forth.

As used herein, a “Kunitz domain” is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions 5, 14, 30, 38, 51, and 55, and disulfides can form between the cysteines at position 5 and 55, 14 and 38, and 30 and 51), or, if two disulfides are present, they can form between a corresponding subset of cysteines thereof. The spacing between respective cysteines can be within 7,.5, 4, 3, or 2 amino acids of the following spacing: 5 to 55, 14 to 38, and 30 to 51.

Herein, the residues of exemplary Kunitz domains are numbered by reference to the Kunitz domain 1 of LACI-K1 (lipoprotein-associated coagulation inhibitor-domain1) (i.e., residues 1-58, corresponding to Kunitz domain 1 of LACI-K1, see, e.g., Markland et al. (1996) Biochemistry 35:8045-57). Thus, the first cysteine residue of the LACI-K1 Kunitz domain is residue 5 and the last cysteine is residue 55.

Kunitz domains of this invention can be at least 30, 40, 50, 60, 70, 80, or 90% identical to LACI-K1. Other Kunitz domains of this invention are homologous (e.g., at least 30, 40, 50, 60, 70, 80, or 90% identical) to other naturally-occurring Kunitz domains (e.g., a Kunitz domain described herein), particularly to other human Kunitz domains.

In SEQ ID NO:5, listed below, disulfides bonds link at least two of: 5 to 55, 14 to 38,and30to51.

| | |
|---|---|
| X₁-X₂-X₃-X₄-C₅-X₆-X₇-X₈-X₉-X₉ₐ-X₁₀-X₁₁-X₁₂-X₁₃-C₁₄-X₁₅-X₁₆-X₁₇-X₁₈-X₁₉- | (SEQ ID NO:5) |
| X₂₀-X₂₁-X₂₂-X₂₃-X₂₄-X₂₅-X₂₆-X₂₇-X₂₈-X₂₉-X₂₉ₐ-X_{29b}-X_{29c}-C₃₀-X₃₁-X₃₂-X₃₃-X₃₄-X₃₅-X₃₆- | |
| X₃₇-C₃₈-X₃₉-X₄₀-X₄₁-X₄₂-X₄₂ₐ-X_{42b}-X₄₃-X₄₄-X₄₅-X₄₆-X₄₇-X₄₈-X₄₉-X₅₀-C₅₁-X₅₂-X₅₃-X₅₄- | |
| C₅₅-X₅₆-X₅₇-X₅₈. | |

In one embodiment, one or more of residues X₉ₐ, X₂₉ₐ, X_{29b}, X_{29c}, X₄₂ₐ, and X_{42b} are absent. In one embodiment wherein X₉ₐ is absent, X₁₂ is G. In an embodiment in which a particular Kunitz domain framework is used, the ligand includes a Kunitz domain, wherein X₃₃ is F, X₃₇ is G, and X₄₅ is F or Y. See, for example, the pancreatic trypsin inhibitor (Kunitz) family signature in Prosite (Sigrist et al. (2002) “PROSITE: a documented database using patterns and profiles as motif descriptors” Brief Bioinform. 3:265-274).

In an embodiment in which a particular Kunitz domain framework is used, the ligand includes a Kunitz domain that has the following sequence:
M-H—S-F-C₅-A-F—K-A-D-X₁₁-G-X₁₃—C₁₄—X₁₅—X₁₆—X₁₇—X₁₈—X₁₉—R—F—F—F—N—I—F-T-R-Q-C₃₀-E-E-F—X₃₄—Y-G-G-C₃₈—X₃₉—X₄₀—N-Q-N—R—F-E-S-L-E-E-C₅₁—K—K-M-C₅₅-T-R-D-S-A-S—S-A-S-G-D-F-D- (SEQ II) NO:6) wherein X₁₁, X₁₃, X₁₉, X₃₄, and X₃₉ are any amino acid except cysteine, e.g., an amino acid specified herein X₁₅, X₁₇, and X₁₈ are any amino acid except cysteine or proline, e.g. an amino acid specified herein X₁₆ is one of alanine, glycine, glutamic acid, aspartic acid, histidine, or threonine and X₄₀ is glycine or alanine.

A Kunitz domain described herein can have a three-dimensional structure which has an RMSD of less than 4, 3, 2.5, 2, or 1.8 Angstroms relative to a Kunitz domain structural model, e.g., a Kunitz domain in one of the following structural models from the PDB (Protein Data Bank): 1ADZ, 1AVU, 1AVW, 1AVX, 1BA7, IBIK, 1BRC, 1BTH, 1BUN, 1DOD, 1D30, 1DF2, 1EWU, 1EYL, 1FMZ, 1FNO, 1IRH, 1KNT, 1KTH, 1KUN, 1LD5, 1LD6, 1LT2, 1MTN, 1MTS, 1MTU, 1MTV, 1MTW, 1SHP, 1TAW, 1TFX, 1TIE, 1TOC, 1WBC, 2KNT, 2WBC, 4PTI, and 4WBC.

A ligand, compound, peptide, Kunitz domain or protein that “independently binds” to a target molecule binds with a K_{d} of 50 μM or less and is able to bind absent other amino acids sequences to which it may be associated (e.g., covalently attached). For example, the peptide may be part of a protein, but is able to bind to the target molecule in a context where the rest of the protein is absent. A peptide that “does not bind to endotheliase 1 (ET1)” may (a) interact with ET1 with a K_{d} of 50 μM or greater, or (b) not have an experimentally observable interaction with ET1. A description of ET1 can be found, for example, in WO 01/36604. Similar standards are applicable to any independent binding segment of a protein, e.g., a Kunitz domain or any other amino acid subsequence. The invention also includes proteins that bind to a target molecule by the cooperative interaction of two or more separate domains or subsequences.

Binding affinity can be determined by a variety of methods including equilibrium dialysis, equilibrium binding, gel filtration, ELISA, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:
[Bound]=N.[Free]/((1/Kₐ)+[Free])

It is not always necessary to make an exact determination of K_{a,} since sometimes it is sufficient to obtain a quantitative measurement of affinity, e.g., determined using a method such as ELISA or FACS analysis, which is proportional to K_{a,} and thus can be used for comparisons, such as determining whether a higher affinity is, e.g., 2-fold higher. Better binding can be indicated by a greater numerical K_{a,} or a lesser numerical K_{d} than a reference. Unless otherwise noted, binding affinities are determined in phosphate buffered saline at pH7 or the buffer of a binding assay described herein.

An “isolated composition” refers to a composition that is removed from at least 90% of at least one component of a natural sample or a synthetic reaction from which the isolated composition can be obtained. Compositions described herein produced artificially or naturally can be “compositions of at least” a certain degree of purity if the species or population of species of interest is at least 5, 10, 25, 50, 75, 80, 90, 95, 98, or 99% pure on a weight-weight basis.

An “epitope” refers to the site on a target compound that is bound by a ligand, e.g., a polypeptide ligand such as a peptide or Kunitz domain described herein. In the case where the target compound is a protein, for example, an epitope refers to the amino acids that are bound by the ligand.

As used herein, the term “substantially identical” (or “substantially homologous”) is used to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the first antibody.

Sequences similar or homologous (e.g., at least about 85% sequence identity) to the sequences disclosed herein are also part of this application. In some embodiments, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., high stringency hybridization conditions) to the complement of a strand described herein or a strand of a nucleic acid that encodes a protein described herein. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

As used herein, the term “homology” is synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST algorithms (available from the National Center of Biotechnology Information (NCBI), National Institutes of Health, Bethesda Md.); in each case, using the algorithm default or recommended parameters for determining significance of calculated sequence relatedness. The percent similarity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C.; 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. The invention includes a polypeptide encoded by a nucleic acid that hybridizes under one or more of the above conditions to the complement of a coding nucleic acid sequence described herein. For example, the encoded polypeptide can also be structured by one or more disulfide bonds, e.g., as configured in the polypeptide of the coding nucleic acid sequence described herein.

The ligands described herein may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie, et al. (1990) Science 247:1306-1310. In particular, many alterations (e.g., amino acid substitutions, insertions, and/or deletions) are tolerated when the altered positions are distant (e.g., at least 5, 10, or 20 amino acids) from a functional site, e.g., from amino acid positions that interact with a target.

A “conservative amino acid substitution” is one in which the amino acid residue is replaced with an amino acid residue having a similar side chain, e.g., physically or chemically similar. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine); nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the ligand, e.g., a peptide, without abolishing or more preferably, without substantially altering an activity (e.g., binding activity), whereas alteration of an “essential” amino acid residue results in such a change.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular ligands may show a difference, e.g., in specificity or binding, that are statistically significant (e.g., P value <0.05 or 0.02). The details of one or more embodiments of the invention are set forth in the description below. Other features, objects, and advantages of the invention will be apparent from the description and the claims. The contents of all references, pending patent applications and published patents, cited throughout this application are hereby expressly incorporated by reference.

### BRIEF DESCRIPTION OF THE DRAWINGS

FIG. 1 is a graph of ELISA data for binding of exemplary peptides to ET1.

FIG. 2 is a design for a library that includes variations based on: X—X—X—C-(L/I)—(S/T)-(R/K)-D-(I/L/P/T)-P—C—X—X—X (SEQ ID NO:134). A model peptide used in the library screen was AGKMRCLSRDLPCVTHGT (SEQ ID NO:196). The figure also shows two library constructs: the first has the nucleotide sequence SEQ ID NO:200 and the amino acid sequence SEQ ID NO:201; the second has a nucleotide sequence represented by SEQ ID NO:202 and an amino acid sequence of SEQ ID NO:203.

FIG. 3 is a design for a library that includes variations based on: X—X—X—C—(K/R)-G-(Y/F)—Y—P-D-C—X—X—X (SEQ ID NO:135). Two model peptides used in the library screen were AGKHICKGYYPDCGYPGT (SEQ ID NO:197) and AGHWQCKGYAPDCEPWGT (SEQ ID NO:198). The figure also shows two library constructs: the first has the nucleotide sequence SEQ ID NO:204 and the amino acid sequence SEQ ID NO:205; the second has a nucleotide sequence represented by SEQ ID NO:206 and an amino acid sequence of SEQ ID NO:207.

### DETAILED DESCRIPTION

Endotheliase 1 is a serine protease and a member of the endotheliase class of angiogenesis-associated proteases. Inhibition of ET1 may impair or prevent angiogenesis by blocking vessel basement membrane penetration by endothelial cells and subsequent sprout formation. While not intending to be bound by theory, ET1 may participate in angiogenesis by proteolyzing certain substrates, for example, one or more vessel basement membrane components. Accordingly, ET1 inhibitors can be used as antagonists of angiogenesis and are thus potentially valuable therapeutic molecules for the treatment of angiogenesis-dependent diseases such as cancer.

The invention provides, in part, ligands that bind to Endotheliase-1 (ET1), e.g., peptides that bind to ET1 with high affinity and selectivity, compounds that bind with high affinity and selectivity, Kunitz domains that bind to ET1 with high affinity and selectivity and proteins that bind with high affinity and selectivity. The amino acid sequences of exemplary peptides and exemplary Kunitz domains that bind and/or inhibit ET1 can be found below. In general, small peptide inhibitors of other proteolytic enzymes are not common. It is generally believed that unmodified small peptides capable of binding to a protease active site will be hydrolyzed by the enzyme. However, many of the peptides described here are remarkable in that they inhibit ET1 without undergoing detectable proteolytic cleavage by the enzyme.

Endotheliase 1

A description of ET1 can be found, for example, in WO 01/36604 and Lang and Schuller,. “Differential expression of a novel serine protease homologue in squamous cell carcinoma of the head and neck” (2001) Br. J. Cancer 84:237-243.

An exemplary ET1 protein can include the following sequences:

| | |
|---|---|
| MYRPDVVRAR KRVCWEPWVI GLVIFISLIV LAVCIGLTVH YVRYNQKKTY NYYSTLSFTT | (SEQ ID NO:1) |
| DKLYAEFGRE ASNNFTEMSQ RLESMVKNAF YKSPLREEFV KSQVIKFSQQ KHGVLAHMLL | |
| ICRFHSTEDP ETVDKIVQLV LHEKLQDAVG PPKVDPHSVK IKKINKTETD SYLNRCCGTR | |
| RSKTLGQSLR IVGGTEVEEG EWPWQASLQW DGSHRCGATL INATWLVSAA HCFTTYKNPA | |
| RWTASFGVTI KPSKMKRGLR RIIVHEKYKH PSHDYDISLA ELSSPVPYTN AVHRVCLPDA | |
| SYEFQPGDVM FVTGFGALKN DGYSQNHLRQ AQVTLIDATT CNEPQAYNDA ITPRMLCAGS | |
| LEGKTDACQG DSGGPLVSSD ARDIWYLAGI VSWGDECAKP NKPGVYTRVT ALRDWITSKT | |
| GI. | |

An exemplary nucleic acid sequence encoding an ET1 protein can include the following sequences:

| | |
|---|---|
| TGACTTGGATGTAGACCTCGACCTTCACAGGACTCTTCATTGCTGGTTGGCAATGATGTATCG | (SEQ ID NO:2) |
| GCCAGATGTGGTGAGGGCTAGGAAAAGAGTTTGTTGGGAACCCTGGGTTATCGGCCTCGTCAT | |
| CTTCATATCCCTGATTGTCCTGGCAGTGTGCATTGGACTCACGTTCATTATGTGAGATATAAT | |
| CAAAAGAAGACCTACAATTACTATAGCACATTGTCATTTACAACTGACAAACTATATGCTGAG | |
| TTTGGCAGAGAGGCTTCTAACAATTTTACAGAAATGAGCCAGAGACTTGAATCAATGGTGAA | |
| AAATGCATTTTATAAATCTCCATTAAGGGAAGAATTTGTCAAGTCTCAGGTTATCAAGTTCAG | |
| TCAACAGAAGCATGGAGTGTTGGGTCATATGCTGTTGATTTGTAGATTTCACTCTACTGAGGA | |
| TCCTGAAACTGTAGATAAAATTGTTCAACTTGTTTTACATGAAAAGCTGCAAGATGCTGTAGG | |
| ACCCCCTAAAGTAGATCCTCACTCAGTTAAAATTAAAAAAATCAACAAGACAGAAACAGACA | |
| GCTATCTAAACCATTGCTGCGGAACACGAAGAAGTAAAACTCTAGGTCAGAGTCTCAGGATC | |
| GTTGGTGGGACAGAAGTAGAAGAGGGTGAATGGCCCTGGCAGGCTAGCCTGCAGTGGGATGG | |
| GAGTCATCGCTGTGGAGCAACCTTAATTAATGCCACATGGCTTGTGAGTGCTGCTCACTGTTTT | |
| ACAACATATAAGAACCCTGCCAGATGGACTGCTTCCTTTGGAGTAACAATAAAACCTTCGAAA | |
| ATGAAACGGGGTCTCCGGAGAATAATTGTCCATGAAAAATACAAACACCCATCACATGACTA | |
| TGATATTTCTCTTGCAGAGCTTTCTAGCCCTGTTCCCTACACAAAGCAGTACATAGAGTTTGT | |
| CTCCCTGATGCATCCTATGAGTTTCAACCAGGTGATGTGATGTTTGTGACAGGATTTGGAGCA | |
| CTGAAAAATGATGGTTACAGTCAAAATCATCTTCGACAAGCACAGGTGACTCTCATAGACGCT | |
| ACAACTTGCAATGAACCTCAAGCTTACAATGACGCCATAACTCCTAGAATGTTATGTGCTGGC | |
| TCCTTAGAAGGAAAAACAGATGCATGCCAGGGTGACTCTGGAGGACCACTGGTTAGTTCAGA | |
| TGCTAGAGATATCTGGTACCTTGCTGGAATAGTGAGCTGGGGAGATGAATGTGCGAAACCCA | |
| ACAAGCCTGGTGTTTATACTAGAGTTACGGCCTTGCGGGACTGGATTACTTCAAAAACTGGTA | |
| TCTAAGAGAGAAAAGCCTCATGGAACAGATAAC. | |

An exemplary ET1 protease domain can include the following sequence:

| | |
|---|---|
| RIVGGTEVEEGEWPWQASLQWDGSHRCGATLINATWLVSAAHCFTTYKNPARWTASEGVTIKYS | (SEQ ID NO:3) |
| KMKRGLRRIIVHEKYKIPSHDYDISLAELSSPVPYTNAVHRVCLPDASYEFQPGDVMFVTGFGAL | |
| KNDGYSQNHLRQAQVTLIDATTCNEPQAYNDAITPRMLCAGSLEGKTDACQGDSGGPLVSSDAR | |
| DIWYLAGIVSWGDECAKPNKPGVYTRVTALRDWITSKTGI | |

gi|14348558|emb|CAC41266.1|cDNA encoding protease domain of endotheliase 1).

An exemplary nucleic acid that encodes a ET1 protease domain can include the following sequence:

| | |
|---|---|
| AGGATCGTTGGTGGGACAGAAGTAGAAGAGGGTGAATGGCCCTGGCAGGCTAGCCTGCAGTG | (SEQ ID NO:4) |
| GGATGGGAGTCATCGCTGTGGAGCAACCTTAATTAATGCCACATGGCTTGTGAGTGCTGCTCA | |
| CTGTTTTACAACATATAAGAACCCTGCCAGATGGAGTGCTTCCTTTGGAGTAACAATAAAAACC | |
| TTCGAAAATGAAACGGGGTCTCCGGAGAATAATTGTCCATGAAAAATACAAACACCCATCAC | |
| ATGACTATGATATTTCTCTTGCAGAGCTTTCTAGCCCTGTTCCCTACAAATGCAGTACATAG | |
| AGTTTGTCTCCCTGATGCATCCTATGAGTTTCAACCAGGTGATGTGATGTTTGTGACAGGATTT | |
| GGAGCACTGAAAAATGATGGTTACAGTCAAAATCATCTTCGACAAGCACAGGTGACTCTCAT | |
| AGACGCTACAACTTGCAATGAACCTCAAGCTTACAATGACGCCATAACTCCTAGAATGTTATG | |
| TGCTGGCTCCTTAGAAGGAAAAACAGATGCATGCCAGGGTGACTCTGGAGGACCACTGGTTA | |
| GTTCAGATGCTAGAGATATCTGGTACCTTGCTGGAATAGTGAGCTGGGGAGATGAATGTGCGA | |
| AACCCAACAAGCCTGGTGTTTATACTAGAGTTACGGCCTTGCGGGACTGGATTACTTCAAAAA | |
| CTGGTATCTAA, | |

gi|14348557|emb|AX149577.1|Sequence 1 from Patent WO0136604).

An endotheliase protein can include an SEA domain, e.g., including about amino acid 65-107 of SEQ ID NO:1, and a serine protease domain, including about amino acids 191-421 of SEQ ID NO:1. The serine protease domain can include an active site histidine, e.g., at about amino acid 231 of SEQ ID NO:1 and an active site serine at about amino acid 372 of SEQ ID NO:1. An ET1-binding ligand can physically interact with at least one of these features.

In one embodiment, an ET1 protein can be glycosylated, e.g., at a site at about amino acids 74-75, 165-168, and 222-225 of SEQ ID NO:1. In one embodiment, the site at 222-225 is not glycosylated. An ET1-binding ligand can physically interact with at least one of these features. The ET1-binding ligand can bind one or more amino acids of ET1, e.g., by contacting one or more amino acids residues 1-40, 40-80, 80-120, 120-160, 160-200, 200-240, 240-280, 280-320, 320-360, 360-400, or 400 to the carboxy terminus of SEQ ID NO:1.

Display Libraries

A display library can be used to identify ligands, e.g., compounds, peptides, proteins and Kunitz domains, that bind to the ET1. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. In a selection, the polypeptide component of each member of the library is probed with the ET1 and if the polypeptide component binds to the ET1, the display library member is identified, typically by retention on a support. In addition, a display library entity can include more than one polypeptide component, for example, the two polypeptide chains of a Fab.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The polypeptide component is typically covalently linked to a bacteriophage coat protein. The linkage results from translation of a nucleic acid encoding the polypeptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-18230; Hoogenboom et al. (1998) Immunotechnology 4:1-20; Hoogenboom et al. (2000) Immunol. Today 2:371-378; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Rebar et al. (1996) Methods Enzymol. 267:129-49; Hoogenboom et al. (1991) Nucl. Acid Res. 19:4133-4137; and Barbas et al. (1991) Proc. Natl. Acad. Sci. USA 88:7978-7982.

Phage display systems have been developed for filamentous phage (phage fl, fd, and M13) as well as other bacteriophage (e.g. T7 bacteriophage and lambdoid phages see, e.g., Santini (1998) J. Mol. Biol. 282:125-135; Rosenberg et al. (1996) Innovations 6:1-6; Houshmand et al. (1999) Anal. Biochem. 268:363-370). The filamentous phage display systems typically use fusions to a minor coat protein, such as gene III protein,or a major coat protein, such as gene VIII protein. Fusions to other coat proteins such as gene VI protein, gene VII protein, gene IX protein, or domains thereof can also been used (see, e.g., WO 00/71694). In one embodiment, the fusion is to a domain of the gene III protein, e.g., the anchor domain or “stump” (see, e.g., U.S. Pat. No. 5,658,727 for a description of the gene III protein anchor domain). It is also possible to physically associate the protein being displayed to the coat using a non-peptide linkage, e.g., a non-covalent bond or a non-peptide covalent bond. For example, a disulfide bond and/or c-fos and c-jun coiled-coils can be used for physical associations (see, e.g., Crameri et al. (1993) Gene 137:69 and WO 01/05950).

The valency of the polypeptide component can also be controlled. Cloning of the sequence encoding the polypeptide component into the complete phage genome results in multivariant display since all replicates of the gene III protein are fused to the polypeptide component. For reduced valency, a phagemid system can be utilized. In this system, the nucleic acid encoding the polypeptide component fused to gene III is provided on a plasmid, typically less than 7000 nucleotides in length. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the polypeptide component can be grown and harvested using standard phage preparatory methods, e.g., PEG precipitation from growth media.

After selection of individual display phages, the nucleic acid encoding the selected polypeptide components can be recovered by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557. In one embodiment, variegated nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 ; Hanes et al. (2000) Nat. Biotechnol. 18:1287-92; Hanes et al. (2000) Methods Enzymol. 328:404-30; and Schaffitzel et al. (1999) J. Immunol. Methods. 231(1-2):119-35.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Synthetic Diversity. In one embodiment, a display library includes one or more regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion. Other examples include N—N-(TG) or N—N-(T-C) and combinations that exclude stop codons or one or more amino acid-encoding codons.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in Wells et al. (1985) Gene 34:315-323; U.S. Pat. Nos. 4,760,025 and 5,869,644. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encode a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis.

Peptides The binding ligand can include a peptide of 32 amino acids or less that binds to ET1. Some peptides can include one or more disulfide bonds (e.g., exactly one, two, or three). Other peptides, so-called “linear peptides,” are devoid of cysteines. Still others may include an odd number of cysteines (e.g., exactly one cysteine). In one embodiment, the peptides are artificial, i.e., not present in nature or not present in a protein encoded by one or more genomes of interest, e.g., the human genome. Synthetic peptides may have little or no structure in solution (e.g., unstructured), heterogeneous structures (e.g., alternative conformations or “loosely structured), or a singular native structure (e.g., stably folded). Some synthetic peptides adopt a particular structure when bound to a target molecule. Some exemplary synthetic peptides are so-called “cyclic peptides” that have at least a disulfide bond and, for example, a loop of about 4 to 12 non-cysteine residues. Exemplary peptides are less than 28, 24, 20, or 18 amino acids in length.

Peptide sequences that bind a molecular target, e.g., ET1, can be selected from a display library or an array of peptides. After identification, such peptides can be produced synthetically or by recombinant means. The sequences can be incorporated (e.g., inserted, appended, or attached) into longer sequences.

The following are some exemplary phage libraries from which least some of the peptide ligands described herein could be selected. Each library displays a short, variegated exogenous peptide on the surface of M13 phage. The peptide display of five of the libraries was based on a parental domain having a segment of 4, 5, 6, 7, 8, 10, 11, or 12 amino acids, respectively, flanked by cysteine residues. The pairs of cysteines are believed to form stable disulfide bonds, yielding a cyclic display peptide. The cyclic peptides are displayed at the amino terminus of protein III on the surface of the phage. The libraries were designated TN6/7, TN7/4, TN8/9, TN9/4, TN10/10. TN11/1, and TN12/1. Peptides were also selected from a phage library, designated Lin20, with a 20-amino acid linear display.

The TN6/7 library was constructed to display a single cyclic peptide contained in a 12-amino acid template. The TN6/6 library utilized a template sequence of Xaa₁-Xaa₂-Xaa₃-Cys₄-Xaa₅-Xaa₆-Xaa₇-Xaa₈-Cys₉Xaa₁₀-Xaa₁₁-Xaa₁₂, where each variable amino acid position in the amino acid sequence of the template is indicated by a subscript integer. Each variable amino acid position (Xaa) in the template was varied to contain any of the common a-amino acids, except cysteine (Cys).

The TN7/4 library was constructed to display a single cyclic peptide contained in a 13-amino acid template. The TN7/4 library utilized a template sequence of Xaa₁-Xaa₂-Xaa₃-Cys₄-Xaa₅-Xaa₆-Xaa₇-Xaa₈-Xaa₉-Cys₁₀-Xaa₁₁-Xaa₁₂-Xaa₁₃, where each variable amino acid position in the amino acid sequence of the template is indicated by a subscript integer. Each variable amino acid position (Xaa) in the template was varied to contain any of the common a-amino acids, except cysteine (Cys).

The TN8/9 library was constructed to display a single binding loop contained in a 14-amino acid template. The TN8/9 library utilized a template sequence of Xaa₁-Xaa₂-Xaa₃-Cys₄-Xaa₅-Xaa₆-Xaa₇-Xaa₈-Xaa₉-Xaa₁₀-Xaa₁₁-Cys₁₂-Xaa₁₃-Xaa₁₄-Xaa₁₅. Each variable amino acid position (Xaa) in the template were varied to permit any amino acid except cysteine (Cys).

The TN9/4 library was constructed to display a single binding loop contained in a 15-amino acid template. The TN9/4 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Cys₁₂-Xaa₁₃-Xaa₁₄-Xaa₁₅. Each variable amino acid position (Xaa) in the template were varied to permit any amino acid except cysteine (Cys).

The TN10/10 library was constructed to display a single cyclic peptide contained in a 16-amino acid variegated template. The TN10/10 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Cys₁₃-Xaa₁₄-Xaa₁₅-Xaa₁₆, where each variable amino acid position in the amino acid sequence of the template is indicated by a subscript integer. Each variable amino acid position (Xaa) was to permit any amino acid except cysteine (Cys).

The TN11/1 library was constructed to display a single cyclic peptide contained in a 17-amino acid variegated template. The TN11/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Cys₁₄-Xaa₁₅-Xaa₁₆-Xaa₁₇, where each variable amino acid position in the amino acid sequence of the template is indicated by a subscript integer. Each variable amino acid position (Xaa) was to permit any amino acid except cysteine (Cys).

The TN12/1 library was constructed to display a single cyclic peptide contained in an 18-amino acid template. The TN12/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Xaa₁₄-Cys15-Xaa₁₆-Xaa₁₇-Xaa₁₈, where each variable amino acid position in the amino acid sequence of the template is indicated by a subscript integer. The amino acid positions Xaa₁, Xaa₂, Xaa₁₇ and Xaa₁₈ of the template were varied, independently, to permit each amino acid selected from the group of 12 amino acids consisting of Ala, Asp, Phe, Gly, His, Leu, Asn, Pro, Arg, Ser, Trp, and Tyr. The amino acid positions Xaa₃, Xaa₅, Xaa₆, Xaa₇, Xaa₈, Xaa₉, Xaa₁₀, Xaa₁₁, Xaa₁₂, Xaa₁₃, Xaa₁₄, Xaa₁₆, of the template were varied, independently, to permit any amino acid except cysteine (Cys).

The Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and candidates selected, e.g., as described above, for peptides that bind to ET1.

For any particular peptide that includes an intra-molecular disulfide bond, the peptide can be redesigned to replace the disulfide bond with another bond that maintains the geometry of the loop. For example, the distance between the alpha carbons of the first amino acid of the loop (which is C-terminal to the first cysteine of the loop) and the last amino acid of the loop (which is N-terminal to the second cysteine of the loop) can be maintained within 10, 6, 4, or 3 Angstroms of the distance between those alpha carbons in a disulfide bonded loop. In another example, the alpha carbons of the first amino acid of the loop and the last amino acid of the loop are maintained within 15, 12, 10, 8, or 7 inter-atomic bonds of each other. It is also possible to position another amino acid (natural or non-natural) in place of the cysteines, in which case the alpha carbons of these respective replacement amino acids may be within 9, 8, or 6 bonds of each other. Exemplary bonds include C—C, C—N, C—S, O—N, and C—O bonds. Generally, any chemical linker of appropriate length can be used to replace a disulfide bond.

Peptides can also include non-naturally-occurring amino acids and other monomer units that are not found in nature, e.g., a peptoid subunit. One or more of the amino acid units in a peptide can be replaced with another monomer unit to create a region which is other than a peptide-backbone, e.g., to create a peptido-mimetic which preserves the geometry of sidechains, e.g., so that side chains are positioned within an RMSD of less than 5, 3, 2.5, 2.1, 2, or 1.8 of the structure of the original peptide bound to the ET1 when the mimetic is bound to ET1. ( See also, e.g., Patch and Barron (2002) Curr. Opin. Chem. Biol.6:872-877).

Other Exemplary Scaffolds

Other exemplary scaffolds that can be variegated to produce a protein that binds to ET1 can include: extracellular domains (e.g., fibronectin Type III repeats, EGF repeats), protease inhibitors (e.g., Kunitz domains, ecotin, BPTI, and so forth), TPR repeats, trifoil structures, zinc finger domains, DNA-binding proteins, particularly monomeric DNA binding proteins, RNA binding proteins enzymes(e.g., proteases ,particularly inactivated proteases), RNase chaperones(e.g., thioredoxin) heat shock proteins, intracellular signaling domains (such as SH2 and SH3 domains), antibodies (e.g., Fab fragments, single chain Fv molecules (scFV), single domain antibodies, camelid antibodies, and camelized antibodies), T-cell receptors, and MHC proteins.

U.S. Pat. No. 5,223,409 also describes a number of so-called “mini-proteins,” e.g., mini-proteins modeled after α-conotoxins (including variants GI, GII, and MI), mu-(GIIIA, GIIIB, GIIIC), or OMEGA-(GVIA, GVIB, GVIC, GVIIA, GVIIB, MVIIA, MVIIB, etc.) conotoxins.

Methods for producing and using Kunitz domain display library are described, e.g., in U.S. Pat. Nos. 5,223,409; 6,057,287; 6,103,499; and 6,423,498.

Selections from Phage Display Libraries for ET1-Binding Peptides

In an exemplary selection, a phage library is contacted with and allowed to bind the ET1 or a fragment thereof, e.g., a protease domain. To facilitate separation of binders and non-binders in the selection process, it is often convenient to immobilize the target compound on a solid support, although it is also possible to first permit binding to the target compound in solution and then segregate binders from non-binders by coupling the target compound to a support. By way of illustration, when incubated in the presence of the target, phage bearing an ET1-binding moiety form a complex with the ET1 immobilized on a solid support whereas non-binding phage remain in solution and may be washed away with buffer. Bound phage may then be liberated from the ET1 by a number of means, such as changing the buffer to a relatively high acidic or basic pH (e.g., pH 2 or pH 10), changing the ionic strength of the buffer, adding denaturants, or other known means. Alternatively, the bound phage may be recovered by contacting infectable host cells to the solid support.

For example, to identify ET1-binding peptides, ET1 can be adsorbed to a solid surface, such as the plastic surface of wells in a multi-well assay plate. Subsequently, an aliquot of a phage display library is added to a well under appropriate conditions that maintain the structure of the immobilized ET1 and the phage, such as pH 6-7. The phage in the library can display proteins that include a varied peptide or varied Kunitz domain. Phage in the libraries that bind the immobilized ET1 are retained bound to the ET1 adhering to the surface of the well and non-binding phage can be removed. It is also possible to include a blocking agent or competing ligand during the binding of the phage library to the immobilized ET1.

Phage bound to the immobilized ET1 may then be eluted by washing with a buffer solution having a relatively strong acid pH (e.g., pH 2) or an alkaline pH (e.g., pH 8-9). The solutions of recovered phage that are eluted from the ET1 are then neutralized and may, if desired, be pooled as an enriched mixed library population of phage displaying ET1 binding peptides. Alternatively the eluted phage from each library may be kept separate as a library-specific enriched population of ET1 binders. Enriched populations of phage displaying ET1 binding peptides may then be grown up by standard methods for further rounds of selection and/or for analysis of peptide displayed on the phage and/or for sequencing the DNA encoding the displayed binding peptide.

One of many possible alternative selection protocols uses ET1 target molecules that are biotinylated and that can be captured by binding to streptavidin, for example, coated on particles such as magnetic beads.

Recovered phage may then be amplified by infection of bacterial cells, and the selection process may be repeated with the new pool of phage that is now depleted in non-ET1 binders and enriched in ET1 binders. The recovery of even a few binding phage may be sufficient to carry the process to completion. After a few rounds of selection, the gene sequences encoding the binding moieties derived from selected phage clones in the binding pool are determined by conventional methods, revealing the peptide sequence that imparts binding affinity of the phage to the target. An increase in the number of phage recovered after each round of selection and the recovery of closely related sequences indicate that the selection is converging on sequences of the library having a desired characteristic.

After a set of binding polypeptides is identified, the sequence information may be used to design other, secondary libraries, biased for members having improved or additional desired properties. Other types of display libraries can also be used to identify an ET1 binder.

Display technology can also be used to obtain ligands that are specific to particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library.

Iterative Selection. In one preferred embodiment, display library technology is used in an iterative mode. A first display library is used to identify one or more ligands for a target. These identified ligands are then varied using a mutagenesis method to form a second display library. Higher affinity ligands are then selected from the second library, e.g., by using higher stringency or more competitive binding and washing conditions.

In some implementations, the mutagenesis is targeted to regions known or likely to be at the binding interface. Some exemplary mutagenesis techniques include: error-prone PCR (Leung et al. (1989) Technique 1:11-15), recombination, DNA shuffling using random cleavage (Stemmer (1994) Nature 389-391 termed “nucleic acid shuffling”), RACHITT™ (Coco et al. (2001) Nat. Biotech. 19:354), site-directed mutagenesis (Zooler et al. (1987) Nuci. Acids Res. 10:6487-6504), cassette mutagenesis (Reidhaar-Olson (1991) Methods Enzymol. 208:564-586) and incorporation of degenerate oligonucleotides (Griffiths et al. (1994) EMBO J. 13:3245).

In one example of iterative selection, the methods described herein are used to first identify a peptide ligand from a display library that binds a ET1 with at least a minimal binding specificity for a target or a minimal activity, e.g., an equilibrium dissociation constant for binding of less than 50 μM, 1 μM, 500 nM, 200 nM, 100 nM, 50 nM, 5 nM, 500 pM, or 10 pM. The nucleic acid sequence encoding the initial identified protein ligand is used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between polypeptides and their targets, the methods described herein can be used to isolate ligands with a desired kinetic dissociation rate (i.e. reduced) for a binding interaction to ET1.

To select for slow dissociating ligands from a display library, the library is contacted to an immobilized target, e.g., immobilized ET1. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The first solution can have solution conditions that are substantially physiological or that are stringent, e.g., more stringent than physiological. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting and Screening for Specificity. The display library selection and screening methods described herein can include a selection or screening process that discards display library members that bind to a non-target molecule, e.g., a protease other than ET1, e.g., trypsinogen-IV, membrane-type serine proteases-1, -6, -7, urokinase-like plasminogen activator (uPA), trypsin, factor IIa, plasmin (Plm), and/or factor Xa or ET2. In one embodiment, the non-target molecule is an ET1 molecule that has been inactivated, e.g., inactivated by treatment with a covalent inhibitor, e.g., AEBSF.

In one implementation, a so-called “negative selection” step is used to discriminate between the target and related non-target molecules, e.g., molecules that are at least 30, 50, or 70% identical, but less than 98, 95, or 90% identical. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target.

Characterization of ET1 Inhibition

ET1 ligands are screened for binding to ET1 and for inhibition of ET1 proteolytic activity. Peptides can be selected for their potency and selectivity of inhibition of ET1. In one example, ET1 and its substrate are combined under assay conditions permitting reaction of the protease with its substrate. The assay is performed in the absence of the peptide ligand, and in the presence of increasing concentrations of the peptide ligand. The concentration of test ligand at which 50% of the ET1 activity is inhibited by the test ligand is the IC₅₀ value (Inhibitory Concentration) or EC₅₀ (Effective Concentration) value for that ligand. Within a series or group of peptide ligands, those having lower IC₅₀ or EC₅₀ values are considered more potent inhibitors of the ET1 than those ligands having higher IC₅₀ or EC₅₀ values. Preferred ligands can have an IC₅₀ value of 100 nM or less as measured in an in vitro assay for inhibition of ET1 activity.

The ligands also are evaluated for selectivity toward ET1. A test compound is assayed for its potency toward a panel of serine proteases and other enzymes and an IC₅₀ value is determined for each peptide. A ligand that demonstrates a low IC₅₀ value for the ET1 enzyme, and a higher IC₅₀ value for other enzymes within the test panel (e. g., trypsinogen-IV, membrane-type serine proteases-1, -6, -7, urokinase-like plasminogen activator (uPA), trypsin, factor IIa, plasmin (Plm), and/or factor Xa or ET2), is considered to be selective toward ET1. Exemplary ligands may have an IC₅₀ for ET1 that is at least 2, 5, 10, or 100-fold lower than for a non-ET1 protease, e.g., trypsinogen-IV, membrane-type serine proteases-1, -6, -7, urokinase-like plasminogen activator (uPA), trypsin, factor IIa, plasmin (Plm), and/or factor Xa or ET2. Generally, a ligand is deemed highly selective if its IC₅₀ value is at least one order of magnitude less than the next smallest IC₅₀ value measured in the panel of serine proteases and other enzymes.

The ability of test ligandsto act as inhibitors of Endotheliase-1 (ET1) catalytic activity can be assessed using an amidolytic assay. See, for example, WO 01/36604.

Recombinant (rET1) is expressed in Pichia and purified. The enzyme is combined with assay buffer: HBSA (10 mM Hepes, 150 mM sodium chloride, pH 7. 4, 0.1% bovine serum albumin). All reagents can be purchased from Sigma Chemical Co. (St. Louis, Mo.). To set up an assay the following reagents can be combined: 50 microliters of HBSA, 50 microliters of the test ligand, diluted (covering a broad concentration range) in HBSA (or HBSA alone for uninhibited velocity measurement), and 50 microliters of the rET-1 diluted in buffer, yielding a final enzyme concentration of 250 pM. After a 30-minute incubation at ambient temperature, the assay can be initiated by addition of 50 microliters of a substrate, e.g., Spectrozyme tPA (Methylsulfonyl-D-cyclohexyltyrosyl-L-glycyl-L-arginine-p-nitroaniline acetate which can be obtained from American Diagnostica, Inc. (Greenwich, Conn.) and prepared in HBSA) to produce a final reaction volume of 200 microliters and a final substrate concentration of 300 μM. The IC₅₀ can be measured by varying the concentration of the test compound (e.g., a candidate peptide). Reaction velocity can be measured by monitoring the absorbance at 405 nm using a spectrophotometer. The IC₅₀ is the concentration of test ligand that causes a 50% decrease in the initial rate of hydrolysis.

The assay can be used to evaluate, for example, a peptide identified from a-phage display library. In one embodiment, a plurality of peptides is evaluated and ranked based on their IC₅₀ or other kinetic parameter. ET2 can be also assayed using this procedure.

Characterization of Binding Interactions

The binding properties of a ligand that binds ET1 can be readily assessed using various assay formats. For example, the binding property of a ligand can be measured in solution by fluorescence anisotropy, which provides a convenient and accurate method of determining a dissociation constant (K_{d}) of a binding moiety for ET1 or for a particular molecular target. In one such procedure, a binding moiety described herein is labeled with fluorescein. The fluorescein-labeled binding moiety may then be mixed in wells of a multi-well assay plate with various concentrations of ET1. Fluorescence anisotropy measurements are then carried out using a fluorescence polarization plate reader.

ELISA. The binding interaction of a ligand for ET1 can also be analyzed using an ELISA assay. For example, the ligand is contacted to a microtitre plate whose bottom surface has been coated with the target, e.g., a limiting amount of the target. The plate is washed with buffer to remove non-specifically bound ligands. Then the amount of the ligand bound to the plate is determined by probing the plate with an antibody specific to the ligand. The antibody can be linked to an enzyme such as alkaline phosphatase, which produces a colorimetric product when appropriate substrates are provided. In the case of a display library member, the antibody can recognize a region that is constant among all display library members, e.g., for a phage display library member, a major phage coat protein.

Homogeneous Assays. A binding interaction between a ligand and ET1 be analyzed using a homogenous assay, i.e., after all components of the assay are added, additional fluid manipulations are not required. For example, fluorescence resonance energy transfer (FRET) can be used as a homogenous assay (see, for example, Lakowicz et al. , U.S. Pat. No. 5,631,169; Stavrianopoulos, et al. , U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter). By titrating the amount of the first or second binding molecule, a binding curve can be generated to estimate the equilibrium binding constant.

Surface Plasmon Resonance (SPR). The binding interaction of a ligand and ET1 can be analyzed using SPR. For example, after sequencing of a display library member present in a sample, and optionally verified, e.g., by ELISA, the displayed polypeptide can be produced in quantity and assayed for binding the target using SPR. SPR or real-time Biomolecular Interaction Analysis (BIA) detects biospecific interactions in real time, without labeling any of the interactants (e.g., BLAcore). Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which is measured as an indication of real-time reactions between biological molecules. Methods for using SPR are described, for example, in U.S. Pat. No. 5,641,640; Raether (1988) Surface Plasmons Springer Verlag; Sjolander, S. and Urbaniczky, C. (1991) Anal. Chem. 63:2338-2345; Szabo et al. (1995) Curr. Opin. Struct. Biol. 5:699-705.

Information from SPR can be used to provide an accurate and quantitative measure of the equilibrium dissociation constant (K_{d}), and kinetic parameters, including kₒₙ and k_{off}, for the binding of a biomolecule to a target. Such data can be used to compare different biomolecules. For example, proteins selected from a display library can be compared to identify individuals that have high affinity for the target and/or that have a slow k_{off}. This information can also be used to develop a structure-activity relationship (SAR) if the biomolecules are related. For example, if the proteins are all mutated variants of a single parental antibody or a set of known parental antibodies, variant amino acids at given positions can be identified that correlate with particular binding parameters, e.g., high affinity and slow k_{off}.

Additional methods for measuring binding affinities include fluorescence polarization (FP) (see, e.g., U.S. Pat. No. 5,800,989), nuclear magnetic resonance (NMR), and binding titrations (e.g., using fluorescence resonance energy transfer).

High-Throughput Ligand Discovery

One exemplary high-throughput ligand discovery method includes selecting candidates from a phage display library that has a diversity library of at least 10⁷ or 10⁸. Phage are contacted to a target molecule, e.g., immobilized on a magnetic bead. Binding phage are isolated, amplified and reselected in one or more additional cycles. Then individual phage are isolated, e.g., into wells of a microtitre plate, and characterized.

For example, robots can be used to set up two ELISA assays for each individual phage. One assay is for binding to ET1, the other is for binding to ET2. An automated plate reader can evaluate the assays and communicate results to a computer system that stores the results in an accessible format, e.g., in a database, spread sheet, or word processing document. Results are analyzed to identify phage that display a protein that binds to ET1. Results can be further sorted, e.g., by affinity or relative affinity, e.g., to identify ligands that bind with higher affinity to ET1 than to a non-target such as ET2.

In addition, robots can be used to set up proteolysis assays, for example, paired assays for inhibition of ET1 and ET2. Activity in the context of ET1 and ET2 can be compared to generate selectivity data.

Assays for ET1 Binding Ligands

Potential ET1 binding ligands can be further characterized in assays that measure their modulatory activity toward ET1 or fragments thereof in vitro or in vivo. For example, ET1 can be combined with a substrate under assay conditions permitting reaction of the ET1 with the substrate. The assay is performed in the absence of the potential ET1 binding ligand and in the presence of increasing concentrations of the potential ET1 binding ligand. The concentration of ligand at which 50% of the ET1 activity is inhibited by the test compound is the IC₅₀ value (Inhibitory Concentration) or EC₅₀ (Effective Concentration) value for that compound. Within a series or group of test ligands, those having lower IC₅₀ or EC₅₀ values are considered more potent inhibitors of ET1 than those ligands having higher IC₅₀ or EC₅₀ values. Preferred ligands have an IC₅₀ value of 100 nM or less as measured in an in vitro assay for inhibition of ET1 activity.

The ligands can also be evaluated for selectivity toward ET1. For example, a potential ET1 binding ligand can be assayed for its potency toward ET1 and a panel of serine proteases and other enzymes and an IC₅₀ value or EC₅₀ value can be determined for each enzymatic target. In one embodiment, a compound that demonstrates a low IC₅₀ value or EC₅₀ value for the ET1, and a higher IC₅₀ value or EC₅₀ value for other enzymes within the test panel (e. g., trypsinogen-IV, membrane-type serine proteases-1, -6, -7, urokinase-like plasminogen activator (uPA), trypsin, factor IIa, plasmin (Plm), and/or factor Xa or ET2) is considered to be selective toward ET1. In one embodiment, a compound that demonstrates a low IC₅₀ value or EC₅₀ value for the ET1, and a higher IC₅₀ value or EC₅₀ value (e,g., at least 2, 5, 10, 50, or 100-fold higher) for ET2 is considered to be selective toward ET1.

Potential ET1 binding ligands can also be evaluated for their activity in vivo. For example, to evaluate the activity of a ligand to reduce tumor growth through inhibition of endotheliase, the procedures described by Jankun et al. (1997) Canc. Res. 57:559-563 can be employed. Briefly, the ATCC cell lines DU145 and LnCaP are injected into SCID mice. After tumors are established, the mice are administered the test ligand. Tumor volume measurements are taken twice a week for about five weeks. A ligand can be deemed active in this assay if an animal to which the ligand was administered exhibited decreased tumor volume, as compared to animals receiving appropriate control compounds (e.g., non-specific antibody molecules).

To evaluate the ability of a ligand to reduce the occurrence of, or inhibit, metastasis, the procedures described by Kobayashi et al. (1994) Int. J. Canc. 57:727-733d can be employed. Briefly, a murine xenograft selected for high lung colonization potential is injected into C57B1/6 mice i.v. (experimental metastasis) or s.c. into the abdominal wall (spontaneous metastasis). Various concentrations of the ligand to be tested can be admixed with the tumor cells in Matrigel prior to injection. Daily i.p. injections of the test ligand are made either on days 1-6 or days 7-13 after tumor inoculation. The animals are sacrificed about three or four weeks after tumor inoculation, and the lung tumor colonies are counted. Evaluation of the resulting data permits a determination as to efficacy of the test ligand, optimal dosing and route of administration.

The activity of the ligands toward decreasing tumor volume and metastasis can be evaluated in the model described in Rabbani et al. (1995) Int. J. Cancer 63:840-845. See also Xing et al. (1997) Canc. Res. 57:3585-3593. There, Mat LyLu tumor cells were injected into the flank of Copenhagen rats. The animals were implanted with osmotic minipumps to continuously administer various doses of test ligand for up to three weeks. The tumor mass and volume of experimental and control animals were evaluated during the experiment, as were metastatic growths. Evaluation of the resulting data permits a determination as to efficacy of the test ligand, optimal dosing, and route of administration. Some of these authors described a related protocol in Xing et al. (1997) Canc. Res., 57:3585-3593.

To evaluate the inhibitory activity of a ligand toward neovascularization, a rabbit cornea neovascularization model can be employed. See, e.g., Avery et al. (1990) Arch. Ophthalmol., 108:1474-1475. In this model, New Zealand albino rabbits are anesthetized. A central corneal incision is made, forming a radial corneal pocket. A slow release prostaglandin pellet is placed in the pocket to induce neovascularization. The test ligand is administered i.p. for five days, then the animals are sacrificed. The effect of the test ligand is evaluated by review of periodic photographs taken of the limbus, which can be used to calculate the area of neovascular response and, therefore, limbal neovascularization. A decreased area of neovascularization as compared with appropriate controls indicates the test ligand was effective at decreasing or inhibiting neovascularization.

An exemplary angiogenesis model used to evaluate the effect of a test ligand in preventing angiogenesis is described by Min et al. (1996) Canc. Res., 56:2428-2433 (1996). In this model, C57BL6 mice receive subcutaneous injections of a Matrigel mixture containing bFGF, as the angiogenesis-inducing agent, with and without the test ligand. After five days, the animals are sacrificed and the Matrigel plugs, in which neovascularization can be visualized, are photographed. An experimental animal receiving Matrigel and an effective dose of test ligand will exhibit less vascularization than a control animal or an experimental animal receiving a less-or non-effective does of ligand.

An in vivo system designed to test ligands for their ability to limit the spread of primary tumors is described by Crowley et al., Proc. Natl. Acad. Sci., 90:5021-5025 (1993). Nude mice are injected with tumor cells (PC3) engineered to express CAT (chloramphenicol acetyltransferase). Ligands to be tested for their ability to decrease tumor size and/or metastases are administered to the animals, and subsequent measurements of tumor size and/or metastatic growths are made. In addition, the level of CAT detected in various organs provides an indication of the ability of the test ligand to inhibit metastasis; detection of less CAT in tissues of a treated animal versus a control animal indicates less CAT-expressing cells have migrated to that tissue or have propagated within that tissue.

In vivo experimental modes designed to evaluate the inhibitory potential of test serine protease inhibitors, using a tumor cell line F311, are described by Alonso et al. (1996) Breast Canc. Res. Treat. 40:209-223. This group describes in vivo studies for toxicity determination, tumor growth, invasiveness, spontaneous metastasis, experimental lung metastasis, and an angiogenesis assay.

The CAM model (chick embryo chorioallantoic membrane model), first described by L. Ossowski ((1998) J. Cell. Biol. 107:2437-2445), provides another method for evaluating the activity of a test ligand. In the CAM model, tumor cells invade through the chorioallantoic membrane. A test ligand that modulates this process can cause less or no invasion of the tumor cells through the membrane. Thus, the CAM assay is performed with CAM and tumor cells in the presence and absence of various concentrations of test ligand. The invasiveness of tumor cells is measured under such conditions to provide an indication of the compound's inhibitory activity. A ligand having inhibitory activity correlates with less tumor invasion.

The CAM model is also used in to assay angiogenesis (i.e., effect on formation of new blood vessels (Brooks et al. (1999) Methods in Molecular Biology 129:257-269 ). According to this model, a filter disc containing an angiogenesis inducer, such as basic fibroblast growth factor (bFGF) is placed onto the CAM. Diffusion of the cytokine into the CAM induces local angiogenesis, which may be measured in several ways such as by counting the number of blood vessel branch points within the CAM directly below the filter disc. The ability of identified ligands to inhibit cytokine-induced angiogenesis can be tested using this model. A test ligand can either be added to the filter disc that contains the angiogenesis inducer, be placed directly on the membrane or be administered systemically. The extent of new blood vessel formation in the presence and/or absence of test ligand can be compared using this model. The formation of fewer new blood vessels in the presence of a test ligand would be indicative of anti-angiogenesis activity.

Endothelial cell proliferation. A candidate ET1-binding ligand can be tested for endothelial cell proliferation inhibiting activity using a biological activity assay such as the bovine capillary endothelial cell proliferation assay, the chick CAM assay, the mouse corneal assay, and assays that evaluate the effect of the ligand on implanted tumors. The chick CAM assay is described, e.g., by O'Reilly, et al. in “Angiogenic Regulation of Metastatic Growth” (1994)Cell79:315-328. Briefly, three day old chicken embryos with intact yolks are separated from the egg and placed in a petri dish. After three days of incubation a methylcellulose disc containing the ligand to be tested is applied to the CAM of individual embryos. After 48 hours of incubation, the embryos and CAMs are observed to determine whether endothelial growth has been inhibited. The mouse corneal assay involves implanting a growth factor-containing pellet, along with another pellet containing the suspected endothelial growth inhibitor, in the cornea of a mouse and observing the pattern of capillaries that are elaborated in the cornea.

Angiogenesis. Angiogenesis may be assayed, e.g., using various human endothelial cell systems, such as umbilical vein, coronary artery, or dermal cells. Suitable assays include Alamar Blue based assays (available from Biosource International) to measure proliferation migration assays using fluorescent molecules, such as the use of Becton Dickinson Falcon HTS FluoroBlock cell culture inserts to measure migration of cells through membranes in presence or absence of angiogenesis enhancer or suppressors and tubule formation assays based on the formation of tubular structures by endothelial cells on Matrigel™ (Becton Dickinson).

Cell adhesion. Cell adhesion assays measure adhesion of cells to purified adhesion proteins or adhesion of cells to each other, in presence or absence of candidate ET1-binding ligands. Cell-protein adhesion assays measure the ability of agents to modulate the adhesion of cells to purified proteins. For example, recombinant proteins are produced, diluted to 2.5 mg/mL in PBS, and used to coat the wells of a microtiter plate. The wells used for negative control are not coated. Coated wells are then washed, blocked with 1% BSA, and washed again. Ligands are diluted to 2× final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell Adhesion. Cell-cell adhesion assays can be used to measure the ability of candidate ET1-binding ligands to modulate binding of cells to each other. These assays can use cells that naturally or recombinantly express an adhesion protein of choice. In an exemplary assay, cells expressing the cell adhesion protein are plated in wells of a multiwell plate together with other cells (either more of the same cell type, or another type of cell to which the cells adhere). The cells that can adhere are labeled with a membrane-permeable fluorescent dye, such as BCECF, and allowed to adhere to the monolayers in the presence of candidate ligands. Unbound cells are washed off and bound cells are detected using a fluorescence plate reader. High-throughput cell adhesion assays have also been described. See, e.g., Falsey J R et al. (2001) Bioconjug. Chem. 12:346-53.

Tubulogenesis. Tubulogenesis assays can be used to monitor the ability of cultured cells, generally endothelial cells, to form tubular structures on a matrix substrate, which generally simulates the environment of the extracellular matrix. Exemplary substrates include Matrigel™ (Becton Dickinson), an extract of basement membrane proteins containing laminin, collagen IV, and heparin sulfate proteoglycan, which is liquid at 4° C. and forms a solid gel at 37° C. Other suitable matrices comprise extracellular components such as collagen, fibronectin, and/or fibrin. Cells are contacted with a test ligand, and their ability to form tubules is detected by imaging. Tubules can generally be detected after an overnight incubation with stimuli, but longer or shorter time frames may also be used. Tube formation assays are well known in the art (e.g., Jones M K et al. (1999) Nat. Med. 5:1418-1423). These assays have traditionally involved stimulation with serum or with the growth factors FGF or VEGF. In one embodiment, the assay is performed with cells cultured in serum free medium. In one embodiment, the assay is performed in the presence of one or more pro-angiogenic agents, e.g., inflammatory angiogenic factors, such as TNF-α, FGF, VEGF, phorbol myristate acetate (PMA), and TNF-alpha.

Cell Migration. An exemplary assay for endothelial cell migration is the human microvascular endothelial (HMVEC) migration assay. See, e.g., Tolsma et al. (1993) J. Cell Biol. 122:497-511. Migration assays are known in the art (e.g., Paik J. H. et al. (2001) J. Biol. Chem. 276:11830-11837). In one example, cultured endothelial cells are seeded onto a matrix-coated porous lamina, with pore sizes generally smaller than typical cell size. The lamina is typically a membrane, such as the transwell polycarbonate membrane (Corning Costar Corporation, Cambridge, Mass.), and is generally part of an upper chamber that is in fluid contact with a lower chamber containing pro-angiogenic stimuli. Migration is generally assayed after an overnight incubation with stimuli, but longer or shorter time frames may also be used. Migration is assessed as the number of cells that crossed the lamina, and may be detected by staining cells with hemotoxylin solution (VWR Scientific) or by any other method for determining cell number. In another exemplary set up, cells are fluorescently labeled and migration is detected using fluorescent readings, for instance using the Falcon HTS FluoroBlok (Becton Dickinson). While some migration is observed in the absence of stimulus, migration is greatly increased in response to pro-angiogenic factors. The assay can be used to test the effect of a ET1-binding ligand on endothelial cell migration.

Sprouting assay. An exemplary sprouting assay is a three-dimensional in vitro angiogenesis assay that uses a cell-number defined spheroid aggregation of endothelial cells (“spheroid”), embedded in a collagen or fibrin gel-based matrix. The spheroid can serve as a starting point for the sprouting of capillary-like structures by invasion into the extracellular matrix (termed “cell sprouting”) and the subsequent formation of complex anastomosing networks (Korff and Augustin (1999) J. Cell Sci. 112:3249-58). In an exemplary experimental set-up, spheroids are prepared by pipetting about 400 human umbilical vein endothelial cells into individual wells of nonadhesive 96-well plates to allow overnight spheroidal aggregation (Korff and Augustin (1998) J. Cell Biol. 143:1341-52). Spheroids are harvested and seeded in 900 μl of methocel-collagen solution and pipetted into individual wells of a 24 well plate to allow collagen gel polymerization. Test ligands are added after 30 min by pipetting 100 μl of 10-fold concentrated working dilution of the test ligands on top of the gel. Plates are incubated at 37° C. for 24 h. Dishes are fixed at the end of the experimental incubation period by addition of paraformaldehyde. Sprouting intensity of endothelial cells can be quantitated by an automated image analysis system to determine the cumulative sprout length per spheroid.

An exemplary in vitro assay for vessel basement membrane degradation is described in Jensen et al. (1986) Thromb. Res. 44:47-53. ET1 can be used in place of trypsin in the presence or absence of a test ligand, e.g., a ET1-binding ligand that is the subject of evaluation. An exemplary in vivo assay for vessel basement membrane degradation is described in Shipley (1996) Proc. Natl. Acad. Sci. USA 93:3942-3946.

In some embodiments, a ET1-binding ligand has a statistically significant effect (e.g., P<0.05 or P<0.002) on an assay described herein, e.g., a cellular assay described herein.

Ligand Production

Recombinant production of polypeptides. Standard recombinant nucleic acid methods can be used to express a polypeptide component of a ligand described herein (e.g., a polypeptide that includes a Kunitz domain). Generally, a nucleic acid sequence encoding the polypeptide is cloned into a nucleic acid expression vector. If the polypeptide is sufficiently small, e.g., the protein is a peptide of less than 50 amino acids, the protein can also be synthesized using automated organic synthetic methods.

The expression vector for expressing the polypeptide can include a segment encoding the polypeptide and regulatory sequences, for example, a promoter, operably linked to the coding segment. Suitable vectors and promoters are known to those of skill in the art and are commercially available for generating recombinant constructs. See, for example, the techniques described in Sambrook & Russell (2001) Molecular Cloning: A Laboratory Manual, 3^{rd} Edition, Cold Spring Harbor Laboratory, N.Y. and Ausubel et al.(1989) Current Protocols in Molecular Biology Greene Publishing Associates and Wiley Interscience, N.Y.

The vector can be used to express the protein in a host cell. The host cell can be a higher eukaryotic host cell, such as a mammalian cell, a lower eukaryotic host cell, such as a yeast cell, or the host cell can be a prokaryotic cell, such as a bacterial cell. Exemplary hosts include eukaryotic hosts such as HeLa cells, CV-1 cell, COS cells, and Sf9 cells, as well as prokaryotic host such as E. coli and B. subtilis.

Scopes (1994) Protein Purification: Principles and Practice, New York:Springer-Verlag and other texts provide a number of general methods for purifying recombinant (and non-recombinant) proteins.

Synthetic production of peptides. The polypeptide component of a compound can also be produced by synthetic means. See, e.g., Merrifield (1963) J. Am. Chem. Soc. 85:2149. For example, the molecular weight of synthetic peptides or peptide mimetics can be from about 250 to about 8,0000 Daltons.

Pharmaceutical Compositions

In another aspect, the present invention provides compositions, e.g., pharmaceutically acceptable compositions, which include an ET1-binding ligand, e.g., a ligand that includes a compound, peptide, protein, or Kunitz domain that binds to ET1 or a ligand described herein, formulated together with a pharmaceutically acceptable carrier. As used herein, “pharmaceutical compositions” encompass labeled ligands for in vivo imaging as well as therapeutic compositions.

As used herein, “pharmaceutically acceptable carrier” includes any and all solvents, dispersion media, coatings, antibacterial and antifingal agents, isotonic and absorption delaying agents, and the like that are physiologically compatible. Preferably, the carrier is suitable for intravenous, intramuscular, subcutaneous, parenteral, spinal, or epidermal administration (e.g., by injection or infusion). Depending on the route of administration, the ligand , may be coated in a material to protect the ligand from the action of acids and other natural conditions that may inactivate the ligand.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfiric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine and the like.

In one embodiment, an ET1-binding ligand described herein can be formulated for sustained release. For example, the ligand can be encapsulated in a matrix, e.g., a lipid-protein-sugar matrix for delivery to an individual. The encapsulated ligand can be formed into small particles, in a size ranging from 5 micrometers to 50 nanometers. The lipid-protein-sugar particles (LPSPs) typically include a surfactant or phospholipid or similar hydrophic or amphiphilic molecule, a protein, a simple and/or complex sugar, and the ET1-binding ligand. In one example, the lipid is dipalmitoylphosphatidylcholine (DPPC), the protein is albumin, and the sugar is lactose. In another example, a synthetic polymer is substituted for at least one of the components of the lipid-protein-sugar particle, e.g., the lipid, protein, and/or sugar. The compounds used to create LPSPs can be naturally occurring and therefore have improved biocompatibility. The particles may be prepared using techniques known in the art including spray drying. See, e.g., U.S. Published application 2002-0150621.

The compositions of this invention may be in a variety of forms. These include, for example, liquid, semi-solid, and solid dosage forms, liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the ET1-binding ligand is administered by intravenous infusion or injection. In another preferred embodiment, the ET1 -binding ligand is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrastemal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot #7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filter sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The ET1-binding ligands can be administered by a variety of methods known in the art, although for many applications, the preferred route/mode of administration is intravenous injection or infusion. For example, for therapeutic applications, the ET1-binding ligand can be administered by intravenous infusion at a rate of less than 30, 20, 10, 5, 3, 1, or 0.1 mg/min to reach a dose of about 1 to 100 mg/m², 7 to 25 mg/m², or 0.5 to 15 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound described herein by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds can be formulated to ensure proper distribution in vivo. For example, the blood-brain barrier (BBB) excludes many highly hydrophilic compounds. To ensure that the therapeutic compounds can cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhancing targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation or other subject parameter (e.g., weight of the subject). It is especially advantageous to formulate parenteral compositions in dosage unit form for ease of administration and uniformity of dosage. “Dosage unit form” as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active ligand and the particular therapeutic effect to be achieved, and (b) the limitations inherent in the art of compounding such an active ligand for the treatment of sensitivity in individuals.

Dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

A pharmaceutical compositions may include a “therapeutically effective amount” or a “prophylactically effective amount” of an ET1-binding ligand described herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition are outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a ligand to inhibit a measurable parameter, e.g., cancer progression or growth, can be evaluated in an animal model system predictive of efficacy in human tumors. Alternatively, this property of a composition can be evaluated by examining the ability of the ligand to inhibit a measurable parameter, such inhibition being measured in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Kits can be prepared that include a ligand that binds to ET1 and instructions for use, e.g., treatment, prophylactic, or diagnostic use. In one embodiment, the instructions for diagnostic applications include the use of the ET1-binding ligand (e.g., antibody or antigen-binding fragment thereof, or other polypeptide or peptide) to detect ET1, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional ET1-binding ligands, formulated as appropriate, in one or more separate pharmaceutical preparations.

Stabilization and Retention

In one embodiment, an ET1-binding ligand is physically associated with a moiety that improves its stabilization and/or retention in circulation, e.g., in blood, serum, lymph, or other tissues.

For example, an ET1-binding ligand can be associated with a polymer, e.g., a substantially non-antigenic polymer, such as a polyalkylene oxide or polyethylene oxide. Suitable polymers will vary substantially by weight. Polymers having molecular number average weights ranging from about 200 to about 35,000 can be used. Molecular weights of from about 1,000 to about 15,000 are preferred and 2,000 to about 12,500 are particularly preferred.

For example, an ET1-binding ligand can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics), polymethacrylates carbomers, branched or unbranched polysaccharides which comprise the saccharide monomers D-mannose, D- and L-galactose, fucose, fructose, D-xylose, L-arabinose, D-glucuronic acid, sialic acid, D-galacturonic acid, D-maimuronic acid (e.g., polymannuronic acid or alginic acid), D-glucosamine, D-galactosamine, D-glucose, and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g., hyaluronic acid, polymers of sugar alcohols such as polysorbitol and polymannitol, heparin, or heparon.

Other compounds can also be attached to the same polymer, e.g., a cytotoxin, a label, or another targeting agent, e.g., another ET1-binding ligand or an unrelated ligand. Mono-activated, alkoxy-terminated polyalkylene oxides (PAO's), e.g., monomethoxy-terminated polyethylene glycols (mPEG's), C₁₋₄ alkyl-terminated polymers, and bis-activated polyethylene oxides (glycols) can be used for crosslinking. See, e.g., U.S. Pat. No. 5,951,974.

In one embodiment, the polymer prior to cross-linking need not be, but preferably is, water soluble. Generally, after crosslinking, the product is water soluble, e.g., exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion or injection if the conjugate is intended to be administered by such routes.

In one embodiment, the polymer contains only a single group which is reactive. This helps to avoid cross-linking of protein molecules. It is within the scope herein to maximize reaction conditions to reduce cross-linking or to purify the reaction products through gel filtration or ion exchange chromatography to recover substantially homogenous derivatives. In other embodiments, the polymer contains two or more reactive groups for the purpose of linking multiple ligands to the polymer backbone. Again, gel filtration or ion exchange chromatography can be used to recover the desired derivative in substantially homogeneous form.

The molecular weight of the polymer can range up to about 500,000 Da, and preferably is at least about 20,000 Da, or at least about 30,000 Da, or at least about 40,000 Da. The molecular weight chosen can depend upon the effective size of the conjugate to be achieved, the nature (e.g. structure, such as linear or branched) of the polymer, and the degree of derivatization.

The covalent crosslink can be used to attach an ET1-binding ligand to a polymer, for example, crosslinking to the N-terminal amino group and epsilon amino groups found on lysine residues, as well as other amino, imino, carboxyl, sulfhydryl, hydroxyl or other hydrophilic groups. The polymer may be covalently bonded directly to the ET1-binding ligand without the use of a multifunctional (ordinarily bifunctional) crosslinking agent. Covalent binding to amino groups is accomplished by known chemistries based upon cyanuric chloride, carbonyl diimidazole, aldehyde reactive groups (PEG alkoxide plus diethyl acetal of bromoacetaldehyde PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG). Carboxyl groups can be derivatized by coupling PEG-amine using carbodiimide. Sulfhydryl groups can be derivatized by coupling to maleimido-substituted PEG (e.g., alkoxy-PEG amine plus sulfosuccinimidyl 4-(N-maleimidomethyl)cyclohexane-l-carboxylate) WO 97/10847 or PEG-maleimide, commercially available from Shearwater Polymers, Inc., Huntsville, Ala.). Alternatively, free amino groups on the ligand (e.g., epsilon amino groups on lysine residues) can be thiolated with 2-imino-thiolane (Traut's reagent) and then coupled to maleimide-containing derivatives of PEG, e.g., as described in Pedley et al. (1994) Br. J. Cancer 70:1126-1130.

Functionalized PEG polymers that can be attached to an ET1-binding ligand are available, e.g., from Shearwater Polymers, Inc. (Huntsville, Ala.). Such commercially available PEG derivatives include, e.g., amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives may vary depending on the ET1-binding ligand, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer.

The conjugates of an ET1-binding ligand and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

Treatments

Ligands that bind to ET1, e.g., peptide ligands and Kunitz domains described herein, have therapeutic and prophylactic utilities. For example, these ligands can be administered to cells in culture, e.g. in vitro or ex vivo, or in a subject, e.g., in vivo, to treat, prevent, and/or diagnose a variety of disorders, such as cancers, e.g., tumors and other metastatic cancers.

As used herein, the term “treat” or “treatment” is defined as the application or administration of an ET1-binding ligand, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve, or affect the disorder, the symptoms of the disorder or the predisposition toward the disorder. “Treating a cell” refers to the activation, inhibition, ablation, or killing of a cell in vitro or in vivo, or otherwise affecting the capacity of a cell, e.g., an aberrant cell, to mediate a disorder, e.g., a disorder as described herein (e.g., a cancerous disorder). In one embodiment, “treating a cell” refers to a reduction in the activity and/or proliferation of a cell, e.g., a hyperproliferative cell. Such reduction does not necessarily indicate a total elimination of the cell, but a reduction, e.g., a statistically significant reduction, in the activity or the number of the cell.

As used herein, an amount of an ET1-binding ligand effective to treat a disorder, or a “therapeutically effective amount” refers to an amount of the ligand which is effective, upon single or multiple dose administration to a subject, in treating a cell, e.g., a cancer cell (e.g., a ET1-expressing tissue or cell), or in curing, alleviating, relieving, or improving a subject with a disorder as described herein beyond that expected in the absence of such treatment. As used herein, “inhibiting the growth” of the neoplasm refers to slowing, interrupting, arresting, or stopping its growth and metastases and does not necessarily indicate a total elimination of the neoplastic growth.

As used herein, an amount of an ET1-binding ligand effective to prevent a disorder, or a “prophylactically effective amount” of the ligand refers to an amount of an ET1-binding ligand, e.g., an ET1 ligand described herein, which is effective, upon single- or multiple-dose administration to the subject, in preventing or delaying the occurrence of the onset or recurrence of a disorder, e.g., a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the ET1 -expressing hyperproliferative cells” means that the rate of growth of the cells will be different, e.g., statistically significantly different, from the untreated cells.

As used herein, the term “subject” is intended to include human and non-human animals. Preferred human animals include a human patient having a disorder characterized by abnormal cell proliferation or cell differentiation. The term “non-human animals” includes all vertebrates, e.g., non-mammals (such as chickens, amphibians, reptiles) and mammals, such as non-human primates, sheep, dog, cow, pig, etc.

In one embodiment, the subject is a human subject. A protein ligand of the invention can be administered to a human subject for therapeutic purposes (discussed further below). Moreover, an ET1-binding ligand can be administered to a non-human mammal (e.g., a primate, pig or mouse) expressing the ET1-like antigen to which the ligand binds for veterinary purposes or as an animal model of human disease. Regarding the latter, such animal models may be useful for evaluating the therapeutic efficacy of the ligand (e.g., testing of dosages and time courses of administration).

In one embodiment, the invention provides a method of treating (e.g., inhibiting or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion such as a cell found in renal, urothelial, colonic, rectal, pulmonary, breast or hepatic, cancers and/or metastases). Methods can include the steps of contacting the cell with an ET1-binding ligand, e.g., an ET1-binding peptide described herein, an ET1-binding compound, an ET1-binding protein, or an ET1-binding Kunitz domain described herein, in an amount sufficient to treat, e.g., inhibit an activity of the cell (e.g., an undesirable activity of the cell) or kill the cell.

The subject method can be used on cells in culture, e.g. in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding the ET1-binding ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the ET1-binding ligand to the subject under conditions effective to permit both binding of the ligand to the cell and the treating, e.g., the killing the cell or inhibiting an undesirable activity of the cell.

An ET1-binding ligand can be used to reduce angiogenesis (e.g., uncontrolled or unwanted angiogenesis)—such as angiogenesis associated with vascular malformations and cardiovascular disorders (e.g., atherosclerosis, restenosis, and arteriovenous malformations), chronic inflammatory diseases (e.g., diabetes mellitus, inflammatory bowel disease, psoriasis, and rheumatoid arthritis), aberrant wound repairs (e.g., those that are observed following excimer laser eye surgery), circulatory disorders (e.g., Raynaud's phenomenon), crest syndromes (e.g., calcinosis, esophageal and dyomotiloty), dermatological disorders (e.g., Port-wine stains, arterial ulcers, systemic vasculitis and scleroderma), or ocular disorders (e.g., blindness caused by neovascular disease, neovascular glaucoma, comeal neovascularization, trachoma, diabetic retinopathy and myopic degeneration). See, e.g., Carmeliet and Jain (2000) Nature 407: 249-257.

An ET1-binding ligand can be used to treat a cancer. As used herein, the terms “cancer”, “hyperproliferative”, “malignant”, and “neoplastic” are used interchangeably, and refer to those cells in an abnormal state or condition characterized by rapid proliferation or neoplasia. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g., to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant, or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary, as well as adenocarcinomas which include malignancies such as most colon cancers, rectal cancer, renal-cell carcinoma, liver cancer, non-small cell carcinoma of the lung, cancer of the small intestine, and so forth. Metastatic lesions of the aforementioned cancers can also be treated or prevented using the methods and compositions described herein.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon, and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97). Lymphoid malignancies which may be treated by the subject method include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF), and Hodgkin's disease.

Methods of administering ET1-binding ligands are described in “Pharmaceutical Compositions”. Suitable dosages of the molecules used will depend on the age and weight of the subject and the particular drug used. The ligands can be used as competitive agents to inhibit, or reduce an undesirable interaction, e.g., between a natural or pathological agent and the ET1.

In one embodiment, the ET1-binding ligands are used to inhibit at least one activity of or kill cancerous cells and normal, benign hyperplastic, and cancerous cells in vivo. The ligands can be used by themselves or conjugated to an agent, e.g., a cytotoxic drug, radioisotope. This method includes: administering the ligand alone or attached to a cytotoxic drug to a subject requiring such treatment.

An ET1-binding ligand can be used to treat any disease associated with abnormal angiogenesis, e.g., not only cancer and proliferative disorders. For example, protracted angiogenesis is observed also in arthritis, psoriasis, chronic inflammation, scleroderma, hemangioma, retrolental fibroplasia, and abnormal capillary proliferation in hemophiliac joints, prolonged menstruation and bleeding, and other disorders of the female reproductive system. In many of these diseases, unrestrained new capillary growth itself contributes to the disease process.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. The term “cytotoxic agent” also encompasses “anti-cancer” or “anti-tumor” agents, e.g., agents that inhibit the development or progression of a neoplasm, particularly a solid tumor, a soft tissue tumor, or a metastatic lesion. The term “cytotoxic” includes, but is not limited to, cell killing. For example, the term encompasses inhibition of an undesirable cellular activity.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, vincristine, vinblastine, vindesine, vinorelbin, taxotere topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol agents acting via other mechanisms of action, e.g., dihydrolenperone, spiromustine, and desipeptide biological response modifiers, e.g., to enhance anti-tumor responses, such as interferon apoptotic agents, such as actinomycin D and anti-hormones, for example anti-estrogens such as tamoxifen or, for example, antiandrogens such as 4′-cyano-3-(4-fluorophenylsulphonyl)-2-hydroxy-2-methyl-3′-(trifluoromethyl) propionanilide. In one embodiment, the agent is a maytansinoid.

Since the ET1-binding ligands recognize tissues undergoing remodeling and angiogenesis, e.g., cancerous tissues, cells in such tissues to which the ligands are directed can be destroyed or inhibited. Alternatively, the ligands bind to cells in the vicinity of the cancerous cells and kill them, thus indirectly attacking the cancerous cells which may rely on surrounding cells for nutrients, growth signals and so forth. Thus, the ET1-binding ligands (e.g., modified with a cytotoxin) can selectively kill or ablate cells in cancerous tissue (including the cancerous cells themselves).

In one embodiment, an ET1-binding ligand can recognize a normal, endothelial cells. In an embodiment, an ET1-binding ligand binds to cells in the vicinity of cancerous cells. The ligands can inhibit the growth of and/or kill these cells. In this manner, the ligands may indirectly attack the cancerous cells which may rely on surrounding cells for nutrients, growth signals, and so forth. Thus, the ET1-binding ligands (e.g., modified with a cytotoxin) can selectively target cells in cancerous tissue (including the cancerous cells themselves).

The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters, as described herein.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in W084/03508 and W085/03508. Examples of cytotoxic moieties that can be conjugated to the ligands include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a polypeptide component thereof) and the cytotoxin (or a polypeptide component thereof) as translational fusions. The recombinant nucleic acid is then expressed, e.g., in cells and the encoded fusion polypeptide isolated.

Procedures for conjugating protein ligands (e.g., antibodies) with the cytotoxic agents have been previously described. Procedures for conjugating chlorambucil with antibodies are described by Flechner (1973) Eur. J. Cancer 9:741-745; Ghose et al. (1972) Br. Med. J. 3:495-499; and Szekerke, et al. (1972) Neoplasma 19:211-215. Procedures for conjugating daunomycin and adriamycin to antibodies are described by Hurwitz, E. et al. (1975) Cancer Res., 35:1175-1181 and Arnon et al. (1982) Cancer Surv. 1:429-449. Procedures for preparing antibody-ricin conjugates are described in U.S. Pat. No. 4,414,148 and by Osawa, T., et al. (1982) Cancer Surv. 1:373-388 and the references cited therein. Coupling procedures as also described in EP 86309516.2.

In one embodiment, prodrugs are used. For example, to inhibit or kill normal, benign hyperplastic, or cancerous cells, a first protein ligand is conjugated with a prodrug which is activated only when in close proximity with a prodrug activator. The prodrug activator is conjugated with a second protein ligand, preferably one which binds to a non-competing site on the target molecule. Whether two protein ligands bind to competing or non-competing binding sites can be determined by conventional competitive binding assays. Exemplary drug-prodrug pairs suitable for use are described in Blakely et al. (1996) Cancer Res. 56:3287-3292.

Alternatively, the ET1-binding ligand can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985). Other suitable radioisotopes include a-emitters, such as ²¹²Bi, ²¹³Bi, and ²¹¹At, and β-emitters, such as ¹⁸⁶Re and ⁹⁰Y. Moreover, ¹⁷⁷Lu may also be used as both an imaging and cytotoxic agent.

Radioimmunotherapy (RIT) using antibodies labeled with ¹³¹I, , ⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of ⁹⁰Y may be good for bulky tumors. The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of ²⁷⁷Lu-labeled antibodies in the treatment of various cancers. (Mulligan T. et al. (1995) Clin. Cancer Res. 1:1447-1454; Meredith R. F., et al. (1996) J. Nucl. Med. 37:1491-1496; Alvarez R. D., et al. (1997) Gynecol. Oncol. 65:94-101).

The ET1-binding ligands can be used directly in vivo to eliminate antigen-expressing cells via natural complement-dependent cytotoxicity (CDC) or antibody-dependent cellular cytotoxicity (ADCC). The ligands described herein can include a complement binding effector domain, such as the Fc portions from IgG-1, -2, or -3 or corresponding portions of IgM which bind complement. In another embodiment, target cells coated with the ligand which includes a complement binding effector domain are lysed by complement.

Also encompassed by the present invention is a method of killing or inhibiting which involves using the ET1-binding ligand for prophylaxis. For example, these materials can be used to prevent or delay development or progression of cancers.

Use of the disclosed therapeutic methods to treat cancers has a number of benefits. Since the ligands specifically recognize ET1, other tissue is spared and high levels of the agent are delivered directly to the site where therapy is required. Treatment in accordance with the present invention can be effectively monitored with clinical parameters. Alternatively, these parameters can be used to indicate when such treatment should be employed.

ET1 -binding ligands described herein can be administered in combination with one or more of the existing modalities for treating cancers, including, but not limited to: surgery, radiation therapy, and chemotherapy.

It is also possible to deliver an ET1-binding ligand using a gene delivery vehicle. Various methods of transferring or delivering DNA to cells for expression of a therapeutic protein, otherwise referred to as gene therapy, are known. See, for example, Gene Transfer into Mammalian Somatic Cells in vivo, N. Yang (1992) Crit. Rev. Biotechn. 12:335-356. Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies. Gene therapy vectors can be delivered to a subject by, for example, intravenous injection, local administration (see U.S. Pat. 5,328,470) or by stereotactic injection (see e.g., Chen et al. (1994) Proc. Natl. Acad. Sci. USA 91:3054-3057). See also, Methods in Enzymology, Volume 346: Gene Therapy Methods by M. Ian Phillips (Editor), Ian Phillips (Editor) Academic Press February 2002, ISBN: 0121822478.

Diagnostic Uses

Ligands that bind to ET1, e.g., peptide ligands described herein, also have in vitro and in vivo diagnostic utilities.

In one aspect, the present invention provides a diagnostic method for detecting the presence of a ET1, in vitro (e.g., a biological sample, such as tissue, biopsy, e.g., a cancerous tissue) or in vivo (e.g., in vivo imaging in a subject).

The method includes: (i) contacting a sample with an ET1-binding ligand and (ii) detecting formation of a complex between the ET1-binding ligand and the sample. The method can also include contacting a reference sample (e.g., a control sample) with the ligand and determining the extent of formation of the complex between the ligand and the sample relative to the same for the reference sample. A change, e.g., a statistically significant change, in the formation of the complex in the sample or subject relative to the control sample or subject can be indicative of the presence of ET1 in the sample.

Another method includes: (i) administering the ET1-binding ligand to a subject and (iii) detecting formation of a complex between the ET1-binding ligand and the subject. The detecting can include determining location or time of formation of the complex.

The ET1-binding ligand can be directly or indirectly labeled with a detectable substance to facilitate detection of the bound or unbound antibody. Suitable detectable substances include various enzymes, prosthetic groups, fluorescent materials, spin-labels, luminescent materials, and radioactive materials.

Complex formation between the ET1-binding ligand and ET1 can be detected by measuring or visualizing either the ligand bound to the ET1 or unbound ligand. Conventional detection assays can be used, e.g., an enzyme-linked immunosorbent assays (ELISA), a radioimmunoassay (RIA), or tissue immunohistochemistry. As an alternative to labeling the ET1-binding ligand, the presence of ET1 can be assayed in a sample by a competition immunoassay utilizing standards labeled with a detectable substance and an unlabeled ET1-binding ligand. In one example of this assay, the biological sample, the labeled standards, and the ET1-binding agent are combined and the amount of labeled standard bound to the unlabeled ligand is determined. The amount of ET1 in the sample is inversely proportional to the amount of labeled standard bound to the ET1-binding ligand.

Fluorophore and chromophore labeled protein ligands can be prepared. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science, 162:526 and Brand, L. et al. (1972) Annual Review of Biochemistry, 41:843-868. The protein ligands can be labeled with fluorescent chromophore groups by conventional procedures such as those disclosed in U.S. Pat. Nos. 3,940,475; 4,289,747; and 4,376,110. One group of fluorescers having a number of the desirable properties described above is the xanthene dyes, which include the fluoresceins and rhodarnines. Another group of fluorescent compounds are the naphthylamines. Once labeled with a fluorophore or chromophore, the protein ligand can be used to detect the presence or localization of the ET1 in a sample, e.g., using fluorescent microscopy (such as confocal or deconvolution microscopy).

Histological Analysis. Imniunohistochemistry can be performed using the protein ligands described herein. For example, a peptide or Kunitz domain ligand can be synthesized with a label (such as a purification or epitope tag), or can be detectably labeled, e.g., by conjugating a label or label-binding group. The ligand is then contacted to a histological preparation, e.g., a fixed section of tissue that is on a microscope slide. After an incubation for binding, the preparation is washed to remove unbound ligand. The preparation is then analyzed, e.g., using microscopy, to identify if the ligand bound to the preparation.

Of course, the ligand, e.g., the ET1-binding Kunitz domain or peptide, can be unlabeled at the time of binding. After binding and washing, the ligand is labeled in order to render it detectable.

Protein Arrays. The ET1-binding ligand can also be immobilized on a protein array. The protein array can be used as a diagnostic tool, e.g., to screen medical samples (such as isolated cells, blood, sera, biopsies, and the like). Of course, the protein array can also include other ligands, e.g., other ligands that bind to the ET1 and/or ligands that bind to other target molecules, such as ET2, urokinase, or basement membrane components.

Methods of producing polypeptide arrays are described, e.g., in De Wildt et al. (2000) Nat. Biotechnol. 18:989-994; Lueking et al. (1999) Anal. Biochem. 270:103-111; MacBeath and Schreiber (2000) Science 289:1760-1763; WO 01/40803; and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

In one example, cells that produce the protein ligands can be grown on a filter in an arrayed format. Polypeptide production is induced and the expressed polypeptides are immobilized to the filter at the location of the cell.

A protein array can be contacted with a labeled target to determine the extent of binding of the target to an immobilized ET1-binding ligand. If the target is unlabeled, a sandwich method can be used, e.g., using a labeled probe, to detect binding of the unlabeled target.

Information about the extent of binding at each address of the array can be stored as a profile, e.g., in a computer database. The protein array can be produced in replicates and used to compare binding profiles, e.g., of a test sample (e.g., from a patient) and a reference (e.g., recombinant protein or a normal subject). Thus, protein arrays can be used to detect ET1 in a sample.

In vivo Imaging. In still another embodiment, the invention provides a method for detecting the presence of ET1-expressing tissues in vivo. The method includes (i) administering to a subject (e.g., a patient having a cancer or neoplastic disorder) an ET1-binding ligand (e.g., an ET1-binding peptide or an ET1-binding Kunitz domain) physically associated with (e.g., conjugated to or packaged with) a detectable marker; (ii) exposing the subject to a means for detecting said detectable marker on the ET1-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, can also be employed. The protein ligand can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Meth. Enzymol. 121: 802-816.

A radiolabeled ligand of this invention can also be used for in vitro diagnostic tests. The specific activity of an isotopically-labeled ligand depends upon the half-life, the isotopic purity of the radioactive label, and how the label is incorporated into the antibody.

Procedures for labeling polypeptides with the radioactive isotopes (such as ¹⁴C, ³H,³⁵S, ¹²⁵I, ³²P, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology, 2nd ed. London, Orlando: Academic Press( 1986) pp 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,110. Radiolabeling elements which are useful in imaging include ¹²³I, ¹³¹I, ¹¹¹In, and ^{99m}Tc, for example. Procedures for iodinating antibodies are described by Greenwood, F. et al. (1963) Biochem. J. 89:114-123; Marchalonis, J. (1969) Biochem. J. 113:299-305; and Morrison, M. et al. (1971) Immunochemistry 289-297. Procedures for ^{99m}Tc-labeling are described by Rhodes, B. et al. in Burchiel, S. et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich, D. J. et al. (1983) J. Immul. Methods 65:147-157; Hnatowich, D. et al. (1984) J. Applied Radiation 35:554-557; and Buckley, R. G. et al. (1984) F.E.B.S. 166:202-204.

In the case of a radiolabeled ligand, the ligand is administered to the patient, is localized to the tumor bearing the antigen with which the ligand reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subjects, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents, paramagnetic agents (which primarily alter T1), and ferromagnetic or superparamagnetic agents (which primarily alter T2 response). Chelates (e.g., EDTA, DTPA, and NTA chelates) can be used to attach (and reduce toxicity) of some paramagnetic substances (e.g., Fe⁺³, Mn⁺², and Gd⁺³). Other agents can be in the form of particles, e.g., of less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers, such as sepharose, dextran, dextrin, starch and the like.

The ET1-binding ligands can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost; and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image cancerous tissues.

Kits can be prepared that include a ligand that binds to ET1 and instructions for diagnostic use, e.g., the use of the ET1-binding ligand (e.g., an ET1-binding peptide or an ET1-binding Kunitz domain,) to detect ET1, in vitro, e.g., in a sample, e.g., a biopsy or cells from a patient having a cancer or neoplastic disorder, or in vivo, e.g., by imaging a subject. The kit can further contain at least one additional reagent, such as a label or additional diagnostic agent. For in vivo use the ligand can be formulated as a pharmaceutical composition.

Kits

An ET1-binding ligand described herein can be provided in a kit. The kit includes (a) the ET1-binding ligand, e.g., a composition that includes an ET1-binding ligand and, optionally, (b) informational material. The informational material can be descriptive, instructional, marketing or other material that relates to the methods described herein and/or the use of the ET1-binding ligand for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to use of the ET1 -binding ligand to treat an endothelial cell-based disorder, a disorder characterized by undesired angiogenesis, a disorder characterized by insufficient angiogenesis, or a neoplastic disorder.

In one embodiment, the informational material can include instructions to administer the ET1 -binding ligand in a suitable manner to perform the methods described herein, e.g., in a suitable dose, dosage form, or mode of administration (e.g., a dose, dosage form, or mode of administration described herein). Preferred doses, dosage forms, or modes of administration are parenteral, e.g., intravenous, intramuscular, or subcutaneous. In another embodiment, the informational material can include instructions to administer the ET1-binding ligand to a suitable subject, e.g., a human, e.g., a human having or at risk for an endothelial cell-based disorder, a disorder characterized by undesired angiogenesis, a disorder characterized by insufficient angiogenesis, or a neoplastic disorder. For example, the material can include instructions to administer the ET1-binding ligand to a such a subject.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about an ET1-binding ligand and/or its use in the methods described herein. Of course, the informational material can also be provided in any combination of formats.

In addition to an ET1-binding ligand, the composition of the kit can include other ingredients, such as a solvent or buffer, a stabilizer, a preservative, and/or a second agent for treating a condition or disorder described herein, e.g., an endothelial cell-based disorder, a disorder characterized by undesired angiogenesis, a disorder characterized by insufficient angiogenesis, or a neoplastic disorder. Alternatively, other ingredients can be included in the kit, but in different compositions or containers than the ET1-binding ligand. In such embodiments, the kit can include instructions for admixing the ET1-binding ligand and the other ingredients, or for using an ET1-binding ligand together with the other ingredients.

The ET1-binding ligand can be provided in any form, e.g., liquid, dried, or lyophilized form. It is preferred that the ET1-binding ligand be substantially pure and/or sterile. When the ET1-binding ligand is provided in a liquid solution, the liquid solution preferably is an aqueous solution, with a sterile aqueous solution being preferred. When the ET1-binding ligand is provided as a dried form, reconstitution generally is by the addition of a suitable solvent. The solvent, e.g., sterile water or buffer, can optionally be provided in the kit.

The kit can include one or more containers for the composition containing the ET1-binding ligand. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an ET1-binding ligand. For example, the kit includes a plurality of syringes, ampules, foil packets, or blister packs, each containing a single unit dose of an ET1-binding ligand. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is an implantable delivery device.

The following invention is further illustrated by the following examples, which should not be construed as further limiting.

### EXAMPLE 1

### Peptides that Bind to ET1

Two selection strategies were employed to identify peptides that bind to ET1. In the first, a straightforward selection for binders to ET1 was performed to identify binders to multiple epitopes on ET1. Because the substrate-binding cleft of a protease is a natural binding site for a peptide, we predicted that many binders would bind near the active site and inhibit the enzyme by competing with the substrate.

The second selection strategy was a subtractive selection designed to exclusively select for peptides that bound to the enzyme's active site. To this end, the library was first treated with immobilized ET1 that had been covalently inactivated by a small molecule serine protease inhibitor, 4-(2-aminoethyl)benzene sulfonyl fluoride (AEBSF). The AEBSF occluded the active site of the endotheliase, effectively eliminating the epitope. Thus, any library members that bound endotheliase remotely from the active site bound the inactivated enzyme, while those that recognized the active site (and those that do not bind at all) remained in solution. The binders to the inactivated enzyme were discarded and the remaining library members in the solution were treated with immobilized active ET1. Library members that bound to the active site of ET1 were selected for and characterized further.

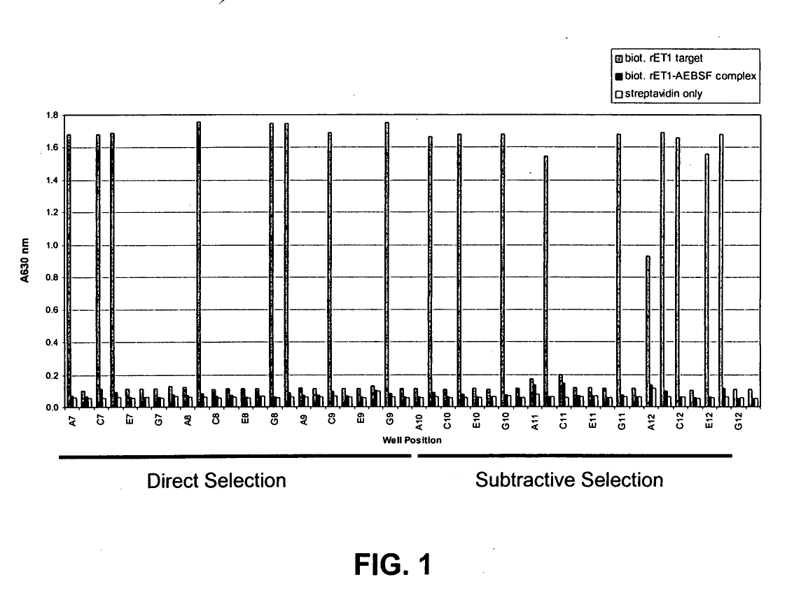

Library members recovered from the selections were tested for ET1 binding by phage ELISA (See FIG. 1). Each isolate was tested for binding to ET1 (first column of each set), covalently inactivated ET1 (second column of each set), and a blank streptavidin well (third column of each set). By examining the binding specificity between the active and inactivated enzyme, it should be possible to determine the isolates that bind to the enzyme's active site. Those isolates that are incapable of binding to the inactivated enzyme may be sterically restricted by the AEBSF, while those that bind remotely from the active site should be unhindered. Indeed, the direct selection yielded isolates that compete with AEBSF, and thus likely target the enzyme's active site. Unexpectedly, only a few isolates bind to both the inhibited and uninhibited enzyme. This result may be due to a particularly good peptide binding cleft in the ET1 active site. The subtractive selection behaved as expected, exclusively producing binders directed toward the enzyme's active site.

ELISA-positive selectants were identified by DNA sequencing, and synthetic peptides were made from the translated sequences. The synthetic peptides were tested for their ability to inhibit ET1 and crude IC₅₀s were measured. To examine the selectivity of the inhibitors, they were tested for their effect on ET2. See Table 1.

TABLE 1

Peptide Inhibitors of ET1

Apparent Kᵢ's

| | | | |
|---|---|---|---|
| SEQ ID | Peptide | ET1IC₅₀ | ET2IC₅₀ |

| | | | | |
|---|---|---|---|---|
| NOs | Name | AA Sequence | (nM) | (nM) |

| | | | | |
|---|---|---|---|---|
| 86 | DX-1054 | AGFQKCKGLYPDCYVPGT | 10000 | NI |
| 87 | DX-1053 | AGMMMCKGLVPECKGGT | 6200 | NI |
| 88 | DX-1052 | AGTAHCFTKDFPCIIFGT | 2800 | NI |
| 89 | DX-1099 | GSHSVCTRDLPISYCVPNAP | 2400 | NI |
| 90 | DX-1102 | AGHWNCKGFAPDCEFIGT | 1400 | NI |
| 91 | DX-1103 | AGHWQCKGFWPDCIPSAT | 950 | NI |
| 92 | DX-1098 | GDRSPCGRWGKTDTKMCQDWDP | 910 | NI |
| 93 | DX-1105 | AGIKHCLSRDTPCITFGT | 830 | NI |
| 94 | DX-1051 | AGVQHCESRDLPCLIKGT | 770 | NI |
| 95 | DX-1097 | GDRGDCEVKMYPWPDKCKHRDPT | 760 | NI |
| 96 | DX-1055 | AGAGKCKGFWPDCYHQGT | 700 | NI |
| 97 | DX-1104 | AGHWQCKGYAPDCEPWGT | 570 | NI |
| 98 | DX-1101 | AGAHTCESRDIPCTVKGTY | 490 | NI |
| 99 | DX-1107 | AGKWHCKGYAPDCQMWGT | 470. | NI |
| 100 | DX-1039 | AGKHICKGYYPDCGYPGT | 450 | — |
| 101 | DX-1129 | AGEYRCKGYWPDCASFGT | 430 | NI |
| 102 | DX-1041 | GSSWYCDKAHPARCWNPAP | 390 | — |
| 103 | DX-1108 | AGMWSCKGYFPDCSNMGT | 360 | NI |
| 104 | DX-1133 | AGNYMCKGYWPDCKMTGT | 350 | NI |
| 105 | DX-1128 | AGEFPCRGFYPDCGYMGT | 300 | NI |
| 106 | DX-1130 | AGHFMCRGYAPDCKPWGT | 210 | NI |
| 107 | DX-1106 | AGKMRCLSRDLPCVTHGTY | 198 | NI |
| 108 | DX-1050 | AGWWPCKGYEPDCPTNGT | 170 | NI |
| 109 | DX-1127 | AGAWLCKGYPPDCAQQGT | 160 | NI |
| 110 | DX-1131 | AGIGMCKGYPPDCIGRGT | 150 | NI |
| 111 | DX-1132 | AGNWYCKGLYPDCMHKGT | 110 | NI |
| 112 | DX-1135 | AGWPTCRGFWPDCGMMGT | 47 | NI |
| 113 | DX-1043 | AQGDNIGVWLWAPYSKGFAWQLGG | NI | — |
| 114 | DX-1045 | AQNREHSSKFGTVRYSTLGPPPGG | NI | — |
| 115 | DX-1046 | AQGMQDEGGAIRHKGAWYWMMAGG | NI | — |
| 116 | DX-1038 | GSQHICHPGGCEKPAP | NI | — |
| 117 | DX-1040 | AGLRKCGFWGFPCKGMGT | NI | — |
| 118 | DX-1042 | GDYLQCRWNAWENRTLCTWRDP | NI | — |
| 119 | DX-1044 | GSNGHCDNHCQMNAP | NI | — |
| 120 | DX-1073 | AGGFKCISEEEDCKLMGT | NI | NI |
| 121 | DX-1074 | AGPDPCRMQGPWCTPMGT | NI | NI |
| 122 | DX-1075 | AGTEFCWLHKGICKTWGT | NI | NI |
| 123 | DX-1076 | AGTMSCDGSMVPCYTPGT | NI | NI |
| 124 | DX-1077 | AQPHWVPNQPVRDRWQSFPKWLGG | NI | NI |
| 125 | DX-1078 | GDDNECEPDADLSEYECVHRDP | NI | NI |
| 126 | DX-1079 | GDNLFCGHSKYAQDHRCRLYDP | NI | NI |
| 127 | DX-1080 | GDSPHCGSHVTVNEKSCMFYDP | NI | NI |
| 128 | DX-1081 | GSNHICPSMGCKFSAP | NI | NI |
| 129 | DX-1082 | GSSFFCVGPECWTSAP | NI | NI |
| 130 | DX-1083 | GSSMFCDAYYCTDHAP | NI | NI |
| 131 | DX-1084 | GSWDSCNELRCIWDAP | NI | NI |
| 132 | DX-1100 | GSVGLCYQNFCKKIAP | NI | NI |
| 133 | DX-1134 | AGHGECMVASHMCIKHGTY | NI | NI |

NI stands for not inhibited; — stands for not tested.

Many of the resulting peptides are endotheliase inhibitors. Some of these are relatively potent, with IC₅₀ values of less than 500 nM. All of the inhibitors tested were exclusively selective toward ET1 and did not inhibit ET2.

Although the peptides were observed to selectively inhibit ET1 with high potency and selectivity, it was possible the peptides were not strictly inhibitors, but poor substratates with low K_{M} values. (i.e., it was possible the peptides were tightly binding to the active site and slowly being hydrolyzed as substrates). To investigate this possibility, we incubated the peptides with a high concentration of ET1 (100 nM rET1 with 50 μM peptide in PBS, 0.1% Tween (200 μL) overnight at room temperature), and subsequently examined the reaction mixture for evidence of hydrolyzed peptide by LC/MS. Hydrolysis was not observed, indicating the peptides are acting as inhibitors and not merely as competitive substrates.

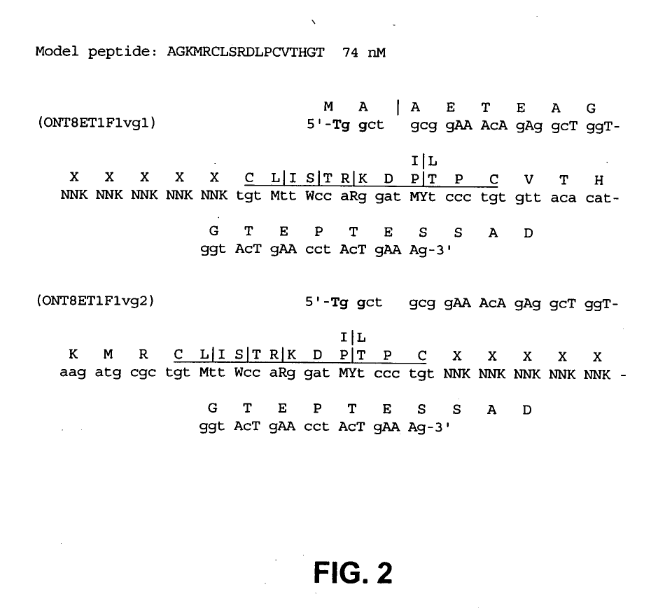

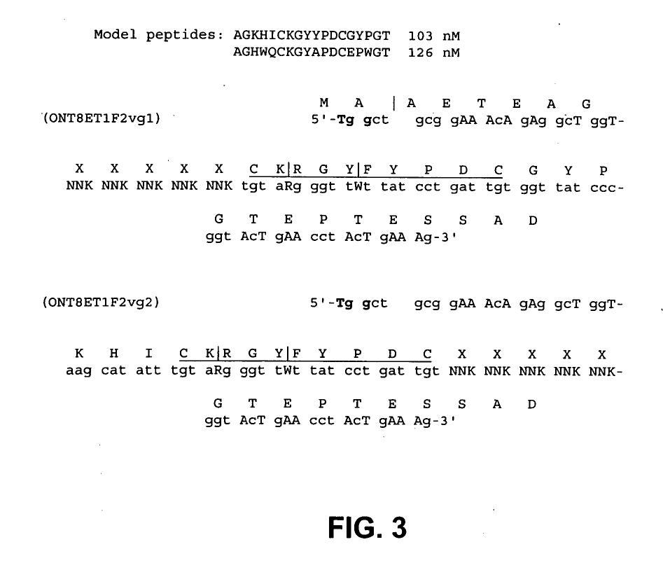

Some of the most potent inhibitors had one of two motifs, either X—X—X—C-(L/I)—(S/T)-(R/K)-D-(I/L/P/T)-P-C—X—X—X (SEQ ID NO:134) (Table 2) or X—X—X—C—(K/R)-G-(Y/F)—Y—P-D-C—X—X—X (SEQ ID NO:135) (Table 3). To further improve the potency of the lead peptide inhibitors, four-second generation libraries were generated, two for each motif. The libraries were prepared such that either amino-terminal or carboxy-terminal flanking sequence was varied but not in combination. The design for the libraries is shown below in FIGS. 2 and 3.

TABLE 2

Summary of Sequences that Define Motifs of the
Most Potent Peptide Inhibitors for Motif 1

| | |
|---|---|
| Motif 1: | |
| X-X-X-C-(L/I)-(S/T)-(R/K)-D-(I/L/P/T)-P-C-X-X-X | (SEQ ID NO:134) |

No. of

| | | |
|---|---|---|
| SEQ ID NO: | Kᵢ | Isolates |

| | | | |
|---|---|---|---|
| 107 | AGKMRCLSRDLPCVTHGTY | 73.6 nM | 4 |
| 93 | AGIKHCLSRDTPCITFGT | 100-1000 nM | 12 |
| 94 | AGVQHCESRDLPCLIKGT | 100-1000 nM | 9 |
| 89 | GSHSVCTRDLPISYCVPNAP | ˜1000 nM | 3 |
| 208 | AGAHTCESRDIPCTVKGT | 100-1000 nM | 1 |
| 199 | GDRRKCISKDTP----CTVHDP | Not Tested | 2 |

TABLE 3

Summary of Sequences that Define Motifs of the Most
Potent Peptide Inhibitors for Motif 2

| | | |
|---|---|---|
| Motif 2: | X-X-X-C-(K/R)-G-(Y/F)-Y-P-D-C-X-X-X | (SEQ ID NO:135) |

| | | |
|---|---|---|
| SEQ ID NO: | Kᵢ | No. of Isolates |

| | | | |
|---|---|---|---|
| 100 | AGKHICKGYYPDCGYPGT | 103 nM | 22 |
| 97 | AGHWQCKGYAPDCEPWGT | 126 nM | 4 |
| 104 | AGNYMCKGYWPDCKMTGT | 256 nM | 1 |
| 99 | AGKWHCKGYAPDCQMWGT | 500 nM | 2 |
| 112 | AGWPTCRGFWPDCGMMGT | 100-1000 nM | 1 |

Since it is likely that a large fraction of the peptides contained in the second generation libraries bind with similar affinities as the parental peptides, we took steps to minimize the recovery of peptides with low affinity. The selection strategy employed for the second generation library involved binding the library to biotinylated target in solution, capture of the target/phage complex on streptavidin beads, extensive washing, and competition elution of low affinity binding phage with the parental peptide. Finally those phage that remained bound to the streptavidin beads were recovered by direct infection of E. coli with the phage coated beads.

Output phage from the selections were analyzed by ELISA and then ELISA positives were sequenced. Amino acid sequences of twelve peptides, based on the two motifs, are shown in Table 4 and Table 5 below. These sequences may include at least two or three amino- and two or three carboxy-terminal amino acids which are optional.

TABLE 4

Summary of Peptide Sequences Obtained from
a Second Generation

Library

Selection

| | |
|---|---|
| SEQ ID NO:136 | AGQMRRKCISRDIPCVTHGTY |
| SEQ ID NO:137 | AGQVRRYCISRDIPCVTHGT |
| SEQ ID NO:138 | AGRSRVRCISRDIPCVTHGTY |
| SEQ ID NO:139 | AGSGRRFCISRDIPCVTHGT |
| SEQ ID NO:140 | AGMARVKCISRDIPCVTHGTY |
| SEQ ID NO:141 | AGKMRCISRDIPCTVKSGGTY |
| SEQ ID NO:142 | AGKMRCLSRDIPCSIHQKGTY |
| SEQ ID NO:143 | AGKMRCLSRDIPCVNFRLGT |
| SEQ ID NO:144 | AGKMRCISRDIPCTVFQEGT |
| SEQ ID NO:145 | AGKMRCISRDIPCTTRVAGTY |
| SEQ ID NO:146 | AGKMRCISRDIPCSHYQIGT |

TABLE 5

Summary of Peptide
Sequences Obtained from a Second Generation

Library Selection

| | |
|---|---|
| SEQ ID NO:147 | AGGWRYPCKGFYPDCGYPGT |
| SEQ ID NO:148 | AGNTGWRCKGYYPDCGYPGT |
| SEQ ID NO:149 | AGRASWRCKGYYPDCGYPGT |
| SEQ ID NO:150 | AGRETWVCKGYYPDCGYPGT |
| SEQ ID NO:151 | AGRAGWRCKGYYPDCGYPGT |
| SEQ ID NO:152 | AGQLGWKCKGYYPDCGYPGT |
| SEQ ID NO:153 | AGSSGWRCKGYYPDCGYPGT |
| SEQ ID NO:154 | AGKHICRGFYPDCVWQTWGT |
| SEQ ID NO:155 | AGKHICRGYYPDCVWQTWGT |
| SEQ ID NO:156 | AGKHICRGYYPDCVWQTFGT |
| SEQ ID NO:157 | AGKHICRGYYPDCIWQFAGT |
| SEQ ID NO:158 | AGKHICRGFYPDCVWQTFGT |
| SEQ ID NO:159 | AGKHICRGYYPDCEWQIFGT |

Apparent Kᵢ's were measured for inhibition of ET1 for the peptides shown in Table 6. These are uncorrected for substrate binding to the enzyme. It is expected that any correction would be small if the peptides are competitive inhibitors and there is no correction if the peptides are non-competitive inhibitors.

Any of the peptides described herein can be modified by attachment of a chemical moiety that prolongs residence in the circulation system (see also discussion above). In one embodiment, the peptides are attached to PEG. PEGs of various lengths could be used. For example, the PEG could be 5,000 Da, 8,000 Da, 10,000 Da, 20,000 Da, or 30,000 Da. Such PEGylated peptides could be administered (e.g., injected) into a patient in need of ET1 inhibition or for ET1 detection and would remain in the blood stream with a half-life of, for example, 1 hour, 2 hours, 3 hours, 4 hours, 8 hours, 1 day, 2 days, 14 or more days.

Other moieties can also be used to prolong serum residence. For example, a moiety that causes an association (e.g., a covalent or non-covalent association) between an ET1-binding ligand and a serum protein can be used. For example, the moiety can be a crosslinker such as maleimide which causes a covalent attachment of serum albumin (SA) and other serum proteins. In another embodiment, the moiety includes fatty acids and other hydrophobic organic groups and mediates non-covalent binding to SA.

Moieties that prolong serum residence of peptides and small proteins can be attached by standard chemistry. For peptides, the serum-residence prolonging moiety can be attached in a way that does not interfere with the activity of the peptide. Since many peptides described herein were selected from a display library, they clearly function when attached to large moiety. Phage are approximately 20 million Daltons in molecular weight, and the peptides were attached to the phage by their carboxy terminus. Thus, attaching PEG or other moieties to these peptides, e.g., at their carboxy terminus, is unlikely to affect the binding and inhibitory activity of the peptide. In one embodiment, the peptide is extended by a few residues such as GGGK. Serum-residence prolonging moieties can also be attached to the amino terminal residue. The peptide can also be extended by one to ten residues from the amino terminus to separate the serum-residence prolonging moiety from the binding site of the peptide.

For Kunitz domains, serum-residence prolonging moieties can be attached to the amino-carboxy terminus or to one or more of the lysines of the Kunitz domain.

TABLE 6
Kᵢ^{apparent} against ET1

| | | | |
|---|---|---|---|
| | | | Kᵢ^{App} |
| SEQ ID NOs | DX# | Sequence | (nM) |

| | | | |
|---|---|---|---|
| 160 | 1626 | AGQVRRYCISRDIPCVTHGT | 16.6 |
| 161 | 1628 | AGQMRRKCISRDIPCVTHGTY | 10.2 |
| 162 | 1629 | AGRSRVRCISRDIPCVTHGTY | 144.6 |
| 163 | 1630 | AGSGRRFCISRDIPCVTHGT | 128.2 |
| 164 | 1631 | AGMARVKCISRDIPCVTHGTY | 13.1 |
| 165 | 1632 | AGKMRCISRDIPCTVKSGGTY | 15.8 |
| 166 | 1633 | AGKMRCLSRDIPCSIHQKGTY | 26.4 |
| 167 | 1634 | AGKMRCLSRDIPCVNFRLGT | 19.3 |
| 168 | 1635 | AGKMRCISRDIPCTVFQEGT | 8.5 |
| 169 | 1636 | AGKMRCISRDIPCTTRVAGTY | 11.3 |
| 170 | 1637 | AGKMRCISRDIPCSHYQIGT | 8.5 |
| 171 | 1638 | AGGWRYPCKGFYPDCGYPGT | 126.5 |
| 172 | 1639 | AGNTGWRCKGYYPDCGYPGT | 28 |
| 173 | 1640 | AGRASWRCKGYYPDCGYPGT | 29 |
| 174 | 1641 | AGRETWVCKGYYPDCGYPGT | 34.6 |
| 175 | 1642 | AGRAGWRCKGYYPDCGYPGT | 11.6 |
| 176 | 1643 | AGQLGWKCKGYYPDCGYPGT | 25.6 |
| 177 | 1644 | AGSSGWRCKGYYPDCGYPGT | 8.6 |
| 178 | 1645 | AGKHICRGFYPDCVWQTWGT | 15.6 |
| 179 | 1646 | AGKHICRGYYPDCVWQTWGT | 8.6 |
| 180 | 1647 | AGKHICRGYYPDCVWQTFGT | 12.7 |
| 181 | 1648 | AGKHICRGYYPDCIWQFAGT | 19.8 |
| 182 | 1649 | AGKHICRGFYPDCVWQTFGT | 22.1 |
| 183 | 1650 | AGKHICRGYYPDCEWQIFGT | 33.6 |

### EXAMPLE 2

### Kunitz Domains that Bind to ET1

A monovalent Kunitz library has been used to identify inhibitors to recombinant endotheliase 1 (rET1). Three rounds of selection were performed. Phage were incubated with the biotinylated rET1 target in solution for two hours. After binding, the phage-target complexes were captured on streptavidin coated magnetic beads. ELISA analysis of phage isolates from the third round was performed using rET1 coated plates and an anti-geneVIII antibody to detect the phage. To examine the specificity of the selected Kunitz domains, the phage isolates were tested for their ability to recognize ET2 in the ELISA assay. Results from this ELISA are shown in Table 7 and Table 8.

TABLE 7
Phage ELISA Data (1)

| Phage | | | |
|---|---|---|---|
| isolate | rET-1 | rET-2 | Strep. |
| A1 | 0.291 | 0.051 | 0.050 |
| B1 | 0.238 | 0.052 | 0.057 |
| C1 | 0.232 | 0.086 | 0.057 |
| D1 | 0.056 | 0.050 | 0.050 |
| E1 | 0.368 | 0.060 | 0.058 |
| F1 | 0.491 | 0.153 | 0.052 |
| G1 | 0.083 | 0.053 | 0.049 |
| H1 | 0.346 | 0.120 | 0.048 |
| A2 | 0.493 | 0.092 | 0.051 |
| B2 | 0.509 | 0.054 | 0.053 |
| C2 | 0.061 | 0.052 | 0.052 |
| D2 | 0.056 | 0.055 | 0.053 |
| E2 | 0.490 | 0.200 | 0.054 |
| F2 | 0.096 | 0.054 | 0.052 |
| G2 | 0.429 | 0.056 | 0.054 |
| H2 | 0.160 | 0.053 | 0.051 |
| A3 | 0.093 | 0.050 | 0.052 |
| B3 | 0.313 | 0.052 | 0.049 |
| C3 | 0.055 | 0.059 | 0.052 |
| D3 | 0.463 | 0.154 | 0.053 |
| E3 | 0.460 | 0.169 | 0.052 |
| F3 | 0.173 | 0.060 | 0.050 |
| G3 | 0.054 | 0.051 | 0.052 |
| H3 | 0.211 | 0.055 | 0.051 |
| A4 | 0.498 | 0.103 | 0.046 |
| B4 | 0.401 | 0.109 | 0.048 |
| C4 | 0.308 | 0.050 | 0.049 |
| D4 | 0.305 | 0.047 | 0.047 |
| E4 | 0.359 | 0.191 | 0.048 |
| F4 | 0.382 | 0.070 | 0.048 |
| G4 | 0.259 | 0.080 | 0.048 |
| H4 | 0.224 | 0.047 | 0.046 |
| A5 | 0.154 | 0.052 | 0.051 |
| B5 | 0.400 | 0.155 | 0.050 |
| C5 | 0.257 | 0.050 | 0.053 |
| D5 | 0.057 | 0.053 | 0.054 |
| E5 | 0.191 | 0.091 | 0.052 |
| F5 | 0.504 | 0.135 | 0.052 |
| G5 | 0.299 | 0.057 | 0.053 |
| H5 | 0.388 | 0.053 | 0.052 |
| A6 | 0.054 | 0.050 | 0.051 |
| B6 | 0.167 | 0.050 | 0.049 |
| C6 | 0.570 | 0.231 | 0.052 |
| D6 | 0.055 | 0.052 | 0.050 |
| E6 | 0.399 | 0.057 | 0.053 |
| F6 | 0.254 | 0.058 | 0.056 |
| G6 | 0.129 | 0.055 | 0.055 |
| H6 | 0.268 | 0.108 | 0.052 |
| (+) Con | 0.543 | 0.061 | 0.059 |

TABLE 8
Phage ELISA Data (2)

| Phage | | | |
|---|---|---|---|
| isolate | rET-1 | rET-2 | Strep. |
| A7 | 0.054 | 0.053 | 0.050 |
| B7 | 0.280 | 0.049 | 0.048 |
| C7 | 0.399 | 0.111 | 0.052 |
| D7 | 0.419 | 0.178 | 0.050 |
| E7 | 0.055 | 0.053 | 0.053 |
| F7 | 0.495 | 0.102 | 0.051 |
| G7 | 0.386 | 0.052 | 0.051 |
| H7 | 0.463 | 0.194 | 0.053 |
| A8 | 0.308 | 0.060 | 0.052 |
| B8 | 0.082 | 0.048 | 0.048 |
| C8 | 0.289 | 0.051 | 0.051 |
| D8 | 0.271 | 0.052 | 0.053 |
| E8 | 0.538 | 0.230 | 0.053 |
| F8 | 0.482 | 0.057 | 0.053 |
| G8 | 0.543 | 0.057 | 0.053 |
| H8 | 0.396 | 0.053 | 0.052 |
| A9 | 0.285 | 0.082 | 0.048 |
| B9 | 0.320 | 0.054 | 0.048 |
| C9 | 0.126 | 0.052 | 0.046 |
| D9 | 0.410 | 0.051 | 0.051 |
| E9 | 0.535 | 0.055 | 0.053 |
| F9 | 0.351 | 0.092 | 0.051 |
| G9 | 0.404 | 0.051 | 0.049 |
| H9 | 0.339 | 0.059 | 0.052 |
| A10 | 0.455 | 0.048 | 0.050 |
| B10 | 0.118 | 0.055 | 0.050 |
| C10 | 0.339 | 0.059 | 0.055 |
| D10 | 0.380 | 0.097 | 0.049 |
| E10 | 0.088 | 0.049 | 0.050 |
| F10 | 0.441 | 0.152 | 0.049 |
| G10 | 0.354 | 0.053 | 0.051 |
| H10 | 0.363 | 0.051 | 0.050 |
| A11 | 0.055 | 0.051 | 0.059 |
| B11 | 0.441 | 0.085 | 0.051 |
| C11 | 0.123 | 0.051 | 0.049 |
| D11 | 0.314 | 0.062 | 0.051 |
| E11 | 0.382 | 0.137 | 0.052 |
| F11 | 0.300 | 0.070 | 0.051 |
| G11 | 0.498 | 0.059 | 0.052 |
| H11 | 0.063 | 0.062 | 0.056 |
| A12 | 0.141 | 0.053 | 0.052 |
| B12 | 0.146 | 0.054 | 0.048 |
| C12 | 0.271 | 0.083 | 0.049 |
| D12 | 0.402 | 0.046 | 0.049 |
| E12 | 0.101 | 0.049 | 0.052 |
| F12 | 0.473 | 0.126 | 0.052 |
| G12 | 0.154 | 0.052 | 0.050 |
| (+) Con | 0.543 | 0.061 | 0.059 |

ELISA analysis indicates that there 79/95 isolates have a signal >2 times background (streptavidin only). 29/95 isolates show some reactivity towards rET2. 27/95 isolates have a signal >8 times background. 12/95 isolates have a signal >8 times background with no reactivity towards rET2.

Sequence analysis was performed on the 12 isolates that gave the strongest ELISA signal. The sequencing results are shown in Table 9. All 12 isolates appear to be unique. These exemplary sequences may include at least four or five amino- and four or six or ten carboxy-terminal amino acids which are optional.

TABLE 9
Exemplary ET1-binding Kunitz Domains

| SEQ | | |
|---|---|---|
| ID | | |
| NOs | Identifier | AA sequence appended to MBP |
| 184 | >371A 091802- | MAAEMHSFCAFKADRGPCRADFHRFFFNIFTRQC |
| | a10 (#1) | EEFHYGGCGGNQNRFESLEECKKMCTRDSASSAS |
| | | GDFD |
| 185 | >371A-091802- | MAAEMHSFCAFKADKGFCRAMDIRFFFNIFTRQC |
| | b02 (#2) | EEFIYGGCGGNQNRFESLEECKKMCTRDSASSAS |
| | | GDFD |
| 186 | >371A-091802- | MAAEMHSFCAFKADQGPCRAAISRFFFNIFTRQC |
| | d09 (#3) | EEFVYGGCEGNQNRFESLEECKKMCTRDSASSAS |
| | | GDFD |
| 187 | >371A-091802- | MAAEMHSFCAFKADKGECRASVQRFFFNIFTRQC |
| | d12 (#4) | EEFNYGGCGGNQNRFESLEECKKMCTRDSASSAS |
| | | GDFD |
| 188 | >371A-091802- | MAAEMHSFCAFKADPGPCRAMFNRFFFNIFTRQC |
| | e06 (#5) | EEFNYGGCSGNQNRFESLEECKKMCTRDSASSAS |
| | | GDFD |
| 189 | >371A-091802- | MAAEMHSFCAFKADKGTCRGDFPRFFFNIFTRQC |
| | e09 (#6) | EEFHYGGCGGNQNRFESLEECKKMCTRDSASSAS |
| | | GDFD |
| 190 | >371A-091802- | MAAEMHSFCAFKADQGPCRASVHRFFFNIFTRQC |
| | f8 (#7) | EEFFYGGCLGNQNRFESLEECKKMCTRDSASSAS |
| | | GDFD |
| 191 | >371A-091802- | MAAEMHSFCAFKADPGQCRAYYRRFFFNIFTRQC |
| | g02 (#8) | EEFVYGGCMGNQNRFESLEECKKMCTRDSASSAS |
| | | GDFD |
| 192 | >371A-091802- | MAAEMHSFCAFKADRGPCRAYFDRFFFNIFTRQG |
| | g08 (#9) | EEFIYGGCMGNQNRFESLEECKKMCTRDSASSAS |
| | | GDFD |
| 193 | >371A-091802- | MAAEMHSFCAFKADTGPCRADIKRFFFNIFTRQC |
| | g09 (#10) | EEFRYGGCMGNQNRFESLEECKKMCTRDSASSAS |
| | | GDFD |
| 194 | >371A-091802- | MAAEMHSFCAFKADPGPCRAIMTRFFFNIFTRQC |
| | g11 (#11) | EEFRYGGCLGNQNRFESLEECKKMCTRDSASSAS |
| | | GDFD |
| 195 | >371A-091802- | MAAEMHSFCAFKADTGTCRAAMVRFFFNIFTRQC |
| | h08 (#12) | EEFTYGGCEGNQNRFESLEECKKMCTRDSASSAS |
| | | GDFD |

In order to provide sufficient quantities of material for in vitro inhibition analysis, the twelve isolates that gave the strongest ELISA signal were cloned into an expression vector, pANIX01, which allowed their production as C-terminal fusions to the maltose binding protein (MBP). Cloning into this vector placed a His-tag at the C-terminus of the protein that was used to facilitate purification.

For protein production, E. coli containing the expression vector were grown in media containing ampicillin and 2% glucose to an OD₆₀₀ of 0.5. At this time the cells were spun down and the media removed. The cell pellet was then resuspended in fresh media containing ampicillin and 1 mM IPTG to induce expression of the protein. The cells were allowed to grow with shaking overnight at 30° C. before harvesting by centrifugation. Periplasmic extracts were then prepared and the MBP-Kunitz fusion purified using immobilized metal ion chromatography. Data from in vitro inhibition analysis are summarized in Table 10.

TABLE 10
In Vitro Inhibition

| | T⁰ | T³⁰ | | | | | | |
|---|---|---|---|---|---|---|---|---|
| Kunitz | IC₅₀ | IC₅₀ | MTSP-1 | ET-2 | TnIV | Trypsin | Plasmin | fXa |
| domains | (nM) | (nM) | (nM) | (nM) | (nM) | (nM) | (nM) | (nM) |
| MBP- | 88 | 3.1 | ≧1000 | IA | ˜80 | <10 | ˜100 | IA |
| 1A10 | | | | | | | | |
| MBP-1B2 | 21 | 1.3 | IA | IA | ≧1000 | <10 | ˜500 | IA |
| MBP-1D9 | 164 | 7.1 | IA | ≧1000 | ≧1000 | ˜50 | ˜60 | IA |
| MBP-D12 | 54.3 | 1.2 | IA | IA | ≧1000 | ˜30 | ˜500 | ≧1000 |
| MBP-1E6 | | 4.9 | ˜500 | ˜400 | ˜100 | <10 | ˜30 | IA |
| MBP-1E9 | 66 | 2.1 | IA | IA | ≧1000 | ≧1000 | ≧1000 | IA |
| MBP-1F8 | >400 | 21.4 | IA | IA | IA | <10 | ˜60 | IA |
| MBP-1G2 | 310 | 8.2 | IA | ˜300 | ˜600 | <10 | ˜40 | ˜100 |
| MBP-1G8 | 109 | 2.1 | IA | IA | ˜10 | ˜10 | ˜80 | ≧1000 |
| MBP-1G9 | 646 | 20.7 | ≧1000 | IA | IA | ˜60 | ˜100 | IA |
| MBP- | 120 | 2.7 | IA | ≧1000 | ≧1000 | <10 | ˜50 | ≧1000 |
| 1G11 | | | | | | | | |
| MBP-1H8 | >400 | 15 | IA | IA | IA | ˜30 | ˜60 | IA |

Columns 2 and 3 indicate IC50's for ET1 interaction following a 0 minute (T0, column 2) or a 30 minute (T30, column 3) preincubation.
Six columns on the right indicate estimated IC₅₀'s following a 30 minute preincubation with 100 nM of the respective Kunitz domain.
IA = <5% inhibition @ 100 nM; >1000 = 5-10% inhibition @ 100 nM.
All the Kunitz domains exhibited <5% inhibition (IA) of uPA; all exhibited <5% inhibition (IA) of MTSP-9, except G8, G9, and A10, which exhibited 5-10% inhibition (>1000); all exhibited <5% inhibition (IA) of fIIa, except D12, E9, and A10, which exhibited 5-10% inhibition (>1000).

A number of embodiments of the invention have been described. Nevertheless, it will be understood that various modifications may be made without departing from the spirit and scope of the invention. Accordingly, other embodiments are within the scope of the following claims and the Summary (above).

1. An isolated compound comprising a peptide that binds endotheliase 1 (ET1) with a K_{d} of less than 500 nM wherein the peptide comprises two cysteines that form a disulfide bond and contains fewer than 20 amino acids.

2. The compound of claim 1 wherein the ET1 is human ET1.

3. The compound of claim 1 wherein the peptide binds the active site of ET1.

4. The compound of claim 1 wherein at least one amino acid in the peptide is within 10 Angstroms of the active site serine of ET1 when the compound is bound to ET1.

5. The compound of claim 3 wherein the compound inhibits activity of ET1 with an IC₅₀ of less than 500 nM.

6. The compound of claim 4 wherein the peptide is not cleaved by ET1.

7. The compound of claim 3 wherein the peptide binds to ET1 at least 50-fold more tightly than the peptide binds to ET1 that has been reacted with 4-(2-aminoethyl)benzene sulfonyl fluoride (AEBSF).

8. The compound of claim 4 wherein the peptide does not inhibit trypsinogen-IV, membrane-type serine proteases-1, -6, -7, urokinase-like plasminogen activator (uPA), trypsin, factor IIa, plasmin (Plm), and/or factor Xa or ET2.

9. The compound of claim 1 wherein the peptide comprises two cysteine residues that form a disulfide bond.

10. The compound of claim 9 wherein the first cysteine is separated from the second cysteine by between 4 to 12 amino acids.

11. The compound of claim 1 wherein the compound inhibits angiogenesis.

12. The compound of claim 1 wherein the compound inhibits proteolysis of vessel basement membrane.

13. The compound of claim 1 further comprising a moiety that prolongs serum residence time.

14. The compound of claim 1 wherein the peptide independently binds endotheliase 1 (ET1) with a K_{d} of less than 100 nM, and the peptide comprises the amino acid sequence:

| | |
|---|---|
| X1-X2-X3-C4-X5-X6-X7-X8-X9-X10- | (SEQ ID NO:212) |
| C11-X12-X13-X14, | |

wherein X is any non-cysteine amino acid wherein X1, X2, and X3 can be absent, one or both of X5 and X6 can be absent, X12, X13 and X14 can be absent, and C4 and C11 can form a disulfide bond.

15. The compound of claim 14 comprising, between C4 and C11, an amino acid sequence selected from the group consisting of:

| | |
|---|---|
| KGFAPD, | (SEQ ID NO:55) |
| KGFWPD, | (SEQ ID NO:56) |
| KGLYPD, | (SEQ ID NO:57) |
| KGLVPE, | (SEQ ID NO:58) |
| KGYAPD, | (SEQ ID NO:59) |
| KGYYPD, | (SEQ ID NO:60) |
| KGYWPD, | (SEQ ID NO:61) |
| KGYFPD, | (SEQ ID NO:62) |
| KGYEPD, | (SEQ ID NO:63) |
| KDYPPD, | (SEQ ID NO:64) |
| KGLYPD, | (SEQ ID NO:65) |
| RGFYPD, | (SEQ ID NO:66) |
| RGFWPD, | (SEQ ID NO:67) |
| and | |
| RGYAPD, | (SEQ ID NO:68) |

or an amino acid selected from an amino acid that differs by no more than one amino acid substitution, insertion or deletion from a sequence in the above group.

16. The compound of claim 1 wherein the compound is produced in a cell.

17. The compound of claim 1 wherein the compound is produced by synthetic chemistry.

18. The compound of claim 1 wherein the peptide comprises an amino acid sequence differs by fewer than three amino acid substitutions, insertions, or deletion from an amino acid sequence selected from the group consisting of:

| | |
|---|---|
| RRKCISRDIPCVTH, RRYCISRDIPCVTH, RVRCISRDIPCVTH, | (SEQ ID NOs 9-31 respectively) |
| RRFCISRDIPCVTH, RVKCISRDIPCVTH, KMRCISRDIPCTVK, | |
| KMRCLSRDIPCSIH, KMRCLSRDIPCVNF, KMRCISRDIPCTVF, | |
| KMRCISRDIPCTTR, KMRCISRDLPCSHY, RYPCKGFYPDCGYP, | |
| GWRCKGYYPDCGYP, SWRCKGYYPDCGYP, TWVCKGYYPDCGYP, | |
| GWRCKGYYPDCGYP, GWKCKGYYPDCGYP, GWRCKGYYPDCGYP, | |
| KHICRGFYPDCVWQ, KHICRGYYPDCVWQ, KHICRGYYPDCIWQ, | |
| KHICRGFYPDCVWQ, and KHICRGYYPDCEWQ. | |

19. The compound of claim 18 wherein the compound comprises the sequence KMRCLSRDIPCVNF (SEQ ID NO:16).

20. The compound of claim 1 wherein the peptide comprises an amino acid sequence that differs by fewer than three amino acid substitutions, insertions, or deletion from an amino acid sequence selected from the group consisting of:

| | |
|---|---|
| QMRRKCISRDIPCVTH, QVRRYCISRDIPCVTH, RSRVRCISRDIPCVTH, | (SEQ ID NOs 32-54 respectively) |
| SGRRFCISRDIPCVTH, MARVKCISRDIPCVTH, AGKMRCISRDIPCTVK, | |
| AGKMRCLSRDIPCSIH, AGKMRCLSRDIPCVNF, AGKMRCISRDIPCTVF, | |
| AGKMRCISRDIPCTTR, AGKMRCISRDIPCSHY, GWRYPCKGFYPDCGYP, | |
| NTGWRCKGYYPDCGYP, RASWRCKGYYPDCGYP, RETWVCKGYYPDCGYP, | |
| RAGWRCKGYYPDCGYP, QLGWKCKGYYPDCGYP, SSGWRCKGYYPDCGYP, | |
| AGKHICRGFYPDCVWQ, AGKHICRGYYPDCVWQ, AGKHICRGYYPDCIWQ | |
| AGKHICRGFYPDCVWQ, and AGKHICRGYYPDCEWQ. | |

21. The compound of claim 20 wherein the peptide comprises the sequence SGRRFCISRDlPCVTH (SEQ ID NO:35).

22. The compound of claim 20 wherein the peptide comprises the sequence AGKMRCISRDIPCTVK (SEQ ID NO:37).

23. The compound of claim 20 wherein the peptide comprises the sequence NTGWRCKGYYPDCGYP (SEQ ID NO:44).

24. The compound of claim 20 wherein the peptide comprises the sequence RETWVCKGYYPDCGYP (SEQ ID NO:46).

25. A nucleic acid comprising a sequence encoding a polypeptide that comprises the peptide component of a compound according to claim 1.

26. A compound according to claim 1 further comprising a detectable label.

27. A compound according to claim 1 further comprising a cytotoxin.

28. A compound according to claim 1 further comprising a carrier molecule.

29. An isolated protein comprising a Kunitz domain that binds endotheliase 1 (ET1) with a K_{d} of less than 500 nM, the Kunitz domain comprising the amino acid sequence:

| | |
|---|---|
| X1-X2-X3-X4-C5-X6-X7-X8-X9-X9a-X10-X11-X12-X13-C14-X15-X16-X17- | (SEQ ID NO:5) |
| X18-X19-X20-X21-X22-X23-X24-X25-X26-X27-X28-X29-X29a-X29b-X29c-C30- | |
| X31-X32-X33-X34-X35-X36-X37-C38-X39-X40-X41-X42-X42a-X42b-X43-X44-X45- | |
| X46-X47-X48-X49-X50-C51-X52-X53-X54-C55-X56-X57-X58, | |

wherein X is any amino acid other than cysteine.

30. The protein of claim 29 wherein the Kunitz domain independently binds endotheliase 1 (ET1 ) with a K_{d} of less than 100 nM.

31. The protein of claim 30 wherein the Kunitz domain comprising an amino acid sequence that differs by no more than four amino acid substitutions, insertions, or deletions from an amino acid sequence selected from the group:

| | |
|---|---|
| SFCAFKADRGPCRADFHRFFFNIFTRQCEEFH | (SEQ ID NO:74) |
| YGGCGGNQNRYESLEECKKMCTRDS; | |
| SFCAFKADKGFCRAMDIRFFFNIFTRQCEEFI | (SEQ ID NO:75) |
| YGGCGGNQNRFESLEECKKMCTRDS; | |
| SFCAFKADQGPCRAAISRFFFNIFTRQCEEFV | (SEQ ID NO:76) |
| YGGCEGNQNRFESLEECKKMCTRDS; | |
| SFCAFKADKGECRASVQRFFFNIFTRQCEEFN | (SEQ ID NO:77) |
| YGGCGGNQNRFESLEECKKMCTRDS; | |
| SFCAFKADPGPCRAMFNRFFFNIFTRQCEEFN | (SEQ ID NO:78) |
| YGGCSGNQNRFESLEECKKMCTRDS; | |
| SFCAFKADKGTCRGDFPRFFFNIFTRQCEEFH | (SEQ ID NO79:) |
| YGGCGGNQNRFESLEECKKMCTRDS; | |
| SFCAFKADQGPCRASVHRFFFNIFTRQCEEFF | (SEQ ID NO:80) |
| YGGCLGNQNRFESLEECKKMCTRDS; | |
| SFCAFKADPGQCRAYYRRFFFNIFTRQCEEFV | (SEQ ID NO:81) |
| YGGCMGNQNRFESLEECKKMCTRDS; | |
| SFCAFKADRGPCRAYFDRFFFNIFTRQCEEFI | (SEQ ID NO:82) |
| YGGCMGNQNRFESLEECKKMCTRDS; | |
| SFCAFKADTGPCRADIKRFFFNIFTRQCEEFR | (SEQ ID NO:83) |
| YGGCMGNQNRFESLEECKKMCTRDS; | |
| SFCAFKADPGPCRAIMTRFFFNIFTRQCEEFR | (SEQ ID NO:84) |
| YGGCLGNQNRFESLEECKKMCTRDS; | |
| and | |
| SFCAFKADTGTCRAAMVRFFFNIFTRQCEEFT | (SEQ ID NO:85) |
| YGGCEGNQNRFESLEECKKMCTRDS. | |

32. The protein of claim 31, wherein the Kunitz domain comprises the sequence: SFCAFKADRGPCRAYFDRFFFNIFTRQCEEFIYGGCMGNQNRFESLEECKKMCTR DS (SEQ ID NO:82).

33. A method of modulating ET1 activity in a subject, the method comprising:
administering a ligand that binds to ET1 to the subject in an amount effective to modulate ET1 activity in the subject.

34. The method of claim 33 wherein the ligand is an antagonist of ET1 and the amount is effective to antagonize ET1 activity in the subject.

35. The method of claim 33 wherein the subject has or is at risk for having a neoplasia.

36. The method of claim 33 wherein the subject has or is at risk for having a metastatic cancer.

37. The method of claim 33 wherein the subject has or is at risk for having a disorder characterized by excess angiogenesis.

38. The method of claim 37 wherein the disorder is a disorder selected from the group consisting of: rheumatoid arthritis, psoriasis, diabetic retinopathies, ocular disorder such as pterygii recurrence, surgery (e.g., scarring excimer laser surgery and glaucoma filtering surgery), a cardiovascular disorder, a chronic inflammatory disorder, a circulatory disorder, crest syndrome, and a dermatological disorder.

39. The method of claim 33 wherein the ligand comprises (i) a peptide that comprises two cysteines that can form a disulfide bond and the peptide can bind to ET1 or (ii) a Kunitz domain.

40. A method of reducing angiogenesis in a subject having or at risk for a neoplastic disorder, the method comprising: administering a ligand that binds to ET1 to a subject having or at risk for a neoplastic disorder in an amount effective to reduce angiogenesis in the subject, thereby reducing the ability of a tumor to grow in the subject.

41. The method of claim 40 wherein the subject has or is at risk for having a metastatic cancer.

42. A method of modulating the activity of an ET1-expressing cell, the method comprising: contacting an ET1-expressing cell with a ligand that binds to ET1, thereby modulating the activity of the ET1-expressing cell.

43. A method of modulating proteolysis of a biological structure, the method comprising: contacting the biological structure with a ligand that binds to ET1 in an amount sufficient to modulate proteolysis of the biological structure.

44. A method of detecting endotheliase activity in a sample, the method comprising: contacting the sample with a ligand that binds to ET1, and evaluating interaction between the ligand and a component in the sample.

45. The method of claim 44 wherein the ligand comprises a label; and evaluating the interaction comprises detecting the label.

46. A method of detecting ET1 protein in a subject, the method comprising: administering a ligand that binds to ET1 to the subject, and evaluating the protein in the subject or in a sample from the subject.

47. The method of claim 46 wherein the ligand comprises a label; and evaluating comprises detecting localization of the ligand in the subject or in a sample from the subject.

48. A biopolymer library comprising a plurality of varied biopolymers, wherein each biopolymer of the plurality is a nucleic acid that encodes a protein, or is a protein comprising:

| | |
|---|---|
| C4-X5-X6-X7-X8-X9-X10-C11, | (SEQ ID NO:213) |

(i) wherein X5 is L or , X6 is S or T, X7 is R or K, X8 is D, X9 is I, L, P, or T, and X10 is P, and one or more of positions X5, X6, X7, X8, X9, and X10 are varied,
at least 10² unique proteins are represented by the different biopolymers of the plurality, or
(ii) wherein X5 is K or R, X6 is G, X7 is Y or F, X8 is Y, W, or A, X9 is P, and X10 is D, and one or more of positions X5, X6, X7, X8, X9, and X10 are varied, and at least 10² unique proteins are encoded by the different biopolymers of the plurality.

49. A method of identifying a ET1-binding ligand, the method comprising:
providing the library of claim 48,
contacting proteins from the library or encoded by the library with a target protein that comprises the protease domain of ET1, and
identifying one or more members of the library that interact with the target protein.

50. A nucleic acid comprising a sequence encoding the protein of claim 29.


SEQUENCE LISTING (217 sequences)

SEQ ID NO: 1 - protein, 422 aa, Homo sapiens
MYRPDVVRARKRVCWEPWVIGLVIFISLIVLAVCIGLTVHYVRYNQKKTYNYYSTLSFTTDKLYAEFGREASNNFTEMSQRLESMVKNAFYKSPLREEFVKSQVIKFSQQKHGVLAHMLLICRFHSTEDPETVDKIVQLVLHEKLQDAVGPPKVDPHSVKIKKINKTETDSYLNHCCGTRRSKTLGQSLRIVGGTEVEEGEWPWQASLQWDGSHRCGATLINATWLVSAAHCFTTYKNPARWTASFGVTIKPSKMKRGLRRIIVHEKYKHPSHDYDISLAELSSPVPYTNAVHRVCLPDASYEFQPGDVMFVTGFGALKNDGYSQNHLRQAQVTLIDATTCNEPQAYNDAITPRMLCAGSLEGKTDACQGDSGGPLVSSDARDIWYLAGIVSWGDECAKPNKPGVYTRVTALRDWITSKTGI

SEQ ID NO: 2 - DNA, 1352 bp, Homo sapiens
tgacttggatgtagacctcgaccttcacaggactcttcattgctggttggcaatgatgtatcggccagatgtggtgagggctaggaaaagagtttgttgggaaccctgggttatcggcctcgtcatcttcatatccctgattgtcctggcagtgtgcattggactcactgttcattatgtgagatataatcaaaagaagacctacaattactatagcacattgtcatttacaactgacaaactatatgctgagtttggcagagaggcttctaacaattttacagaaatgagccagagacttgaatcaatggtgaaaaatgcattttataaatctccattaagggaagaatttgtcaagtctcaggttatcaagttcagtcaacagaagcatggagtgttggctcatatgctgttgatttgtagatttcactctactgaggatcctgaaactgtagataaaattgttcaacttgttttacatgaaaagctgcaagatgctgtaggaccccctaaagtagatcctcactcagttaaaattaaaaaaatcaacaagacagaaacagacagctatctaaaccattgctgcggaacacgaagaagtaaaactctaggtcagagtctcaggatcgttggtgggacagaagtagaagagggtgaatggccctggcaggctagcctgcagtgggatgggagtcatcgctgtggagcaaccttaattaatgccacatggcttgtgagtgctgctcactgttttacaacatataagaaccctgccagatggactgcttcctttggagtaacaataaaaccttcgaaaatgaaacggggtctccggagaataattgtccatgaaaaatacaaacacccatcacatgactatgatatttctcttgcagagctttctagccctgttccctacacaaatgcagtacatagagtttgtctccctgatgcatcctatgagtttcaaccaggtgatgtgatgtttgtgacaggatttggagcactgaaaaatgatggttacagtcaaaatcatcttcgacaagcacaggtgactctcatagacgctacaacttgcaatgaacctcaagcttacaatgacgccataactcctagaatgttatgtgctggctccttagaaggaaaaacagatgcatgccagggtgactctggaggaccactggttagttcagatgctagagatatctggtaccttgctggaatagtgagctggggagatgaatgtgcgaaacccaacaagcctggtgtttatactagagttacggccttgcgggactggattacttcaaaaactggtatctaagagagaaaagcctcatggaacagataac

SEQ ID NO: 3 - protein, 233 aa, Homo sapiens
RIVGGTEVEEGEWPWQASLQWDGSHRCGATLINATWLVSAAHCFTTYKNPARWTASFGVTIKPSKMKRGLRRIIVHEKYKHPSHDYDISLAELSSPVPYTNAVHRVCLPDASYEFQPGDVMFVTGFGALKNDGYSQNHLRQAQVTLIDATTCNEPQAYNDAITPRMLCAGSLEGKTDACQGDSGGPLVSSDARDIWYLAGIVSWGDECAKPNKPGVYTRVTALRDWITSKTGI

SEQ ID NO: 4 - DNA, 702 bp, Homo sapiens
aggatcgttggtgggacagaagtagaagagggtgaatggccctggcaggctagcctgcagtgggatgggagtcatcgctgtggagcaaccttaattaatgccacatggcttgtgagtgctgctcactgttttacaacatataagaaccctgccagatggactgcttcctttggagtaacaataaaaccttcgaaaatgaaacggggtctccggagaataattgtccatgaaaaatacaaacacccatcacatgactatgatatttctcttgcagagctttctagccctgttccctacacaaatgcagtacatagagtttgtctccctgatgcatcctatgagtttcaaccaggtgatgtgatgtttgtgacaggatttggagcactgaaaaatgatggttacagtcaaaatcatcttcgacaagcacaggtgactctcatagacgctacaacttgcaatgaacctcaagcttacaatgacgccataactcctagaatgttatgtgctggctccttagaaggaaaaacagatgcatgccagggtgactctggaggaccactggttagttcagatgctagagatatctggtaccttgctggaatagtgagctggggagatgaatgtgcgaaacccaacaagcctggtgtttatactagagttacggccttgcgggactggattacttcaaaaactggtatctaa

SEQ ID NO: 5 - protein, 64 aa, Artificial Sequence
  Synthetically generated peptide
XXXXCXXXXXXXXXCXXXXXXXXXXXXXXXXXXCXXXXXXXCXXXXXXXXXXXXXXCXXXCXXX

SEQ ID NO: 6 - protein, 68 aa, Artificial Sequence
  Synthetically generated peptide
MHSFCAFKADXGXCXXXXXRFFFNIFTRQCEEFXYGGCXXNQNRFESLEECKKMCTRDSASSASGDFD

SEQ ID NO: 7 - protein, 51 aa, Artificial Sequence
  Synthetically generated peptide
CAFKADXGXCXAXXXRFFFNIFTRQCEEFXYGGCXXNQNRFESLEECKKMC

SEQ ID NO: 8 - protein, 68 aa, Artificial Sequence
  Synthetically generated peptide
MHSFCAFKADXGXCXAXXXRFFFNIFTRQCEEFXYGGCXXNQNRFESLEECKKMCTRDSASSASGDFD

SEQ ID NO: 9 - protein, 14 aa, Artificial Sequence
  Synthetically generated peptide
RRKCISRDIPCVTH

SEQ ID NO: 10 - protein, 14 aa, Artificial Sequence
  Synthetically generated peptide
RRYCISRDIPCVTH

SEQ ID NO: 11 - protein, 14 aa, Artificial Sequence
  Synthetically generated peptide
RVRCISRDIPCVTH

SEQ ID NO: 12 - protein, 14 aa, Artificial Sequence
  Synthetically generated peptide
RRFCISRDIPCVTH

SEQ ID NO: 13 - protein, 14 aa, Artificial Sequence
  Synthetically generated peptide
RVKCISRDIPCVTH

SEQ ID NO: 14 - protein, 14 aa, Artificial Sequence
  Synthetically generated peptide
KMRCISRDIPCTVK

SEQ ID NO: 15 - protein, 14 aa, Artificial Sequence
  Synthetically generated peptide
KMRCLSRDIPCSIH

SEQ ID NO: 16 - protein, 14 aa, Artificial Sequence
  Synthetically generated peptide
KMRCLSRDIPCVNF

SEQ ID NO: 17 - protein, 14 aa, Artificial Sequence
  Synthetically generated peptide
KMRCISRDIPCTVF

SEQ ID NO: 18 - protein, 14 aa, Artificial Sequence
  Synthetically generated peptide
KMRCISRDIPCTTR

SEQ ID NO: 19 - protein, 14 aa, Artificial Sequence
  Synthetically generated peptide
KMRCISRDIPCSHY

SEQ ID NO: 20 - protein, 14 aa, Artificial Sequence
  Synthetically generated peptide
RYPCKGFYPDCGYP

SEQ ID NO: 21 - protein, 14 aa, Artificial Sequence
  Synthetically generated peptide
GWRCKGYYPDCGYP

SEQ ID NO: 22 - protein, 14 aa, Artificial Sequence
  Synthetically generated peptide
SWRCKGYYPDCGYP

SEQ ID NO: 23 - protein, 14 aa, Artificial Sequence
  Synthetically generated peptide
TWVCKGYYPDCGYP

SEQ ID NO: 24 - protein, 14 aa, Artificial Sequence
  Synthetically generated peptide
GWRCKGYYPDCGYP

SEQ ID NO: 25 - protein, 14 aa, Artificial Sequence
  Synthetically generated peptide
GWKCKGYYPDCGYP

SEQ ID NO: 26 - protein, 14 aa, Artificial Sequence
  Synthetically generated peptide
GWRCKGYYPDCGYP

SEQ ID NO: 27 - protein, 14 aa, Artificial Sequence
  Synthetically generated peptide
KHICRGFYPDCVWQ

SEQ ID NO: 28 - protein, 14 aa, Artificial Sequence
  Synthetically generated peptide
KHICRGYYPDCVWQ

SEQ ID NO: 29 - protein, 14 aa, Artificial Sequence
  Synthetically generated peptide
KHICRGYYPDCIWQ

SEQ ID NO: 30 - protein, 14 aa, Artificial Sequence
  Synthetically generated peptide
KHICRGFYPDCVWQ

SEQ ID NO: 31 - protein, 14 aa, Artificial Sequence
  Synthetically generated peptide
KHICRGYYPDCEWQ

SEQ ID NO: 32 - protein, 16 aa, Artificial Sequence
  Synthetically generated peptide
QMRRKCISRDIPCVTH

SEQ ID NO: 33 - protein, 16 aa, Artificial Sequence
  Synthetically generated peptide
QVRRYCISRDIPCVTH

SEQ ID NO: 34 - protein, 16 aa, Artificial Sequence
  Synthetically generated peptide
RSRVRCISRDIPCVTH

SEQ ID NO: 35 - protein, 16 aa, Artificial Sequence
  Synthetically generated peptide
SGRRFCISRDIPCVTH

SEQ ID NO: 36 - protein, 16 aa, Artificial Sequence
  Synthetically generated peptide
MARVKCISRDIPCVTH

SEQ ID NO: 37 - protein, 16 aa, Artificial Sequence
  Synthetically generated peptide
AGKMRCISRDIPCTVK

SEQ ID NO: 38 - protein, 16 aa, Artificial Sequence
  Synthetically generated peptide
AGKMRCLSRDIPCSIH

SEQ ID NO: 39 - protein, 16 aa, Artificial Sequence
  Synthetically generated peptide
AGKMRCLSRDIPCVNF

SEQ ID NO: 40 - protein, 16 aa, Artificial Sequence
  Synthetically generated peptide
AGKMRCISRDIPCTVF

SEQ ID NO: 41 - protein, 16 aa, Artificial Sequence
  Synthetically generated peptide
AGKMRCISRDIPCTTR

SEQ ID NO: 42 - protein, 16 aa, Artificial Sequence
  Synthetically generated peptide
AGKMRCISRDIPCSHY

SEQ ID NO: 43 - protein, 16 aa, Artificial Sequence
  Synthetically generated peptide
GWRYPCKGFYPDCGYP

SEQ ID NO: 44 - protein, 16 aa, Artificial Sequence
  Synthetically generated peptide
NTGWRCKGYYPDCGYP

SEQ ID NO: 45 - protein, 16 aa, Artificial Sequence
  Synthetically generated peptide
RASWRCKGYYPDCGYP

SEQ ID NO: 46 - protein, 16 aa, Artificial Sequence
  Synthetically generated peptide
RETWVCKGYYPDCGYP

SEQ ID NO: 47 - protein, 16 aa, Artificial Sequence
  Synthetically generated peptide
RAGWRCKGYYPDCGYP

SEQ ID NO: 48 - protein, 16 aa, Artificial Sequence
  Synthetically generated peptide
QLGWKCKGYYPDCGYP

SEQ ID NO: 49 - protein, 16 aa, Artificial Sequence
  Synthetically generated peptide
SSGWRCKGYYPDCGYP

SEQ ID NO: 50 - protein, 16 aa, Artificial Sequence
  Synthetically generated peptide
AGKHICRGFYPDCVWQ

SEQ ID NO: 51 - protein, 16 aa, Artificial Sequence
  Synthetically generated peptide
AGKHICRGYYPDCVWQ

SEQ ID NO: 52 - protein, 16 aa, Artificial Sequence
  Synthetically generated peptide
AGKHICRGYYPDCIWQ

SEQ ID NO: 53 - protein, 16 aa, Artificial Sequence
  Synthetically generated peptide
AGKHICRGFYPDCVWQ

SEQ ID NO: 54 - protein, 16 aa, Artificial Sequence
  Synthetically generated peptide
AGKHICRGYYPDCEWQ

SEQ ID NO: 55 - protein, 6 aa, Artificial Sequence
  Synthetically generated peptide
KGFAPD

SEQ ID NO: 56 - protein, 6 aa, Artificial Sequence
  Synthetically generated peptide
KGFWPD

SEQ ID NO: 57 - protein, 4 aa, Artificial Sequence
  Synthetically generated peptide
KGPD

SEQ ID NO: 58 - protein, 6 aa, Artificial Sequence
  Synthetically generated peptide
KGLVPE

SEQ ID NO: 59 - protein, 6 aa, Artificial Sequence
  Synthetically generated peptide
KGYAPD

SEQ ID NO: 60 - protein, 6 aa, Artificial Sequence
  Synthetically generated peptide
KGYYPD

SEQ ID NO: 61 - protein, 6 aa, Artificial Sequence
  Synthetically generated peptide
KGYWPD

SEQ ID NO: 62 - protein, 6 aa, Artificial Sequence
  Synthetically generated peptide
KGYFPD

SEQ ID NO: 63 - protein, 6 aa, Artificial Sequence
  Synthetically generated peptide
KGYEPD

SEQ ID NO: 64 - protein, 6 aa, Artificial Sequence
  Synthetically generated peptide
KDYPPD

SEQ ID NO: 65 - protein, 4 aa, Artificial Sequence
  Synthetically generated peptide
KGPD

SEQ ID NO: 66 - protein, 6 aa, Artificial Sequence
  Synthetically generated peptide
RGFYPD

SEQ ID NO: 67 - protein, 6 aa, Artificial Sequence
  Synthetically generated peptide
RGFWPD

SEQ ID NO: 68 - protein, 6 aa, Artificial Sequence
  Synthetically generated peptide
RGYAPD

SEQ ID NO: 69 - protein, 6 aa, Artificial Sequence
  Synthetically generated peptide
LSRDTP

SEQ ID NO: 70 - protein, 6 aa, Artificial Sequence
  Synthetically generated peptide
LSRDLP

SEQ ID NO: 71 - protein, 6 aa, Artificial Sequence
  Synthetically generated peptide
ESRDLP

SEQ ID NO: 72 - protein, 6 aa, Artificial Sequence
  Synthetically generated peptide
ESRDIP

SEQ ID NO: 73 - protein, 5 aa, Artificial Sequence
  Synthetically generated peptide
TRDLP

SEQ ID NO: 74 - protein, 57 aa, Artificial Sequence
  Synthetically generated peptide
SFCAFKADRGPCRADFHRFFFNIFTRQCEEFHYGGCGGNQNRFESLEECKKMCTRDS

SEQ ID NO: 75 - protein, 57 aa, Artificial Sequence
  Synthetically generated peptide
SFCAFKADKGFCRAMDIRFFFNIFTRQCEEFIYGGCGGNQNRFESLEECKKMCTRDS

SEQ ID NO: 76 - protein, 57 aa, Artificial Sequence
  Synthetically generated peptide
SFCAFKADQGPCRAAISRFFFNIFTRQCEEFVYGGCEGNQNRFESLEECKKMCTRDS

SEQ ID NO: 77 - protein, 57 aa, Artificial Sequence
  Synthetically generated peptide
SFCAFKADKGECRASVQRFFFNIFTRQCEEFNYGGCGGNQNRFESLEECKKMCTRDS

SEQ ID NO: 78 - protein, 57 aa, Artificial Sequence
  Synthetically generated peptide
SFCAFKADPGPCRAMFNRFFFNIFTRQCEEFNYGGCSGNQNRFESLEECKKMCTRDS

SEQ ID NO: 79 - protein, 57 aa, Artificial Sequence
  Synthetically generated peptide
SFCAFKADKGTCRGDFPRFFFNIFTRQCEEFHYGGCGGNQNRFESLEECKKMCTRDS

SEQ ID NO: 80 - protein, 57 aa, Artificial Sequence
  Synthetically generated peptide
SFCAFKADQGPCRASVHRFFFNIFTRQCEEFFYGGCLGNQNRFESLEECKKMCTRDS

SEQ ID NO: 81 - protein, 57 aa, Artificial Sequence
  Synthetically generated peptide
SFCAFKADPGQCRAYYRRFFFNIFTRQCEEFVYGGCMGNQNRFESLEECKKMCTRDS

SEQ ID NO: 82 - protein, 57 aa, Artificial Sequence
  Synthetically generated peptide
SFCAFKADRGPCRAYFDRFFFNIFTRQCEEFIYGGCMGNQNRFESLEECKKMCTRDS

SEQ ID NO: 83 - protein, 57 aa, Artificial Sequence
  Synthetically generated peptide
SFCAFKADTGPCRADIKRFFFNIFTRQCEEFRYGGCMGNQNRFESLEECKKMCTRDS

SEQ ID NO: 84 - protein, 57 aa, Artificial Sequence
  Synthetically generated peptide
SFCAFKADPGPCRAIMTRFFFNIFTRQCEEFRYGGCLGNQNRFESLEECKKMCTRDS

SEQ ID NO: 85 - protein, 57 aa, Artificial Sequence
  Synthetically generated peptide
SFCAFKADTGTCRAAMVRFFFNIFTRQCEEFTYGGCEGNQNRFESLEECKKMCTRDS

SEQ ID NO: 86 - protein, 16 aa, Artificial Sequence
  Synthetically generated peptide
AGFQKCKGPDCYVPGT

SEQ ID NO: 87 - protein, 17 aa, Artificial Sequence
  Synthetically generated peptide
AGMMMCKGLVPECKGGT

SEQ ID NO: 88 - protein, 18 aa, Artificial Sequence
  Synthetically generated peptide
AGTAHCFTKDFPCIIFGT

SEQ ID NO: 89 - protein, 20 aa, Artificial Sequence
  Synthetically generated peptide
GSHSVCTRDLPISYCVPNAP

SEQ ID NO: 90 - protein, 18 aa, Artificial Sequence
  Synthetically generated peptide
AGHWNCKGFAPDCEFIGT

SEQ ID NO: 91 - protein, 18 aa, Artificial Sequence
  Synthetically generated peptide
AGHWQCKGFWPDCIPSAT

SEQ ID NO: 92 - protein, 22 aa, Artificial Sequence
  Synthetically generated peptide
GDRSPCGRWGKTDTKMCQDWDP

SEQ ID NO: 93 - protein, 18 aa, Artificial Sequence
  Synthetically generated peptide
AGIKHCLSRDTPCITFGT

SEQ ID NO: 94 - protein, 18 aa, Artificial Sequence
  Synthetically generated peptide
AGVQHCESRDLPCLIKGT

SEQ ID NO: 95 - protein, 23 aa, Artificial Sequence
  Synthetically generated peptide
GDRGDCEVKMYPWPDKCKHRDPT

SEQ ID NO: 96 - protein, 18 aa, Artificial Sequence
  Synthetically generated peptide
AGAGKCKGFWPDCYHQGT

SEQ ID NO: 97 - protein, 18 aa, Artificial Sequence
  Synthetically generated peptide
AGHWQCKGYAPDCEPWGT

SEQ ID NO: 98 - protein, 19 aa, Artificial Sequence
  Synthetically generated peptide
AGAHTCESRDIPCTVKGTY

SEQ ID NO: 99 - protein, 18 aa, Artificial Sequence
  Synthetically generated peptide
AGKWHCKGYAPDCQMWGT

SEQ ID NO: 100 - protein, 18 aa, Artificial Sequence
  Synthetically generated peptide
AGKHICKGYYPDCGYPGT

SEQ ID NO: 101 - protein, 18 aa, Artificial Sequence
  Synthetically generated peptide
AGEYRCKGYWPDCASFGT

SEQ ID NO: 102 - protein, 19 aa, Artificial Sequence
  Synthetically generated peptide
GSSWYCDKAHPARCWNPAP

SEQ ID NO: 103 - protein, 18 aa, Artificial Sequence
  Synthetically generated peptide
AGMWSCKGYFPDCSNMGT

SEQ ID NO: 104 - protein, 18 aa, Artificial Sequence
  Synthetically generated peptide
AGNYMCKGYWPDCKMTGT

SEQ ID NO: 105 - protein, 18 aa, Artificial Sequence
  Synthetically generated peptide
AGEFPCRGFYPDCGYMGT

SEQ ID NO: 106 - protein, 18 aa, Artificial Sequence
  Synthetically generated peptide
AGHFMCRGYAPDCKPWGT

SEQ ID NO: 107 - protein, 19 aa, Artificial Sequence
  Synthetically generated peptide
AGKMRCLSRDLPCVTHGTY

SEQ ID NO: 108 - protein, 18 aa, Artificial Sequence
  Synthetically generated peptide
AGWWPCKGYEPDCPTNGT

SEQ ID NO: 109 - protein, 18 aa, Artificial Sequence
  Synthetically generated peptide
AGAWLCKGYPPDCAQQGT

SEQ ID NO: 110 - protein, 18 aa, Artificial Sequence
  Synthetically generated peptide
AGIGMCKGYPPDCIGRGT

SEQ ID NO: 111 - protein, 16 aa, Artificial Sequence
  Synthetically generated peptide
AGNWYCKGPDCMHKGT

SEQ ID NO: 112 - protein, 18 aa, Artificial Sequence
  Synthetically generated peptide
AGWPTCRGFWPDCGMMGT

SEQ ID NO: 113 - protein, 24 aa, Artificial Sequence
  Synthetically generated peptide
AQGDNIGVWLWAPYSKGFAWQLGG

SEQ ID NO: 114 - protein, 24 aa, Artificial Sequence
  Synthetically generated peptide
AQNREHSSKFGTVRYSTLGPPPGG

SEQ ID NO: 115 - protein, 24 aa, Artificial Sequence
  Synthetically generated peptide
AQGMQDEGGAIRHKGAWYWMMAGG

SEQ ID NO: 116 - protein, 16 aa, Artificial Sequence
  Synthetically generated peptide
GSQHICHPGGCEKPAP

SEQ ID NO: 117 - protein, 18 aa, Artificial Sequence
  Synthetically generated peptide
AGLRKCGFWGFPCKGMGT

SEQ ID NO: 118 - protein, 22 aa, Artificial Sequence
  Synthetically generated peptide
GDYLQCRWNAWENRTLCTWRDP

SEQ ID NO: 119 - protein, 15 aa, Artificial Sequence
  Synthetically generated peptide
GSNGHCDNHCQMNAP

SEQ ID NO: 120 - protein, 18 aa, Artificial Sequence
  Synthetically generated peptide
AGGFKCISEEEDCKLMGT

SEQ ID NO: 121 - protein, 18 aa, Artificial Sequence
  Synthetically generated peptide
AGPDPCRMQGPWCTPMGT

SEQ ID NO: 122 - protein, 18 aa, Artificial Sequence
  Synthetically generated peptide
AGTEFCWLHKGICKTWGT

SEQ ID NO: 123 - protein, 18 aa, Artificial Sequence
  Synthetically generated peptide
AGTMSCDGSMVPCYTPGT

SEQ ID NO: 124 - protein, 24 aa, Artificial Sequence
  Synthetically generated peptide
AQPHWVPNQPVRDRWQSFPKWLGG

SEQ ID NO: 125 - protein, 22 aa, Artificial Sequence
  Synthetically generated peptide
GDDNECEPDADLSEYECVHRDP

SEQ ID NO: 126 - protein, 22 aa, Artificial Sequence
  Synthetically generated peptide
GDNLFCGHSKYAQDHRCRLYDP

SEQ ID NO: 127 - protein, 22 aa, Artificial Sequence
  Synthetically generated peptide
GDSPHCGSHVTVNEKSCMFYDP

SEQ ID NO: 128 - protein, 16 aa, Artificial Sequence
  Synthetically generated peptide
GSNHICPSMGCKFSAP

SEQ ID NO: 129 - protein, 16 aa, Artificial Sequence
  Synthetically generated peptide
GSSFFCVGPECWTSAP

SEQ ID NO: 130 - protein, 16 aa, Artificial Sequence
  Synthetically generated peptide
GSSMFCDAYYCTDHAP

SEQ ID NO: 131 - protein, 16 aa, Artificial Sequence
  Synthetically generated peptide
GSWDSCNELRCIWDAP

SEQ ID NO: 132 - protein, 16 aa, Artificial Sequence
  Synthetically generated peptide
GSVGLCYQNFCKKIAP

SEQ ID NO: 133 - protein, 19 aa, Artificial Sequence
  Synthetically generated peptide
AGHGECMVASHMCIKHGTY

SEQ ID NO: 134 - protein, 14 aa, Artificial Sequence
  Exemplary motif
XXXCXXXDXPCXXX

SEQ ID NO: 135 - protein, 14 aa, Artificial Sequence
  Exemplary motif
XXXCXGXYPDCXXX

SEQ ID NO: 136 - protein, 21 aa, Artificial Sequence
  Library Isolate
AGQMRRKCISRDIPCVTHGTY

SEQ ID NO: 137 - protein, 20 aa, Artificial Sequence
  Library Isolate
AGQVRRYCISRDIPCVTHGT

SEQ ID NO: 138 - protein, 21 aa, Artificial Sequence
  Library Isolate
AGRSRVRCISRDIPCVTHGTY

SEQ ID NO: 139 - protein, 20 aa, Artificial Sequence
  Library Isolate
AGSGRRFCISRDIPCVTHGT

SEQ ID NO: 140 - protein, 21 aa, Artificial Sequence
  Library Isolate
AGMARVKCISRDIPCVTHGTY

SEQ ID NO: 141 - protein, 21 aa, Artificial Sequence
  Library Isolate
AGKMRCISRDIPCTVKSGGTY

SEQ ID NO: 142 - protein, 21 aa, Artificial Sequence
  Library Isolate
AGKMRCLSRDIPCSIHQKGTY

SEQ ID NO: 143 - protein, 20 aa, Artificial Sequence
  Library Isolate
AGKMRCLSRDIPCVNFRLGT

SEQ ID NO: 144 - protein, 20 aa, Artificial Sequence
  Library Isolate
AGKMRCISRDIPCTVFQEGT

SEQ ID NO: 145 - protein, 21 aa, Artificial Sequence
  Library Isolate
AGKMRCISRDIPCTTRVAGTY

SEQ ID NO: 146 - protein, 20 aa, Artificial Sequence
  Library Isolate
AGKMRCISRDIPCSHYQIGT

SEQ ID NO: 147 - protein, 20 aa, Artificial Sequence
  Library Isolate
AGGWRYPCKGFYPDCGYPGT

SEQ ID NO: 148 - protein, 20 aa, Artificial Sequence
  Library Isolate
AGNTGWRCKGYYPDCGYPGT

SEQ ID NO: 149 - protein, 20 aa, Artificial Sequence
  Library Isolate
AGRASWRCKGYYPDCGYPGT

SEQ ID NO: 150 - protein, 20 aa, Artificial Sequence
  Library Isolate
AGRETWVCKGYYPDCGYPGT

SEQ ID NO: 151 - protein, 20 aa, Artificial Sequence
  Library Isolate
AGRAGWRCKGYYPDCGYPGT

SEQ ID NO: 152 - protein, 20 aa, Artificial Sequence
  Library Isolate
AGQLGWKCKGYYPDCGYPGT

SEQ ID NO: 153 - protein, 20 aa, Artificial Sequence
  Library Isolate
AGSSGWRCKGYYPDCGYPGT

SEQ ID NO: 154 - protein, 20 aa, Artificial Sequence
  Library Isolate
AGKHICRGFYPDCVWQTWGT

SEQ ID NO: 155 - protein, 20 aa, Artificial Sequence
  Library Isolate
AGKHICRGYYPDCVWQTWGT

SEQ ID NO: 156 - protein, 20 aa, Artificial Sequence
  Library Isolate
AGKHICRGYYPDCVWQTFGT

SEQ ID NO: 157 - protein, 20 aa, Artificial Sequence
  Library Isolate
AGKHICRGYYPDCIWQFAGT

SEQ ID NO: 158 - protein, 20 aa, Artificial Sequence
  Library Isolate
AGKHICRGFYPDCVWQTFGT

SEQ ID NO: 159 - protein, 20 aa, Artificial Sequence
  Library Isolate
AGKHICRGYYPDCEWQIFGT

SEQ ID NO: 160 - protein, 20 aa, Artificial Sequence
  Synthetically generated peptide
AGQVRRYCISRDIPCVTHGT

SEQ ID NO: 161 - protein, 21 aa, Artificial Sequence
  Synthetically generated peptide
AGQMRRKCISRDIPCVTHGTY

SEQ ID NO: 162 - protein, 21 aa, Artificial Sequence
  Synthetically generated peptide
AGRSRVRCISRDIPCVTHGTY

SEQ ID NO: 163 - protein, 20 aa, Artificial Sequence
  Synthetically generated peptide
AGSGRRFCISRDIPCVTHGT

SEQ ID NO: 164 - protein, 21 aa, Artificial Sequence
  Synthetically generated peptide
AGMARVKCISRDIPCVTHGTY

SEQ ID NO: 165 - protein, 21 aa, Artificial Sequence
  Synthetically generated peptide
AGKMRCISRDIPCTVKSGGTY

SEQ ID NO: 166 - protein, 21 aa, Artificial Sequence
  Synthetically generated peptide
AGKMRCLSRDIPCSIHQKGTY

SEQ ID NO: 167 - protein, 20 aa, Artificial Sequence
  Synthetically generated peptide
AGKMRCLSRDIPCVNFRLGT

SEQ ID NO: 168 - protein, 20 aa, Artificial Sequence
  Synthetically generated peptide
AGKMRCISRDIPCTVFQEGT

SEQ ID NO: 169 - protein, 21 aa, Artificial Sequence
  Synthetically generated peptide
AGKMRCISRDIPCTTRVAGTY

SEQ ID NO: 170 - protein, 20 aa, Artificial Sequence
  Synthetically generated peptide
AGKMRCISRDIPCSHYQIGT

SEQ ID NO: 171 - protein, 20 aa, Artificial Sequence
  Synthetically generated peptide
AGGWRYPCKGFYPDCGYPGT

SEQ ID NO: 172 - protein, 20 aa, Artificial Sequence
  Synthetically generated peptide
AGNTGWRCKGYYPDCGYPGT

SEQ ID NO: 173 - protein, 20 aa, Artificial Sequence
  Synthetically generated peptide
AGRASWRCKGYYPDCGYPGT

SEQ ID NO: 174 - protein, 20 aa, Artificial Sequence
  Synthetically generated peptide
AGRETWVCKGYYPDCGYPGT

SEQ ID NO: 175 - protein, 20 aa, Artificial Sequence
  Synthetically generated peptide
AGRAGWRCKGYYPDCGYPGT

SEQ ID NO: 176 - protein, 20 aa, Artificial Sequence
  Synthetically generated peptide
AGQLGWKCKGYYPDCGYPGT

SEQ ID NO: 177 - protein, 20 aa, Artificial Sequence
  Synthetically generated peptide
AGSSGWRCKGYYPDCGYPGT

SEQ ID NO: 178 - protein, 20 aa, Artificial Sequence
  Synthetically generated peptide
AGKHICRGFYPDCVWQTWGT

SEQ ID NO: 179 - protein, 20 aa, Artificial Sequence
  Synthetically generated peptide
AGKHICRGYYPDCVWQTWGT

SEQ ID NO: 180 - protein, 20 aa, Artificial Sequence
  Synthetically generated peptide
AGKHICRGYYPDCVWQTFGT

SEQ ID NO: 181 - protein, 20 aa, Artificial Sequence
  Synthetically generated peptide
AGKHICRGYYPDCIWQFAGT

SEQ ID NO: 182 - protein, 20 aa, Artificial Sequence
  Synthetically generated peptide
AGKHICRGFYPDCVWQTFGT

SEQ ID NO: 183 - protein, 20 aa, Artificial Sequence
  Synthetically generated peptide
AGKHICRGYYPDCEWQIFGT

SEQ ID NO: 184 - protein, 72 aa, Artificial Sequence
  Synthetically generated peptide
MAAEMHSFCAFKADRGPCRADFHRFFFNIFTRQCEEFHYGGCGGNQNRFESLEECKKMCTRDSASSASGDFD

SEQ ID NO: 185 - protein, 72 aa, Artificial Sequence
  Synthetically generated peptide
MAAEMHSFCAFKADKGFCRAMDIRFFFNIFTRQCEEFIYGGCGGNQNRFESLEECKKMCTRDSASSASGDFD

SEQ ID NO: 186 - protein, 72 aa, Artificial Sequence
  Synthetically generated peptide
MAAEMHSFCAFKADQGPCRAAISRFFFNIFTRQCEEFVYGGCEGNQNRFESLEECKKMCTRDSASSASGDFD

SEQ ID NO: 187 - protein, 72 aa, Artificial Sequence
  Synthetically generated peptide
MAAEMHSFCAFKADKGECRASVQRFFFNIFTRQCEEFNYGGCGGNQNRFESLEECKKMCTRDSASSASGDFD

SEQ ID NO: 188 - protein, 72 aa, Artificial Sequence
  Synthetically generated peptide
MAAEMHSFCAFKADPGPCRAMFNRFFFNIFTRQCEEFNYGGCSGNQNRFESLEECKKMCTRDSASSASGDFD

SEQ ID NO: 189 - protein, 72 aa, Artificial Sequence
  Synthetically generated peptide
MAAEMHSFCAFKADKGTCRGDFPRFFFNIFTRQCEEFHYGGCGGNQNRFESLEECKKMCTRDSASSASGDFD

SEQ ID NO: 190 - protein, 72 aa, Artificial Sequence
  Synthetically generated peptide
MAAEMHSFCAFKADQGPCRASVHRFFFNIFTRQCEEFFYGGCLGNQNRFESLEECKKMCTRDSASSASGDFD

SEQ ID NO: 191 - protein, 72 aa, Artificial Sequence
  Synthetically generated peptide
MAAEMHSFCAFKADPGQCRAYYRRFFFNIFTRQCEEFVYGGCMGNQNRFESLEECKKMCTRDSASSASGDFD

SEQ ID NO: 192 - protein, 72 aa, Artificial Sequence
  Synthetically generated peptide
MAAEMHSFCAFKADRGPCRAYFDRFFFNIFTRQCEEFIYGGCMGNQNRFESLEECKKMCTRDSASSASGDFD

SEQ ID NO: 193 - protein, 72 aa, Artificial Sequence
  Synthetically generated peptide
MAAEMHSFCAFKADTGPCRADIKRFFFNIFTRQCEEFRYGGCMGNQNRFESLEECKKMCTRDSASSASGDFD

SEQ ID NO: 194 - protein, 72 aa, Artificial Sequence
  Synthetically generated peptide
MAAEMHSFCAFKADPGPCRAIMTRFFFNIFTRQCEEFRYGGCLGNQNRFESLEECKKMCTRDSASSASGDFD

SEQ ID NO: 195 - protein, 72 aa, Artificial Sequence
  Synthetically generated peptide
MAAEMHSFCAFKADTGTCRAAMVRFFFNIFTRQCEEFTYGGCEGNQNRFESLEECKKMCTRDSASSASGDFD

SEQ ID NO: 196 - protein, 18 aa, Artificial Sequence
  Synthetically generated peptide
AGKMRCLSRDLPCVTHGT

SEQ ID NO: 197 - protein, 18 aa, Artificial Sequence
  Synthetically generated peptide
AGKHICKGYYPDCGYPGT

SEQ ID NO: 198 - protein, 18 aa, Artificial Sequence
  Synthetically generated peptide
AGHWQCKGYAPDCEPWGT

SEQ ID NO: 199 - protein, 18 aa, Artificial Sequence
  Synthetically generated peptide
GDRRKCISKDTPCTVHDP

SEQ ID NO: 200 - DNA, 91 bp, Artificial Sequence
  Synthetically generated oigonucleotide
tggctgcggaaacagaggctggtnnknnknnknnknnktgtmttwccarggatmytccctgtgttacacatggtactgaacctactgaaag

SEQ ID NO: 201 - protein, 34 aa, Artificial Sequence
  Synthetically generated peptide
MAAETEAGXXXXXCXXXDXPCVTHGTEPTESSAD

SEQ ID NO: 202 - DNA, 91 bp, Artificial Sequence
  Synthetically generated oliginucleotide
tggctgcggaaacagaggctggtaagatgcgctgtmttwccarggatmytccctgtnnknnknnknnknnkggtactgaacctactgaaag

SEQ ID NO: 203 - protein, 26 aa, Artificial Sequence
  Synthetically generated peptide
KMRCXXXDXPCXXXXXGTEPTESSAD

SEQ ID NO: 204 - DNA, 91 bp, Artificial Sequence
  Synthetically generated oligonucleotide
tggctgcggaaacagaggctggtnnknnknnknnknnktgtargggttwttatcctgattgtggttatcccggtactgaacctactgaaag

SEQ ID NO: 205 - protein, 34 aa, Artificial Sequence
  Synthetically generated peptide
MAAETEAGXXXXXCXGXYPDCGYPGTEPTESSAD

SEQ ID NO: 206 - DNA, 91 bp, Artificial Sequence
  Synthetically generated oligonucleotide
tggctgcggaaacagaggctggtaagcatatttgtargggttwttatcctgattgtnnknnknnknnknnkggtactgaacctactgaaag

SEQ ID NO: 207 - protein, 26 aa, Artificial Sequence
  Synthetically generated peptide
KHICXGXYPDCXXXXXGTEPTESSAD

SEQ ID NO: 208 - protein, 18 aa, Artificial Sequence
  Synthetically generated peptide
AGAHTCESRDIPCTVKGT

SEQ ID NO: 209 - protein, 8 aa, Artificial Sequence
  Synthetically generated peptide
CXXRDXPC

SEQ ID NO: 210 - protein, 14 aa, Artificial Sequence
  Synthetically generated peptide
XXXCXSRDLPCXXX

SEQ ID NO: 211 - protein, 7 aa, Artificial Sequence
  Synthetically generated peptide
CKGXPDC

SEQ ID NO: 212 - protein, 14 aa, Artificial Sequence
  Synthetically generated peptide
XXXCXXXXXXCXXX

SEQ ID NO: 213 - protein, 8 aa, Artificial Sequence
  Synthetically generated peptide
CXXXXXXC

SEQ ID NO: 214 - protein, 8 aa, Artificial Sequence
  Synthetically generated peptide
CXXXXXXC

SEQ ID NO: 215 - protein, 11 aa, Artificial Sequence
  Synthetically generated peptide
XXXCXXXXXXC

SEQ ID NO: 216 - protein, 11 aa, Artificial Sequence
  Synthetically generated peptide
CXXXXXXCXXX

SEQ ID NO: 217 - protein, 13 aa, Artificial Sequence
  Synthetically generated peptide
CXXXXXXCXXXXX